(19) United States
(12) Patent Application Publication
(10) Pub. No.: US 2009/0311267 A1
(43) Pub. Date: Dec. 17, 2009

(54) Inhibition of VWF - GPIb/V/IX interaction and platelet-collagen interaction for prevention and treatment of cerebral attacks

(75) Inventors: Guido Stoll, Rimpar (DE); Christoph Kleinschnitz, Estenfeld (DE); Bernhard Nieswandt, Eibelstadt (DE); Hans Deckmyn, Linden (BE); Simon De Meyer, Heule (BE)

Correspondence Address: TRASKBRITT, P.C., P.O. BOX 2550, SALT LAKE CITY, UT, 84110, US

(73) Assignees: University of Wurzburg, Wurzburg (DE); Katholieke Universiteit Leuven, K.U. Leuven R&D, Leuven (BE)

(21) Appl. No.: 12/383,315

(22) Filed: Mar. 23, 2009

Related U.S. Application Data
(63) Continuation-in-part of application No. 11/963,687, filed on Dec. 21, 2007.
Application No. 11/963,687 is a division of application No. 10/049,868, filed on Jun. 4, 2002, now Pat. No. 7,332,162.
(60) Provisional application No. 61/124,293, filed on Apr. 15, 2008.

(30) Foreign Application Priority Data
Aug. 10, 1999 (GB) 9918788.2
Feb. 2, 2000 (EP) 00102032.0

(51) Int. Cl. A61K 39/395

(52) U.S. Cl. 424/145.1; 424/130.1

(57) ABSTRACT

The invention relates generally to an anti-thrombotic treatment of occlusive syndromes in the cerebral vascular system causing cerebral infarct due to stroke or ischemic stroke, which is a major cause of death and permanent disability in industrialized countries. More particularly, the invention relates to a system and method of preventing and treating such occlusive syndromes in the cerebral vascular by inhibiting initial adhesion/attachment of platelets to the endothelium by preventing or inhibiting binding of von Willebrand factor to platelet glycoprotein Ib by administration of a subject in such need anti-glycoprotein Ib monovalent antibodies and/or anti-vWF monovalent antibodies, rather than by blocking the common pathway of platelet aggregation by blockade of platelet aggregation with anti-glycoprotein IIb/IIIa.

### CROSS-REFERENCE TO RELATED APPLICATIONS

This application claims the benefit under 35 U.S.C. § 119(e) of U.S. Provisional Application Ser. No. 61/124,293, filed Apr. 15, 2008, and is a continuation-in-part of U.S. application Ser. No. 11/963,687, filed Dec. 21, 2007, pending, which is a divisional of U.S. application Ser. No. 10/049,868, filed Jun. 4, 2002, which is now U.S. Pat. No. 7,332,162. U.S. application Ser. No. 11/963,687 is also a National Stage Entry of PCT/EP2000/007874 filed Aug. 8, 2000, which claims priority to European Patent Application No. 00102032.0, filed Feb. 2, 2000 and United Kingdom Application No 9918788.2, filed Aug. 10, 1999. The entirety of each of the previously referenced patent applications and patents referenced is hereby incorporated herein by reference.

### TECHNICAL FIELD

The invention relates generally to an anti-thrombotic treatment of occlusive syndromes in the cerebral vascular system causing cerebral infarcts due to stroke or ischemic stroke, which is a major cause of death and permanent disability in industrialized countries.

More particularly, the invention relates to a system and method of preventing or treating such occlusive syndromes in the cerebral vasculature by inhibiting initial adhesion/attachment of platelets to the endothelium by preventing or inhibiting binding of von Willebrand factor or other ligands to platelet glycoprotein Ib by administration to a subject in such need of anti-glycoprotein Ib monovalent antibodies and/or anti-VWF antibodies, rather than by blocking the common pathway of platelet aggregation by blockade of platelet aggregation with anti-glycoprotein IIb/IIIa antibodies.

Cell lines, ligands and antibody fragments for use in a pharmaceutical composition for preventing and treating a hemostasis disorder, in particular, such hemostasis disorder, which is an occlusive syndrome in the cerebral vascular system causing transient cerebral infarct due to stroke or ischemic stroke, are also part of the invention.

### BACKGROUND

Several documents are cited throughout the text of this specification. Each of the documents herein (including any manufacturer's specifications, instructions, etc.) are hereby incorporated by reference; however, there is no admission that any document cited is indeed prior art to the invention.

Ischemic stroke is a frequent and serious disease with limited treatment options. Platelets can adhere to hypoxic cerebral endothelial cells by binding of their glycoprotein (GP) Ib receptor to von Willebrand factor. Exposure of subendothelial matrix proteins further facilitates firm attachment of platelets to the vessel wall by binding of collagen to their GPVI receptor as the GPVI receptor mediates both adhesion and signaling responses to collagen in a receptor density-dependent fashion.

Ischemic stroke is the third-leading cause of death and permanent disability in industrialized countries (C. J. Murray et al., Lancet. 1997, 349:1269-1276). Although the beneficial role of anti-coagulation and the platelet aggregation inhibitors acetylsalicylic acid, clopidogrel, and dipyridamole in stroke prevention is well established, their use in the acute phase of cerebral ischemia is a matter of debate. A moderate benefit on stroke progression and recurrence is often outweighed by a significant increase in the rate of intracerebral hemorrhage (ICH) (P. Sandercock et al., Cochrane Database Syst. Rev. 2003, 2:CD000029; E. L. De Schryver et al., Cochrane Database Syst. Rev. 2006, 2:CD001820; D. L. Bhatt et al., N. Engl. J. Med. 2006, 354:1706-1717; and G. J. del Zoppo, “The role of platelets in ischemic stroke,” Neurology 1998 (Suppl. 3) 51:S9-S14).

Recently, major progress has been made in the functional analysis of platelet activation and platelet-dependent thrombus formation. Platelets can adhere to hypoxic endothelial cells by binding of their glycoprotein (GP) Ib receptor to von Willebrand factor (vWF) on the endothelial surface (R. K. Andrews and M. C. Berndt, “Platelet physiology and thrombosis,” Thromb. Res. 2004, 114:447-453). Exposure of subendothelial matrix proteins during ischemia further facilitates firm attachment of platelets to the vessel wall by binding of collagens to their GPVI receptor (B. Savage et al., Cell. 1996, 84:289-297; and B. Kehrel, Semin. Thromb. Hemost. 1995, 21:123-129). These processes lead to activation of platelet GPIIb/IIIa and platelet aggregation (D. R. Phillips et al., Cell. 1991, 65:359-362). Although it is well established that endothelial cells undergo activation (G. J. del Zoppo and T. Mabuchi, “Cerebral microvessel responses to focal ischemia, J. Cereb. Blood Flow Metab. 2003, 23:879-894) and that microvascular integrity is disturbed during cerebral ischemia (Z. G. Zhang et al., Brain Res. 2001, 912:181-194), less is known about the signaling cascades that lead to intravascular thrombus formation in the brain.

It was recently demonstrated that coagulation factor XII plays a decisive role in thrombus formation after transient focal cerebral ischemia (C. Kleinschnitz et al., J. Exp. Med. 2006, 203:513-518).

### DISCLOSURE OF THE INVENTION

By the invention, we show that targeting platelet GPIb or GPVI receptors protects mice from ischemic brain injury in an experimental stroke model without an increase in bleeding complications. In contrast, blockade of the final common pathway of platelet aggregation with anti-GPIIb/IIIa antibodies had no positive effect on stroke outcome and dose-dependently raised the incidence of ICH and mortality.

We addressed the pathogenic role of GPIb, GPVI, and the aggregation receptor GPIIb/IIIa in experimental stroke in mice and found that the targeting by inhibiting monovalent antibodies of platelet glycoprotein Ib and glycoprotein VI receptors, which mediate initial adhesion/attachment of platelets to endothelial cells and the subendothelial matrix, protected mice from ischemic brain injury after transient occlusion of the middle cerebral artery. This is not associated with an increase in bleeding complications as revealed by magnetic resonance imaging. Delayed treatment with anti-glycoprotein Ib monovalent antibody, such as a Fab, during reperfusion was similarly effective. In contrast, blockade of platelet aggregation with anti-glycoprotein IIb/IIIa F(ab)₂ had no positive effect on stroke outcome but raised the incidence of intracerebral hemorrhages in a dose-dependent manner.

Subjects suffering of or at risk of occlusive syndrome in the cerebral vascular system, of transient cerebral attacks or of cerebral thrombosis resulting in cerebral infarction referred to as stroke, ischemic stroke or acute stroke are, according to the invention, treated by a monovalent antibody against platelet glycoprotein GPIb that inhibits or prevents the activation of GPIb-mediated pathways leading to thrombus formation in the cerebral vascular system, e.g., by a monovalent antibody against platelet glycoprotein GPIb that inhibits or prevents binding of von Willebrand factor to the human platelet glycoprotein GPIb or bivalent or monovalent antibodies against von Willebrand factor or monovalent antibodies against glycoprotein VI (GPVI) to protect a subject from ischemic brain injury in a stroke without an increase in bleeding complications by inhibiting signaling responses to collagen and inhibiting initial adhesion/attachment of platelets to endothelial cells and the subendothelial matrix of the vessel wall by binding of collagen to platelet GPVI receptor or to vWF.

In certain embodiments, subjects suffering from, or at risk of, occlusive syndrome in the cerebral vascular system or of cerebral thrombosis resulting in transient cerebral infarct such as (ischemic) stroke are treated by a monovalent anti-VWF antibody. Such monovalent antibody can, e.g., be a fab, a fab′ or a ScFv comprising both a heavy chain variable domain and/or a light chain variable domain or such monovalent antibody can be anti-vWF single domain fragments comprising only the variable domain. The anti-vWF antibody can be a bivalent such as IgG and Fab2.

A treatment of inhibiting or preventing of binding of von Willebrand factor to the human platelet glycoprotein GPIb can also be combined with inhibitors or neutralizers of alpha-2-antiplasmin (α 2-AP) or (α 2-AP) binding compounds such as human plasmin, human plasmin-forming proteins, including lys-plasminogen, antibodies or antibody fragments or derivatives to alpha-2-antiplasmin or similar substances to enhance fibrinolysis, e.g., by lowering the concentration of α2-APor lowering its activity.

The invention concerns, in particular, a monovalent anti-GPIb antibody for the treatment or prevention of transient cerebral attack leading to cerebral ischemia, of stroke, of ischemic stroke, or of acute stroke, which anti-GPIb antibody has an improved safety profile and decreased (intracerebral) hemorrhages versus a treatment of blocking the common pathway of platelet aggregation by blockade of platelet aggregation with anti-glycoprotein IIb/IIIa. A problem related to the current treatment of stroke is that (intracerebral) hemorrhages may counterbalance the benefit of therapeutic or prophylactic use of the established anti-platelet or anti-coagulant drugs in acute cerebral ischemia. There was thus a need in the art for a more favorable safety profile of a treatment of ischemic brain injury or brain infarct after thromboembolic occlusion in the cerebral vascular system.

The invention solves the problems of the related art of increase in bleeding complications by the current stroke treatments. The only approved therapy during the acute stroke phase is thrombolysis within a narrow time window of three hours (up to six hours), but the number of patients amenable to this treatment is low. Moreover, intracerebral hemorrhages may counterbalance the benefit of therapeutic or prophylactic use of the established anti-platelet or anti-coagulant drugs in acute cerebral ischemia. By the invention, it was demonstrated that targeting platelet glycoprotein Ib and glycoprotein VI receptors, which mediate initial adhesion/attachment of platelets to endothelial cells and the subendothelial matrix, protects mice from ischemic brain injury after transient occlusion of the middle cerebral artery. Importantly, this was not associated with an increase in bleeding complications as revealed by magnetic resonance imaging. Delayed treatment with anti-glycoprotein Ib Fab during reperfusion was similarly effective. In contrast, blockade of platelet aggregation with anti-glycoprotein IIb/IIIa F(ab)₂ had no positive effect on stroke outcome but raised the incidence of intracerebral hemorrhages in a dose-dependent manner.

The invention is broadly drawn to targeting initial adhesion/attachment of platelets to the endothelium rather than blocking the common pathway of platelet aggregation, which opens new avenues for acute stroke treatment with a more favorable safety profile.

In a mice model, complete blockade of GPIbα was achieved by intravenous injection of 100 μg Fab fragments of the monoclonal antibody p0p/B to mice undergoing one hour of transient middle cerebral artery occlusion. At 24 hours after transient middle cerebral artery occlusion, cerebral infarct volumes were assessed by 2,3,5-triphenyltetrazolium chloride staining. In mice treated with anti-GPIbα Fab one hour before middle cerebral artery occlusion, ischemic lesions were reduced to ≈40% compared with controls (28.5±12.7 versus 73.9±17.4 mm³, respectively; P<0.001). Application of anti-GPIbα Fab one hour after middle cerebral artery occlusion likewise reduced brain infarct volumes (24.5±7.7 mm³; P<0.001) and improved the neurological status. Similarly, depletion of GPVI significantly diminished the infarct volume but to a lesser extent (49.4±19.1 mm³; P<0.05). Importantly, the disruption of early steps of platelet activation was not accompanied by an increase in bleeding complications as revealed by serial magnetic resonance imaging. In contrast, blockade of the final common pathway of platelet aggregation with anti-GPIIb/IIIa F(ab)₂ fragments had no positive effect on stroke size and functional outcome but increased the incidence of intracerebral hemorrhage and mortality after transient middle cerebral artery occlusion in a dose-dependent manner. Our data indicate that the selective blockade of key signaling pathways of platelet adhesion and aggregation has a different impact on stroke outcome and bleeding complications. Inhibition of early steps of platelet adhesion to the ischemic endothelium and the subendothelial matrix may offer a novel and safe treatment strategy in acute stroke.

It is known that reduction of t-PA activity (t-PA gene inactivation or PAI-1 gene transfer) reduces cerebral infarct size, while its augmentation (t-PA gene transfer or PAI-1 gene inactivation) increases infarct size (Y. F. Wang et al., Nature Medicine 1998, 4:228-231). Moreover, t-PA has a neurotoxic effect on persistent focal cerebral ischemia, which also occurs with other thrombolytic agents, including streptokinase and staphylokinase.

On the other hand, the reduction of plasminogen activity (Plg gene inactivation or α2-AP injection) increases cerebral infarct size, while its augmentation (α2-AP gene inactivation or α2-AP neutralization) reduces the cerebral infarct size. Moreover, it is known from Guy L. Reed et al., WO1998012329 1998 Jul. 23, that stroke can be treated by using alpha-2-antiplasmin (α 2-AP) binding compounds such as antibodies to alpha-2-antiplasmin to enhancing fibrinolysis. Johann Eibl et al., U.S. Pat. No. 6,114,506 1998 Jul. 17, described a treatment of ischemic events, including cerebral ischemia, and reperfusion injury associated with ischemic events by administration of plasmin and plasmin-forming proteins, including lys-plasminogen and similar substances in a manner that avoids or minimizes the adverse effects associated with conventional treatments, such as reperfusion injury. These findings have suggested two independent (t-PA-mediated and Plg-mediated, respectively) mechanisms operating in opposite direction. In view of the above, the use of the ligands that inhibit GPIb functions can be combined with compounds that increase α2-AP activity for the treatment of transient cerebral attacks that develop to stroke or focal cerebral ischemic infarction (ischemic stroke) or cerebral attacks of the stroke or focal cerebral ischemic infarction (ischemic stroke) type.

In a specific embodiment of the invention, the anti-GPIb antibodies, but preferably, monovalent antibodies such as a monoclonal Fab fragment or a ScFv comprising both a heavy chain variable domain and/or a light chain variable domain or antibody fragments comprising a variable domain, such as a heavy chain variable domain and/or a light chain variable domain or antibody fragments comprising only the variable domain, such as a heavy chain variable domain and/or a light chain variable domain, for treatment of a cerebral infarct such as a transient cerebral attack, in particular, a stroke or an ischemic stroke, are combined with an alpha-2-antiplasmin (α 2-AP) binding compounds such as antibodies to alpha-2-antiplasmin, human plasmin, human plasmin-forming proteins, including lys-plasminogen or similar substances to enhance fibrinolysis, e.g., by lowering the concentration of α2-AP or lowering its activity.

α2-AP neutralizing antibodies or derivatives thereof, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or single domain antibodies or single domain antibody fragments comprising only the variable domain, such as a heavy chain variable domain and/or a light chain variable domain or compounds that neutralize α2-AP or increase fibrinolysis are, for example, plasmin, mini-plasmin (lacking the first four kringles), micro-plasmin (lacking all five kringles), or human plasmin-forming proteins, including lys-plasminogen or similar substances, are thus suitable in a combined treatment with the anti-GPIb antibodies, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or antibody fragments comprising only the variable domain, such as a heavy chain variable domain and/or a light chain variable domain, for treatment of a cerebral infarct such as a transient cerebral attack, in particular, a stroke or an ischemic stroke.

Growth factor-mediated improved perfusion of the penumbra in the brain or of the jeopardized myocardium of patients suffering ischemic events, either via increased vasodilation or angiogenesis (the formation of endothelial-lined vessels), may be of great therapeutic value according to Isner et al. in J. Clin. Invest. (1999), 103(9):1231-6. Capillary blood vessels consist of endothelial cells and pericytes, which carry all the genetic information required to form tubes, branches and entire capillary networks. Specific angiogenic molecules can initiate this process. A number of polypeptides that stimulate angiogenesis have been purified and characterized as to their molecular, biochemical and biological properties, as reviewed by Klagsbrun et al. in Ann. Rev. Physiol. (1991), 53:217-239, and by Folkman et al. in J. Biol. Chem. (1992), 267:10931-4. One factor that can stimulate angiogenesis and that is highly specific as a mitogen for vascular endothelial cells, is termed “vascular endothelial growth factor” (hereinafter referred as “VEGF”) according to Ferrara et al. in J. Cell. Biochem. (1991) 47:211-218. VEGF is also known as “vasculotropin.” Connolly et al. also describe in J. Biol. Chem. (1989), 264:20017-20024, in J. Clin. Invest. (1989), 84:1470-8, and in J. Cell. Biochem. (1991), 47:219-223, a human vascular permeability factor that stimulates vascular endothelial cells to divide in vitro and promotes the growth of new blood vessels when administered into healing rabbit bone grafts or rat corneas. The term “vascular permeability factor” (“VPF” for abbreviation) was adopted because of increased fluid leakage from blood vessels following intradermal injection and appears to designate the same substance as VEGF. The murine VEGF gene has been characterized and its expression pattern in embryogenesis has been analyzed.

A persistent expression of VEGF was observed in epithelial cells adjacent to fenestrated endothelium, e.g., in chloroid plexus and kidney glomeruli, which is consistent with its role as a multifunctional regulator of endothelial cell growth and differentiation as disclosed by Breier et al. in Development (1992), 114:521-532. VEGF shares about 22% sequence identity, including a complete conservation of eight cysteine residues, according to Leung et al. in Science (1989), 246:1306-9, with human platelet-derived growth factor PDGF, a major growth factor for connective tissue. Alternatively, spliced mRNAs have been identified for both VEGF and PDGF and these splicing products differ in their biological activity and receptor-binding specificity.

VEGF is a potent vasoactive protein that has been detected in and purified from media conditioned by a number of cell lines including pituitary cells, such as bovine pituitary follicular cells (as disclosed by Ferrara et al. in Biochem. Biophys. Res. Comm. (1989), 161:851-858, and by Gospodarowicz et al. in Proc. Natl. Acad. Sci. USA (1989), 86:7311-5), rat glioma cells (as disclosed by Conn et al. in Proc. Natl. Acad. Sci. USA (1990), 87:1323-1327) and several tumor cell lines. Similarly, an endothelial growth factor isolated from mouse neuroblastoma cell line NB41 with an unreduced molecular mass of 43-51 kDa has been described by Levy et al. in Growth Factors (1989), 2:9-19. VEGF was characterized as a glycosylated cationic 46 kDa dimer made up of two sub-units each with an apparent molecular mass of 23 kDa. It is inactivated by sulfhydryl-reducing agents, resistant to acidic pH and to heating, and binds to immobilized heparin.

VEGF has four different forms of 121, 165, 189 and 206 amino-acids due to alternative splicing of mRNA. The various VEGF species are encoded by the same gene. Analysis of genomic clones in the area of putative mRNA splicing also shows an intron/exon structure consistent with alternative splicing. The VEGF165 species is the molecular form predominantly found in normal cells and tissues. The VEGF 121 and VEGF165 species are soluble proteins and are capable of promoting angiogenesis, whereas the VEGF 189 and VEGF206 species are mostly cell-associated. All VEGF isoforms are biologically active, e.g., each of the species when applied intradermally is able to induce extravasation of Evans blue. However, VEGF isoforms have different biochemical properties that may possibly modulate the signaling properties of the growth factors.

The VEGF 165, VEGF 189 and VEGF206 species contain eight additional cysteine residues within the carboxy-terminal region. The amino-terminal sequence of VEGF is preceded by 26 amino acids corresponding to a typical signal sequence. The mature protein is generated directly following signal sequence cleavage without any intervening prosequence. Other VEGF polypeptides from the PDGF family of growth factors have been disclosed in U.S. Pat. No. 5,840,693. Purified and isolated VEGF-C cysteine deletion variants that bind to a VEGF tyrosine kinase receptor have been disclosed in U.S. Pat. No. 6,130,071. Like other cytokines, VEGF can have diverse effects that depend on the specific biological context in which it is found.

The expression of VEGF is high in vascularized tissues (e.g., lung, heart, placenta and solid tumors) and correlates with angiogenesis, both temporally and spatially. VEGF has been shown to directly contribute to induction of angiogenesis in vivo by promoting endothelial cell growth during normal embryonic development, wound healing, tissue regeneration and reorganization. Therefore, VEGF has been proposed for use in promoting vascular tissue repair, as disclosed by EP-A-0,506,477. VEGF is also involved in pathological processes such as growth and metastasis of solid tumors and ischemia-induced retinal disorders such as disclosed in U.S. Pat. No. 6,114,320. VEGF expression is triggered by hypoxia so that endothelial cell proliferation and angiogenesis appear to be especially stimulated in ischemic areas.

Finally, U.S. Pat. No. 6,040,157 discloses human VEGF2 polypeptides that have been putatively identified as novel vascular endothelial growth factors based on their amino acid sequence homology to human VEGF. The latter document further discloses restoration of certain parameters in the ischemic limb by using a VEGF2 protein. However, it is also known by Hariawala et al. in J. Surg. Res. (1996), 63(1):77-82, that a systemic administration of VEGF, in high doses over short periods of time, improves myocardial blood flow but produces hypotension in porcine hearts. Placenta growth factor (hereinafter referred as “PIGF”) was disclosed by Maglione et al. in Proc. Natl. Acad. Sci. USA (1991), 88(20):9267-71, as a protein related to the vascular permeability factor. U.S. Pat. No. 5,919,899 discloses nucleotide sequences coding for a protein, namely PIGF, which can be used in the treatment of inflammatory diseases and in the treatment of wounds or tissues after surgical operations, transplantations, burns, or ulcers, and so on. Soluble non-heparin-binding and heparin-binding forms, built up of 131 and 152 amino acids, respectively, have been described for PIGF, which is expressed in placenta, trophoblastic tumors and cultured human endothelial cells, according to U.S. Pat. No. 5,776,755.

Bohlen et al. WO9849300 found that VEGF levels are increased in response to ischemia and that therapy using VEGF and related factors is useful for stroke. Bohlen et al. also describes that treatment by VEGF or VEGF/PLGF heterodimers and truncated forms results in a relaxation of arteries, which is beneficial on decreasing infarct size.

Growth factors for mediated improved perfusion such as VEGF, PLGF or VEGF/PLGF heterodimers are thus suitable in a combined treatment with the anti-GPIb antibodies, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or antibody fragments comprising only the variable domain, such as a heavy chain variable domain and/or a light chain variable domain, for treatment of a cerebral infarct such as a transient cerebral attack, in particular, a stroke or an ischemic stroke.

Bolus intravenous anti-GPIb monovalent antibodies given one hour after thrombotic MCA occlusion in mice reduces cerebral ischemic damage and improves neurological dysfunction, suggesting that anti-GPIb can be beneficial in ischemic stroke patients. α₂-AP is known to increase the focal ischemic cerebral infarct size and reduction of α₂-AP is known to inhibit such infarct size. The anti-GPIb treatment of focal cerebral ischemic infarction, according to the invention, can further be combined with an α2-antiplasmin neutralizing antibody or with plasmin or its derivatives, mini-plasmin (lacking the first four kringles), and micro-plasmin (lacking all five kringles), e.g., by a simultaneous or separate bolus injection.

The invention is broadly drawn to a treatment of occlusive syndrome in the cerebral vascular system or of cerebral thrombosis resulting into cerebral infarction, such as (ischemic) stroke and protecting such subject from ischemic brain injury in a stroke without an increase in bleeding complications by inhibiting or preventing platelet collagen signaling through GPVI or platelet VWF-induced signaling through GPIb-IX-V.

Another aspect of the invention is a cell line deposited with the Belgian Coordinated Collections of Microorganisms, under accession number LMBP 5108CB. The cell line can be a cell line that is producing monoclonal antibodies having a reactivity substantially identical to that of the monoclonal antibodies obtained from the LMBP 5108CB. A further aspect of the invention is a ligand derived from a monoclonal antibody obtainable from the LMBP 5108CB cell line. This ligand is characterized in that it prevents the binding of von Willebrand factor to the human platelet glycoprotein GPIb, e.g., by binding to the human platelet glycoprotein GPIb and that it prevents the binding of von Willebrand factor to the human platelet glycoprotein GPIb. The ligand does not produce thrombocytopenia when administered to a primate at a dose of up to at least 4 mg/kg by bolus intravenous administration.

One aspect of the inventions a ligand that binds to the human platelet glycoprotein GPIb and prevents the binding of von Willebrand factor to human GPIb. Such ligand can be characterized in that it does not produce thrombocytopenia when administered to a primate at a dose of up to at least 4 mg/kg by bolus intravenous administration.

In a specific embodiment of the invention, the above-described ligand is an antibody, more specifically, a monovalent antibody. In yet another aspect of the invention, such an antibody ligand is a Fab fragment of the monoclonal antibody. This antibody, specifically Fab fragment, is able to recognize an epitope located on human platelet glycoprotein GPIb. In yet another embodiment of the invention, this Fab fragment is derived from a monoclonal antibody produced by intentional immunization in animals. The invention thus also concerns a humanized or hybridized monoclonal antibody derivable from this monoclonal antibody that is derivable from the cell line LMBP 5108CB. Furthermore, the invention concerns an antigen-binding Fab fragment or a homolog or derivative of the above-described monoclonal antibody derived from the cell line LMBP 5108CB.

Yet another aspect of the invention is a pharmaceutical composition comprising a ligand that prevents the binding of von Willebrand factor to the human platelet glycoprotein GPIb, e.g., by binding to the human platelet glycoprotein GPIb, a humanized or hybridized monoclonal antibody that prevents the binding of von Willebrand factor to the human platelet glycoprotein GPIb, e.g., by binding to the human platelet glycoprotein GPIb, and that it prevents the binding of von Willebrand factor to the human platelet glycoprotein GPIb, e.g., a humanized or hybridized monoclonal antibody derivable from this monoclonal antibody that is derivable from the cell line LMBP 5108CB or an antigen-binding Fab fragment of such antibody as described above in admixture with a pharmaceutically acceptable carrier.

Such pharmaceutical carrier may further comprise a thrombolytic agent in a form either for simultaneous or sequential use. Such thrombolytic agent can, e.g., be α2-AP neutralizing antibodies or derivatives thereof, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or single domain antibodies or single domain antibody fragments comprising only the variable domain, such as a heavy chain variable domain and/or a light chain variable domain or compounds that neutralize α2-AP or increase fibrinolysis that are, for example, plasmin, mini-plasmin (lacking the first four kringles), micro-plasmin (lacking all five kringles), or human plasmin-forming proteins, including lys-plasminogen or similar substances, and are thus suitable in a combined treatment with the anti-GPIb antibodies, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or antibody fragments comprising only the variable domain, such as a heavy chain variable domain and/or a light chain variable domain for treatment of a cerebral infarct such as a transient cerebral attack, in particular, a stroke or an ischemic stroke.

This humanized or hybridized monoclonal antibody or the antigen-binding Fab fragment that prevents the binding of von Willebrand factor to the human platelet GPIb, e.g., by binding to the human platelet GPIb as described above, may be used as a medicament. The use of humanized or hybridized monoclonal antibody or the antigen-binding Fab fragment that prevents the binding of von Willebrand factor to the human platelet GPIb, e.g., by binding to the human platelet GPIb as described above, can be in simultaneous or sequential association with at least a thrombolytic agent, such as α2-AP neutralizing antibodies or derivatives thereof, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or single domain antibodies or single domain antibody fragments comprising only the variable domain, such as a heavy chain variable domain and/or a light chain variable domain or compounds that neutralize α2-AP or increase fibrinolysis that are, for example, plasmin, mini-plasmin (lacking the first four cringles), micro-plasmin (lacking all five cringles), or human plasmin-forming proteins, including lys-plasminogen or similar substances, are thus suitable in a combined treatment with the anti-GPIb antibodies, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or antibody fragments comprising only the variable domain, such as a heavy chain variable domain and/or a light chain variable domain, for treatment of a cerebral infarct such as a transient cerebral attack, in particular, a stroke or an ischemic stroke. This humanized or hybridized monoclonal antibody or the antigen-binding Fab fragment that prevents the binding of von Willebrand factor to the human platelet GPIb, e.g., by binding to the human platelet GPIb, as described above, can be used for the treatment and/or prevention of a disorder of hemostasis, in particular, an occlusive syndrome in the cerebral vascular system or of cerebral thrombosis resulting into transient cerebral infarct such as stroke, ischemic stroke or acute stroke.

Also disclosed is the use of humanized or hybridized monoclonal antibody or the antigen-binding Fab fragment that prevents the binding of von Willebrand factor to the human platelet glycoprotein GPIb, e.g., by binding to the human platelet glycoprotein GPIb as described above, wherein the medicament is for oral, intranasal, subcutaneous, intramuscular, intradermal, intravenous, intra-arterial or parenteral administration or for catheterization.

Also disclosed is a polynucleotide encoding for an antigen-binding Fab fragment as described above that prevents the binding of von Willebrand factor to the human platelet glycoprotein GPIb, e.g., by binding to the human platelet glycoprotein GPIb. The invention may further comprise a polynucleotide sequence of the above-described polynucleotide encoding for an antigen-binding Fab fragment comprising a nucleic acid molecule having a sequence complementary to the coding sequence of the polynucleotide. These polynucleotide sequences can be of the group of the sequences a shown in SEQ ID NO:1, as shown in SEQ ID NO:2, as shown in SEQ ID NO:3 or as shown in SEQ ID NO:4.

Still another aspect of the invention is a pharmaceutical composition comprising a monovalent antibody fragment that prevents the binding of von Willebrand factor (vWF) to human platelet GPIb and binds in vivo to human platelet GPIb without incurring thrombocytopenia, and a pharmaceutically acceptable carrier, wherein the variable region of the fragment comprises SEQ ID NO:4. Also disclosed is a pharmaceutical composition comprising a monovalent antibody fragment that prevents the binding of vWF to human platelet GPIb and binds in vivo to human platelet GPIb without incurring thrombocytopenia, and a pharmaceutically acceptable carrier, wherein the monovalent antibody fragment is obtained from a monoclonal antibody produced by the cell line deposited with the Belgian Coordinated Collections of Microorganisms, under accession number LMBP 5108CB.

Another aspect of present invention is a monoclonal antibody produced by the cell line deposited with the Belgian Coordinated Collections of Microorganisms, under accession number LMBP 5108CB.

The invention also comprises a cell line capable of producing an antibody directed against GPIb deposited with the Belgian Coordinated Collections of Microorganisms, under accession number LMBP 5108CB. Such antibody can be a humanized antibody fragment derivable from the monoclonal antibody, wherein the humanized antibody fragment binds GPIb.

Another object of the invention is a monovalent antibody fragment that binds in vivo to human platelet GPIb and prevents the binding of von Willebrand factor to human platelet GPIb, wherein the monovalent antibody fragment is obtained from a monoclonal antibody produced by the cell line deposited with the Belgian Coordinated Collections of Microorganisms under accession number LMBP 5108CB.

Yet another embodiment of the invention is a monovalent antibody fragment that binds in vivo to human platelet GPIb and prevents the binding of von Willebrand factor to human platelet GPIb, wherein the monovalent antibody fragment includes a variable region comprising SEQ ID NO:4.

Also disclosed is a pharmaceutical composition comprising a fragment of a monoclonal antibody being able to bind to human platelet glycoprotein GPIb and preventing the binding of von Willebrand factor to human platelet glycoprotein GPIb, in admixture with a pharmaceutically acceptable carrier, characterized in that the fragment is a Fab fragment. Such pharmaceutical composition can be characterized in that the Fab fragment is a Fab fragment of a monoclonal antibody obtainable from the cell line deposited with the Belgian Coordinated Collections of Microorganisms, under accession number LMBP 5108CB. In this pharmaceutical composition, the Fab fragment can be a fragment that inhibits platelet adhesion at a shear rate of between 650 and 2,600 s⁻¹. In a particular aspect of the invention, such pharmaceutical composition comprising a fragment of a monoclonal antibody being able to bind to human platelet glycoprotein GPIb and preventing the binding of von Willebrand factor to human platelet glycoprotein GPIb or a Fab fragment thereof does not produce thrombocytopenia when administered to a primate at a dose of up to at least 4 mg/kg by bolus intravenous administration. The monoclonal antibody herein is, in a particular embodiment, produced by intentional immunization in animals or it is a Fab fragment that is a humanized Fab fragment. Such a pharmaceutical composition can further comprise a therapeutically effective amount of a thrombolytic agent such as α2-AP neutralizing antibodies or derivatives thereof, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or single domain antibodies or single domain antibody fragments comprising only the variable domain, such as a heavy chain variable domain and/or a light chain variable domain or compounds that neutralize α2-AP or increase fibrinolysis, which are, for example, plasmin, mini-plasmin (lacking the first four kringles), micro-plasmin (lacking all five kringles), or human plasmin-forming proteins, including lys-plasminogen or similar substances.

In a specific embodiment of the invention, the pharmaceutical composition comprising a fragment of a monoclonal antibody being able to bind to human platelet glycoprotein GPIb and preventing the binding of von Willebrand factor to human platelet glycoprotein GPIb, in admixture with a pharmaceutically acceptable carrier, further comprising a therapeutically effective amount of aspirin or heparin and such composition, can be for the prevention or treatment of a hemostasis disorder, in particular, a hemostasis disorder that is an occlusive syndrome in the cerebral vascular system or a cerebral thrombosis resulting in transient cerebral infarct such as (ischemic) stroke. This pharmaceutical composition can be used to protect a subject from ischemic brain injury in a stroke without an increase in bleeding complications.

In a specific embodiment of the invention, the pharmaceutical composition comprising a fragment of a monoclonal antibody being able to bind to human platelet glycoprotein GPIb and preventing the binding of von Willebrand factor to human platelet glycoprotein GPIb, in admixture with a pharmaceutically acceptable carrier, further comprising a therapeutically effective amount of a thrombolytic agent and such composition, can be for use as an anti-thrombotic, in particular, to treat an occlusive syndrome in the cerebral vascular system or a cerebral thrombosis, which, if untreated, results in transient cerebral infarct such as (ischemic) stroke. This pharmaceutical composition can be used to protect a subject from ischemic brain injury in a stroke without an increase in bleeding complications. This pharmaceutical composition can be for simultaneous or sequential association with a thrombolytic agent.

Also disclosed is a pharmaceutical composition comprising a fragment of a fab fragment of a monoclonal antibody being able to bind to human platelet glycoprotein GPIb and preventing the binding of von Willebrand factor to human platelet glycoprotein GPIb, in admixture with a pharmaceutically acceptable carrier, characterized in that the VI region of the Fab fragment is encoded by a sequence comprising SEQ ID NO: 1.

Also disclosed is a pharmaceutical composition comprising a fragment of a fab fragment of a monoclonal antibody being able to bind to human platelet glycoprotein GPIb and preventing the binding of von Willebrand factor to human platelet glycoprotein GPIb, in admixture with a pharmaceutically acceptable carrier, characterized in that the Vh region of the Fab fragment is encoded by a sequence comprising SEQ ID NO:2.

Also disclosed is a pharmaceutical composition comprising a fragment of a fab fragment of a monoclonal antibody being able to bind to human platelet glycoprotein GPIb and preventing the binding of von Willebrand factor to human platelet glycoprotein GPIb, in admixture with a pharmaceutically acceptable carrier, characterized in that the VI region of the Fab fragment is encoded by a sequence comprising SEQ ID NO:3.

Also disclosed is a pharmaceutical composition comprising a fragment of a fab fragment of a monoclonal antibody being able to bind to human platelet glycoprotein GPIb and preventing the binding of von Willebrand factor to human platelet glycoprotein GPIb, in admixture with a pharmaceutically acceptable carrier, characterized in that the Vh region of the Fab fragment is encoded by a sequence comprising SEQ ID NO:4.

Also disclosed is a pharmaceutical composition comprising a fragment of a monoclonal antibody being able to bind to human platelet glycoprotein GPIb and capable of preventing the binding of von Willebrand factor to human platelet glycoprotein GPIb, for use as a medicament, wherein the fragment is a Fab fragment. This fragment of this monoclonal antibody can be a fragment of a monoclonal antibody produced by the cell line deposited with the Belgian Coordinated Collections of Microorganisms, under accession number LMBP 5108CB.

Also disclosed is a method for treating a subject or patient suffering from or at risk of developing a platelet-dependent disorder that comprises administering to the patient a therapeutically effective amount of an inhibitory anti-GPIb monovalent antibody fragment that binds in vivo to human platelet GPIb and prevents the binding of von Willebrand factor to the human platelet GPIb, wherein administration of the monovalent antibody fragment does not incur thrombocytopenia in the patient. In a particular embodiment, this platelet-dependent disorder is an occlusive syndrome in the cerebral vascular system or a cerebral thrombosis, which, if untreated, results into transient cerebral infarct such as (ischemic) stroke, wherein the method protects the subject or patient from ischemic brain injury in a stroke without an increase in bleeding complications. The monovalent antibody fragment used in this method is, in a particular embodiment, a Fab fragment or a single variable domain. In particular, such monovalent antibody fragment can be a humanized antibody fragment, or monovalent antibody fragment comprising SEQ ID NO:4, or the monovalent antibody fragment is, in a particular embodiment, an antibody fragment from a monoclonal antibody produced by the cell line deposited with the Belgian Coordinated Collections of Microorganisms, under accession number LMBP 5108CB.

The method of the invention for treating a subject or patients suffering from, or at risk of developing, a platelet-dependent disorder, which comprises, administering to the patient a therapeutically effective amount of an inhibitory anti-GPIb monovalent antibody fragment that binds in vivo to human platelet GPIb and prevents the binding of von Willebrand factor to the human platelet GPIb, wherein administration of the monovalent antibody fragment does not incur thrombocytopenia in the patient, and can be for a platelet-dependent disorder that is a hemostasis disorder. Such hemostasis disorder is, e.g., an occlusive syndrome in the cerebral vascular system or a cerebral thrombosis resulting in transient cerebral infarct such as (ischemic) stroke, in which case, the treatment protects a subject from ischemic brain injury in a stroke without an increase in bleeding complications.

The method of the invention for treating a subject or patient suffering from or at risk of developing a platelet-dependent disorder comprises administering to the patient a therapeutically effective amount of an inhibitory anti-GPIb monovalent antibody fragment that binds in vivo to human platelet GPIb and prevents the binding of von Willebrand factor to the human platelet GPIb, wherein administration of the monovalent antibody fragment does not incur thrombocytopenia in the patient, can be for a platelet-dependent disorder that is a platelet-dependent thrombus formation. Such platelet-dependent thrombus formation is, e.g., an occlusive syndrome in the cerebral vascular system or a cerebral thrombosis resulting in transient cerebral infarct such as (ischemic) stroke, in which case the treatment protects a subject from ischemic brain injury in a stroke without an increase in bleeding complications.

The method of the invention for treating a subject or patient suffering from, or at risk of developing, a platelet-dependent disorder can further comprise administering to the patient, simultaneously or sequentially, a thrombolytic agent.

The method of the invention for treating a subject or patient suffering from, or at risk of developing, a platelet-dependent disorder comprises administering to the patient a therapeutically effective amount of an inhibitory anti-GPIb monovalent antibody fragment that binds in vivo to human platelet GPIb and prevents the binding of von Willebrand factor to the human platelet GPIb, wherein administration of the monovalent antibody fragment does not incur thrombocytopenia in the patient for a platelet-dependent disorder, and can further comprise administering to the patient in adjunctive therapy, one or more other anti-thrombotic agents. Such other anti-thrombotic agent can be aspirin or heparin.

The method of the invention for treating a subject or patient suffering from, or at risk of developing, a platelet-dependent disorder comprises administering to the patient a therapeutically effective amount of an inhibitory anti-GPIb monovalent antibody fragment that binds in vivo to human platelet GPIb and prevents the binding of von Willebrand factor to the human platelet GPIb, wherein administration of the monovalent antibody fragment does not incur thrombocytopenia in the patient for a platelet-dependent disorder and can further comprise administering to the patient in adjunctive therapy, a placenta growth factor (PIGF), a fragment, a derivative or a homologue thereof, or a vascular endothelial growth factor (VEGF), a fragment, a derivative or a homologue thereof, or a combination of PIGF and VEGF, or a VEGF/PIGF heterodimer.

Also disclosed is a method of treatment of an occlusive syndrome in the cerebral vascular system or a cerebral thrombosis resulting in transient cerebral infarct, such as (ischemic) stroke, by a pharmaceutical composition comprising a ligand that is an antibody or an antigen-recognizing fragment thereof, binding specifically vWF and inhibiting the interaction of vWF with the GPIb/V/IX complex.

Also disclosed is a method of treatment of an occlusive syndrome in the cerebral vascular system or a cerebral thrombosis resulting in transient cerebral infarct such as (ischemic) stroke by a pharmaceutical composition comprising a ligand that is an antibody or an antigen-recognizing fragment thereof binding specifically to the A1 domain of vWF.

Also disclosed is a ligand that is an antibody or an antigen-recognizing fragment thereof binding specifically to the A3 domain of von Willebrand Factor (vWF) or an epitope thereof for use as a medicament. Such ligand can be further characterized in that it binds specifically to an epitope comprising amino acids located within the sequence spanning amino acids 974 to 989 within the A3 domain of vWF or that it binds to an epitope comprising amino acids PW (aa 981-982) within the A3 domain of vWF or that it binds to an epitope comprising amino acids S, P, W and R within the A3 domain of vWF or that it does not block the GPIb-vWF binding or GPIIb-IIIa receptor binding.

Also disclosed is a ligand that is an antibody or an antigen-recognizing fragment thereof binding specifically to the A3 domain of von Willebrand Factor (vWF) or an epitope thereof for use as a medicament, which is further characterized in that when the ligand is administered to a baboon by bolus intravenous administration at a dose corresponding to 300 microgram/kg of monoclonal antibody, it inhibits vWF binding to collagen at least up to five hours after injection or further characterized in that the ligand ensures that it does not induce a severe decline in circulating vWF-levels or a severe drop in platelet count when the ligand is administered to a primate by bolus intravenous administration at a dose up to 600 microgram/kg or further characterized in that the ligand ensures that bleeding time remains unchanged or that thrombocytopenia is not induced when the ligand is administered to a primate by bolus intravenous administration at a dose up to 600 microgram/kg or further characterized in that the ligand induces vWF-occupancy and inhibits vWF-collagen binding when administered at a therapeutically effective dose up to 600 microgram/kg to a primate by bolus intravenous administration.

Also disclosed is a ligand that is an antibody or an antigen-recognizing fragment thereof binding specifically to the A3 domain of von Willebrand Factor (vWF) or an epitope thereof for use as a medicament, which is further characterized in that clotting time (Prothrombin Time (FT) or activated Partial Thromboplastin Time (aPTT)) remains unaffected, and vWF-collagen binding is inhibited and induces increased vWF-occupancy when the ligand is administered to a primate by bolus intravenous administration at a therapeutically effective dose up to 600 microgram/kg or further characterized in that at a concentration of 1 μg/ml, it completely inhibits platelet deposition on a collagen substrate at a shear rate of 1300 s⁻¹ or higher.

Also disclosed is a ligand that is an antibody or an antigen-recognizing fragment thereof binding specifically to the A3 domain of von Willebrand Factor (vWF) or an epitope thereof for use as a medicament that, when administered to an individual as an anti-thrombotic agent, inhibits interaction of vWF with collagen and does not induce severe bleeding disorders at a minimal medicinal effective dose to exhibit anti-thrombotic action.

Also disclosed is a ligand that is an antibody or an antigen-recognizing fragment thereof binding specifically to the A3 domain of von Willebrand Factor (vWF) or an epitope thereof for use as a medicament that, when administered to an individual as an anti-thrombotic agent, maintains circulating vWF levels or platelet counts at a minimal medicinal dose effective to exhibit anti-thrombotic action.

The ligand to vWF, according to any of the previous embodiments, can be a monoclonal antibody deposited with the Belgian Collections of Microorganisms under accession number LMBP 5606CB or an antigen-recognizing fragment thereof.

Furthermore, the invention concerns immunoconjugate comprising the ligand of vWF as described in the previous embodiments and a thrombolytic agent. Such thrombolytic agent can be a α2-AP-neutralizing antibody or a derivative thereof, preferably a monovalent antibody such as a monoclonal Fab fragment or a ScFv comprising both a heavy chain variable domain and/or a light chain variable domain of antibody fragments comprising a variable domain, such as a heavy chain variable domain and/or a light chain variable domain or single domain antibodies or single domain antibody fragments comprising only the variable domain, such as a heavy chain variable domain and/or a light chain variable domain or compounds that neutralize α2-AP or increase fibrinolysis, are, for example, plasmin, mini-plasmin (lacking the first four kringles), micro-plasmin (lacking all five kringles), or human plasmin-forming proteins, including lys-plasminogen or similar substances.

Also disclosed is a pharmaceutical composition comprising the ligand of vWF or its immunoconjugate of the previous embodiments in admixture with a pharmaceutically acceptable carrier.

A particular embodiment of the invention is a method of anti-thrombotic therapy in an individual, comprising administering to the individual at risk of occlusive syndrome in the cerebral vascular system or cerebral thrombosis, a therapeutically effective amount of an antibody, or an antigen-recognizing fragment thereof, binding to von Willebrand factor. Preferably, this ligand does not block GPIIb-IIIa receptor binding and such ligand does not induce a severe bleeding disorder at minimal medicinal effective dose to exhibit the anti-thrombotic action on the occlusive syndrome in the cerebral vascular system or cerebral thrombosis. Such ligand can also be a monovalent antibody, such as a monoclonal Fab, a fab′ fragment or a ScFv comprising both a heavy chain variable domain and/or a light chain variable domain of antibody fragments comprising a variable domain, such as a heavy chain variable domain and/or a light chain variable domain or a single domain antibody fragment comprising only the variable domain, such as a heavy chain variable domain and/or a light chain variable domain for treatment of a cerebral infarct such as a transient cerebral attack, in particular, a stroke or an ischemic stroke. The ligand can be a full human antibody or a humanized antibody having only the hypervariable region of non-human origin. In one embodiment, the ligand is an IgG, Fab, Fab′ or a F(ab′)2. The method of treatment of occlusive syndrome in the cerebral vascular system or cerebral thrombosis may further comprise simultaneously or sequentially to the individual a thrombolytic agent, e.g., ligands that bind to von Willebrand factor may be combined with α2-AP-neutralizing antibodies or derivatives thereof, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or single domain antibodies or single domain antibody fragments comprising only the variable domain, such as a heavy chain variable domain and/or a light chain variable domain or compounds that neutralize α2-AP or increase fibrinolysis, are, for example, plasmin, mini-plasmin (lacking the first four kringles), micro-plasmin (lacking all five kringles), or human plasmin-forming proteins, including lys-plasminogen or similar substances.

A certain embodiment of the invention concerns treatment of a subject suffering from, or at risk of, occlusive syndrome in the cerebral vascular system, of transient cerebral attacks or of cerebral thrombosis resulting in cerebral infarction referred to as stroke, ischemic stroke or acute stroke, or by a monovalent antibody against platelet glycoprotein GPIb that inhibits or prevents the activation of GPIb-mediated pathways leading to thrombus formation in the cerebral vascular system, e.g., by a monovalent antibody against platelet glycoprotein GPIb that inhibits or prevents activation of a GPIB-mediated pathway by preventing the interaction with its naturally activating ligands such as von Willebrand factor, P-selectin, or MAC-1 to protect a subject from ischemic brain injury in a stroke without an increase in bleeding complications by inhibiting signaling responses to collagen and inhibiting initial adhesion/attachment of platelets to endothelial cells.

Particular embodiments of the invention include the following recitations:
1. Cell line deposited with the Belgian Coordinated Collections of Microorganisms under accession number LMBP 5108CB.
2. A cell line producing monoclonal antibodies having a reactivity substantially identical to that of the monoclonal antibodies obtained from this cell line.
3. A ligand that binds to the human platelet glycoprotein GPIb and prevents the binding of von Willebrand factor to human GPIb.
4. A ligand that does not produce thrombocytopenia when administered to a primate at a dose of up to at least 4 mg/kg by bolus intravenous administration.
5. A ligand derived from a monoclonal antibody obtainable from the cell lines of recitation 1 or recitation 2.
6. A ligand according to recitation 5 that binds to the human platelet glycoprotein GPIb.
7. A ligand according to recitation 5 or recitation 6 that prevents the binding of von Willebrand factor to the human platelet glycoprotein GPIb.
8. A ligand according to any of recitations 5 to 7 that does not produce thrombocytopenia when administered to a primate at a dose of up to at least 4 mg/kg by bolus intravenous administration.
9. A ligand according to any of recitations 5 to 8 being a Fab fragment of the monoclonal antibody.
10. A ligand according to any of recitations 5 to 9 being able to recognize an epitope located on human platelet glycoprotein GPIb.
11. A ligand according to any of recitations 3 to 9 and being derived from a monoclonal antibody produced by intentional immunization in animals.
12. A humanized or hybridized monoclonal antibody derivable from the monoclonal antibody of recitation 11 or derivable from the cell lines of recitations 1 or 2.
13. An antigen-binding Fab fragment or a homolog or derivative of a monoclonal antibody according to recitations 11 or 12 or derived from the cell lines of recitations 1 or 2.
14. A pharmaceutical composition comprising a ligand according to any of recitations 3 to 11, a humanized or hybridized monoclonal antibody according to recitation 12 or an antigen-binding Fab fragment according to recitation 13, in admixture with a pharmaceutically acceptable carrier.
15. A pharmaceutical composition according to recitation 14, further comprising a thrombolytic agent in a form either for simultaneous or sequential use.
16. Use of the ligand according to any of recitations 3 to 11, the humanized or hybridized monoclonal antibody of recitation 12 or an antigen-binding Fab fragment of recitation 13 as a medicament.
17. Use according to recitation 16 in simultaneous or sequential association with at least a thrombolytic agent.
18. Use according to recitation 16 or recitation 17 for the treatment and/or prevention of a disorder of hemostasis.
19. Use according to any of recitations 16 to 18, wherein the medicament is for oral, intranasal, subcutaneous, intramuscular, intradermal, intravenous, intra-arterial or parenteral administration or for catheterization.
20. A polynucleotide encoding for an antigen-binding Fab fragment according to recitation 13.
21. A DNA probe for detecting the polynucleotide sequence of recitation 20, comprising a nucleic acid molecule having a sequence complementary to the coding sequence of the polynucleotide.
22. A polynucleotide sequence as shown in SEQ ID NO: 1.
23. A polynucleotide sequence as shown in SEQ ID NO:2.
24. An amino acid sequence as shown in SEQ ID NO:3.
25. An amino acid sequence as shown in SEQ ID NO:4.

Particular embodiments of the invention include following recitations:
1A. A pharmaceutical composition comprising a monovalent antibody fragment that prevents the binding of von Willebrand factor (vWF) to human platelet glycoprotein Ib (GPIb) and binds in vivo to human platelet GPIb without incurring thrombocytopenia, and a pharmaceutically acceptable carrier, wherein the variable region of the fragment comprises SEQ ID NO:4.
2A. A pharmaceutical composition comprising a monovalent antibody fragment that prevents the binding of von Willebrand factor (vWF) to human platelet glycoprotein Ib (GPIb) and binds in vivo to human platelet GPIb without incurring thrombocytopenia, and a pharmaceutically acceptable carrier, wherein said monovalent antibody fragment is obtained from a monoclonal antibody produced by the cell line deposited with the Belgian Coordinated Collections of Microorganisms under accession number LMBP 5108CB.
3A. A monoclonal antibody produced by the cell line deposited with the Belgian Coordinated Collections of Microorganisms under accession number LMBP 5108CB.
4A. A cell line capable of producing an antibody directed against GPIb deposited with the Belgian Coordinated Collections of Microorganisms under accession number LMBP 5108CB.
5A. A humanized antibody fragment derivable from the monoclonal antibody of recitation 3A, wherein the humanized antibody fragment binds GPIb.
6A. A monovalent antibody fragment that binds in vivo to human platelet GPIb and prevents the binding of von Willebrand factor to human platelet GPIb, wherein the monovalent antibody fragment is obtained from a monoclonal antibody produced by the cell line deposited with the Belgian Coordinated Collections of Microorganisms under accession number LMBP 5108CB.
7A. A monovalent antibody fragment that binds in vivo to human platelet GPIb and prevents the binding of von Willebrand factor to human platelet GPIb, wherein the monovalent antibody fragment includes a variable region comprising SEQ ID NO:4.

Particular embodiments of the invention include the following recitations:
1B. A pharmaceutical composition comprising a fragment of a monoclonal antibody being able to bind to human platelet glycoprotein GPIb and preventing the binding of von Willebrand factor to human platelet glycoprotein GPIb, in admixture with a pharmaceutically acceptable carrier, characterized in that the fragment is a Fab fragment.
2B. The pharmaceutical composition of recitation 1B, characterized in that the Fab fragment is a Fab fragment of a monoclonal antibody obtainable from the cell line deposited with the Belgian Coordinated Collections of Microorganisms under accession number LMBP 5108CB
3B. The pharmaceutical composition according to recitation 1B or 2B, wherein the Fab fragment inhibits platelet adhesion at a shear rate of between 650 and 2,600 s⁻¹.
4B. The pharmaceutical composition according to any one of recitations 1B to 3B that does not produce thrombocytopenia when administered to a primate at a dose of up to at least 4 mg/kg by bolus intravenous administration.
5B. The pharmaceutical composition according to any one of recitations 1B to 4B, wherein the monoclonal antibody is produced by intentional immunization in animals.
6B. The pharmaceutical composition according to any one of recitations 1B to 5B, wherein the Fab fragment is a humanized Fab fragment.
7B. The pharmaceutical composition according to any one of recitations 1B to 6B, further comprising a therapeutically effective amount of a thrombolytic agent.
8B. The pharmaceutical composition according to recitation 7B, wherein the thrombolytic agent is selected from compounds that neutralize α2-AP or increase fibrinolysis are, for example, plasmin, mini-plasmin (lacking the first four kringles), micro-plasmin (lacking all five kringles), or human plasmin-forming proteins, including lys-plasminogen or similar substances.
9B. The pharmaceutical composition according to recitations 1B to 6B, further comprising a therapeutically effective amount of aspirin or heparin.
10B. The pharmaceutical composition according to any one of recitations 1B to 9B for the prevention or treatment of a hemostasis disorder.
11B. The pharmaceutical composition according to any one of recitations 1B to 9B for use as an anti-thrombotic.
12B. The pharmaceutical composition according to any one of recitations 1B to 11B for simultaneous or sequential association with a thrombolytic agent.
13B. The pharmaceutical composition according to any one of recitations 1B to 12B, wherein the VI region of the Fab fragment is encoded by a sequence comprising SEQ ID NO:1.
14B. The pharmaceutical composition according to any one of recitations 1B to 12B, wherein the Vh region of the Fab fragment is encoded by a sequence comprising SEQ ID NO:2.
15B. The pharmaceutical composition according to any one of recitations 1B to 14B, wherein the VI region of the Fab fragment is encoded by a sequence comprising SEQ ID NO:3.
16B. The pharmaceutical composition according to any one of recitations 1B to 15B, wherein the Vh region of the Fab fragment is encoded by a sequence comprising SEQ ID NO:4.
17B. A fragment of a monoclonal antibody being able to bind to human platelet glycoprotein GPIb and capable of preventing the binding of von Willebrand factor to human platelet glycoprotein GPIb, for use as a medicament, wherein the fragment is a Fab fragment.
18B. The fragment of a monoclonal antibody of recitation 17, wherein the fragment is a fragment of a monoclonal antibody produced by the cell line deposited with the Belgian Coordinated Collections of Microorganisms under accession number LMBP 5108CB.

Particular embodiments of the invention include any of the following recitations:
1C. A ligand for use as a medicament, wherein the ligand specifically recognizes domain A3 of von Willebrand factor or an epitope of the domain A3.
2C. A ligand against von Willebrand factor (vWF) for use as a medicament, wherein the ligand inhibits interaction of von Willebrand factor with collagen.
3C. The ligand of recitation 1C for use as a medicament, wherein the ligand inhibits interaction of von Willebrand factor with collagen.
4C. The ligand of recitation 2C or 3C for use as a medicament, wherein the collagen is fibrillar collagen fibers.
5C. The ligand of recitation 2C or 3C for use as a medicament, wherein the collagen is thrombogenic collagen.
6C. The ligand of any of recitations 2C to 5C for use as a medicament, wherein the thrombogenic collagen is type I and type III collagen.
7C. The ligand of any of recitations 2C to 6C for use as a medicament, wherein the collagen is exposed in a damaged blood vessel wall
8C. The ligand of any of the recitations 1C to 7C for use as a medicament, wherein the ligand does not directly block the GPIb-vWF axis or the GPIIb-IIIa receptor.
9C. Any of recitations 1C to 8C, wherein the ligand is an antibody.
10C. Any of recitations 1C to 9C, wherein the ligand is an antibody against A3 domain of von Willebrand factor or a fragment thereof.
11C. Any of recitations 1C to 10C, wherein the ligand is a monoclonal antibody or a fragment Fab, Fab′ or F(ab′)2 thereof, or a homologue of the fragment.
12C. Any of recitations 1C to 11C, wherein the ligand is a monoclonal antibody, a fragment Fab, Fab′ or F(ab′)2 thereof, or a homologue of the fragment, that specifically binds to A3 domain of von Willebrand factor or a fragment thereof.
13C. Any of recitations 11C to 12C, wherein the monoclonal antibody is a humanized antibody having only the hypervariable regions of non-human animal origin.
14C. Any of recitations 11C to 13C, wherein the monoclonal antibody is a humanized antibody having only the hypervariable regions of rodent origin.
15C. The monoclonal antibody of recitation 11C or 14C for use as a medicament, the monoclonal antibody or an antigen-binding fragment or recombinant binding protein thereof having a reactivity substantially identical to the monoclonal antibody obtained from a cell line that has been deposited with the Belgian Collections of Microorganisms under accession number LMBP 5606CB.
16C. The ligand according to any of recitations 1C to 15C for use as a medicament, wherein the ligand does not induce severe decline of circulating vWF levels or a severe decline in platelet count when administered to a primate by bolus intravenous administration at a dose up to 600 μg/kg.
17C. The ligand according to any of recitations 1C to 16C for use as a medicament, wherein the ligand does not result in severe prolongation of bleeding time or does not induce thrombocytopenia when administered to a primate by bolus intravenous administration at a dose up to 600 μg/kg.
18C. The ligand according to any of recitations 1C to 17C for use as a medicament, wherein the ligand does occupy vWF and inhibits vWF-collagen binding when administered at a therapeutically effective dose up to 600 μg/kg to a primate by bolus intravenous administration.
19C. The ligand according to any of recitations 1C to 18C for use as a medicament, wherein the ligand does not induce severe decline of circulating vWF levels, severe decline in platelet count, severe prolongation of bleeding time or thrombocytopenia and that does not drastically affect clotting time (Prothrombin Time (PT) or activated Partial Thromboplastin Time (aPTT)) and the ligand does inhibit vWF-collagen binding and induces increased vWF-occupancy when administered to a primate by bolus intravenous administration at a therapeutically effective dose up to 600 μg/kg.
20C. Any of recitations 9C to 19C, wherein the ligand is in an immunoconjugate with a thrombolytic agent.
21C. The ligand according to recitation 20C, wherein the immunoconjugate contains a thrombolytic agent or a recombinant variant or fragment thereof, the thrombolytic agent being selected from the group consisting of compounds that neutralize α2-AP or increase fibrinolysis are, for example, plasmin, mini-plasmin (lacking the first four kringles), micro-plasmin (lacking all five kringles) or human plasmin-forming proteins, including lys-plasminogen or similar substances.
22C. The ligand of any of recitations 1C to 19C for use in a medicine that by interfering with the vWF-collagen interaction in an individual, inhibits platelet tethering to a blood vessel surface under high shear stress or at high shear rates.
23C. The ligand of any of recitations 1C to 19C for use in a medicine that by interfering with the vWF-collagen interaction, inhibits the first steps of thrombus formation in an individual.
24C. The ligand of any of recitations 1C to 19C for use in a medicine that by interfering with the vWF-collagen interaction, blocks the first steps of thrombus formation before platelet activation and platelet secretion of vasoactive compounds that induce smooth muscle cell migration and proliferation resulting in restenosis.
25C. The ligand of any of recitations 1C to 19C for use in an anti-thrombotic treatment.
26C. The ligand of any of recitations 1C to 19C for use in an anti-thrombotic treatment to prevent the formation of a non-occlusive thrombus.
27C. The ligand of any of recitations 1C to 19C for use in an anti-thrombotic treatment to prevent the formation of an occlusive thrombus.
28C. The ligand of any of recitations 1C to 19C for use in an anti-thrombotic treatment to prevent arterial thrombus formation.
29C. The ligand of any of recitations 1C to 19C for use in an anti-thrombotic treatment to prevent acute coronary occlusion.
30C. The ligand of any of recitations 1C to 19C for use in an anti-thrombotic treatment to maintain the patency of diseased arteries.
31C. The ligand of any of recitations 1C to 19C for use in an anti-thrombotic treatment to prevent restenosis.
32C. The ligand of any of recitations 1C to 19C for use in an anti-thrombotic treatment to prevent restenosis after PCTA or stenting.
33C. The ligand of any of recitations 1C to 19C for use in an anti-thrombotic treatment to prevent thrombus formation in stenosed arteries.
34C. The ligand of any of recitations 1C to 19C for use in an anti-thrombotic treatment to prevent hyperplasia after angioplasty, atherectomy or arterial stenting.
35C. The ligand of any of recitations 1C to 19C for use in an anti-thrombotic treatment to prevent unstable angina.
36C. The ligand of any of recitations 1C to 19C for use in an anti-thrombotic treatment to prevent or treat the occlusive syndrome in a vascular system.
37C. A pharmaceutical composition comprising the ligand of any of recitations 1C to 36C in admixture with a pharmaceutically acceptable carrier.
38C. The pharmaceutical composition of recitation 37C, further comprising a thrombolytic agent in a form either for simultaneous or sequential use.
39C. Use of the ligand of any of recitations 1C to 25C for the manufacture of a medicament for use in the treatment of a thrombotic disorder in an individual in need thereof.
40C. Use of the ligand of any of recitations 1C to 25C for the manufacture of a medicament for use in an anti-thrombotic treatment of any of recitations 27C to 36C.
41C. Use of the ligand of any of recitations 1C to 25C for the manufacture of a medicament that by interfering in an individual with the vWF-collagen interaction under high shear stress inhibits platelet tethering to a damaged blood vessel surface.
42C. Use of the ligand of any of recitations 1C to 25C for the manufacture of a medicament that by interfering with the vWF-collagen interaction, inhibits the first steps of thrombus formation in an individual.
43C. Use of the ligand of any of recitations 1C to 25C for the manufacture of a medicament that by interfering with the vWF-collagen interaction, blocks the first steps of thrombus formation before platelet activation or before activated platelet secretion of vaso-activating compounds that induce smooth muscle cell migration and cell proliferation resulting in restenosis.
44C. An anti-thrombotic agent that binds with the A3 domain of von Willebrand factor or an epitope thereof, resulting in inhibition of interaction of von Willebrand factor with collagen and that does not induce severe bleeding disorders in an individual at a minimal medicinal effective dose to exhibit anti-thrombotic action.
45C. The anti-thrombotic agent of recitation 44C that does not induce severe decline of circulating vWF levels or a severe decline in platelet count at a minimal medicinal effective dose to exhibit anti-thrombotic action.
46C. The anti-thrombotic agent of recitation 44C that does not result in severe prolongation of bleeding time or does not induce thrombocytopenia at a minimal medicinal effective dose to exhibit anti-thrombotic action.
47C. The anti-thrombotic agent of recitation 44C that does inhibit vWF-collagen binding and increases vWF-occupancy at a minimal medicinal effective dose to exhibit anti-thrombotic action.
48C. The anti-thrombotic agent of recitation 44C that does not induce severe decline of circulating vWF-Ag, severe decline in platelet count, severe prolongation of bleeding time or thrombocytopenia and that does not drastically affect clotting time (Prothrombin Time (PT) or activated Partial Thromboplastin Time (aPTT)) and that does inhibit vWF-collagen binding and induces increased vWF-occupancy at a minimal medicinal effective dose to exhibit anti-thrombotic action.
49C. The anti-thrombotic agent of any of recitations 44C to 48C, wherein the anti-thrombotic is an antibody, monoclonal antibody or a fragment Fab, Fab′ or F(ab′)2 thereof or a homologue of the fragment.
50C. A method of anti-thrombotic therapy in an individual, comprising administering to the individual at risk of thrombosis, a therapeutically effective amount of the anti-thrombotic agent that inhibits the binding of von Willebrand factor to collagen of a damaged blood vessel wall.
51C. The method of anti-thrombotic therapy of recitation 50C, wherein the effective amount of anti-thrombotic agent inhibits platelet tethering to a damaged blood vessel surface.
52C. A method for screening and selecting a medicinally effective and acceptable anti-thrombotic agent that inhibits von Willebrand collagen binding comprising: a) characterizing agents that inhibit von Willebrand collagen binding; b) administering the agent to a mammal and preferably to a primate with injured blood vessel; c) selecting the agents that at a dose that significantly reduces cyclic flow reductions (CFR), do not drastically affect platelet count, do not drastically increase bleeding time, do not drastically change clotting time as measured by an assay such as activated Partial Thromboplastin Time or Prothrombin Time and do not drastically affect circulating vWF levels.
53C. A polynucleotide encoding for the antigen binding Fab, Fab′ or F(ab′)2 fragment of recitation 12C.
54C. A DNA probe for detecting the polynucleotide sequence of recitation 53C, comprising a nucleic acid molecule having a sequence complementary to the coding sequence of the polynucleotide.

Further scope of applicability of the invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention as claimed.

### DESCRIPTION OF THE DRAWINGS

FIG. 1 shows the inhibiting effect of 6B4 Fab fragments on the ristocetin- and botrocetin-induced binding of vWF to rGPIb.

FIG. 2 shows the inhibiting effect of 6B4 Fab fragments on platelet adhesion to collagen type I under flow.

FIG. 3 shows binding curves of 6B4 and its fragments to baboon platelets in plasma.

FIG. 4 shows the inhibitory effect of 6B4 and its fragments on ristocetin-induced baboon platelet aggregation.

FIG. 5 shows platelet adhesion and deposition onto three thrombogenic devices placed in baboons either untreated (FIG. 5, Panel A) or treated (FIG. 5, Panel B) with 6B4 Fab fragments.

FIG. 6 shows the influence of late treatment of baboons with 6B4 Fab fragments on platelet deposition.

FIG. 7 shows the effect of 6B4 Fab fragments on cyclic flow reductions.

FIG. 8 shows the effect of 6B4 Fab fragments on platelet count.

FIG. 9 shows the effect of 6B4 Fab fragments on bleeding time.

FIG. 10 shows the inhibition of ex vivo platelet aggregation by 6B4 Fab fragments.

FIG. 11 shows the occupancy of GPIb receptors by 6B4 Fab fragments.

FIG. 12 shows (lower lines) the amino acid sequence (SEQ ID NO:3) and (upper lines) the nucleotide sequence (SEQ ID NO: 1) for the variable regions VL of the light chains of the 6B4 monoclonal antibody.

FIG. 13 shows (lower lines) the amino acid sequence (SEQ ID NO:4) and (upper lines) the nucleotide sequence (SEQ ID NO:2) for the variable regions VH of the heavy chains of the 6B4 monoclonal antibody.

FIG. 14 shows the infarct volumes and functional outcomes 24 hours after focal cerebral ischemia in mice treated with different anti-platelet antibodies. Panel A (top), representative TTC stains of three corresponding coronal brain sections of mice treated with rat IgG (controls), rat IgG Fab (controls), anti-GPIbα Fab (one hour before or one hour after MCAO), and anti-GPVI mAbs. Panel A (bottom), brain infarct volumes in mice treated with control rat IgG (n=10), control rat IgG Fab (n=10), anti-GPIbα Fab (n=12 at one hour before or n=10 at one hour after MCAO), and anti-GPVI mAbs (n=16). Panel B, Neurological Bederson score (left) and grip test (right) as assessed at day 1 after tMCAO for controls (rat IgG, n=10; rat IgG Fab, n=10), mice treated with anti-GPIbα Fab (n=12 at one hour before or n=10 at one hour after MCAO, respectively) and anti-GPVI mAbs (n=16). *P<0.05, **P<0.01, ***P<0.0001, Bonferroni-corrected one-way ANOVA vs. controls.

FIG. 15 displays the MRIs of cerebral infarcts. Serial coronal T2-weighted MR brain images show hyperintense ischemic lesions (white arrows) at days 1 (A, B) and 7 (C) after tMCAO in controls (rat IgG) (A) and anti-GPIbα Fab-treated rats (B, C). Infarcts are smaller in anti-GPIbα Fab-treated mice (B) vs. controls (A), and T2 hyperintensity further decreases at day 7 as a result of a “fogging” effect during infarct maturation (C). Importantly, hypointense areas indicative of ICH were always absent, demonstrating that the selective blockade of the GPIb receptor in platelets does not increase the risk of secondary hemorrhagic transformation even at more advanced stages of infarct development.

FIG. 16 shows the frequency of ICH and mortality rate after tMCAO in mice treated with different doses of anti-GPIIb/IIIa F(ab)₂. Panel A, representative images of a whole brain (left) and two corresponding coronal brain sections (right) from a mouse treated with 100 μg anti-GPIIb/IIIa F(ab)₂ (100% receptor blockade), followed by 60 minutes of tMCAO. Note the massive hemorrhagic transformation (black arrows) within the infarcted brain area. Panel B, percentage of ICH and mortality rate at day 1 after tMCAO in controls (rat IgG and rat IgG Fab, n=10) and mice treated with 100 μg (100% receptor blockade, n=7), 20 μg (78.4% receptor blockade, n=8), and 10 μg (67.8% receptor blockade, n=7) anti-GPIIb/IIIa F(ab)₂. Note that the frequency of ICH and mortality after GPIIb/IIIa blockade after tMCAO are strictly dose-dependent. *P<0.05, χ² test vs. controls.

FIG. 17 displays the infarct volumes 24 hours after focal cerebral ischemia in mice treated with different doses of anti-GPIIb/IIIa F(ab)₂. Top, representative TTC stains of three corresponding coronal brain sections of mice treated with rat IgG (controls), rat IgG Fab (controls), and 100 μg (100% receptor blockade), 20 μg (78.4% receptor blockade), and 10 μg (67.8% receptor blockade) anti-GPIIb/IIIa F(ab)₂. Bottom, brain infarct volumes in mice treated with control rat IgG (n=10), control rat IgG Fab (n=10), and 100 Hg (100% receptor blockade, n=3), 20 μg (78.4% receptor blockade, n=7), and 10 μg (67.8% receptor blockade, n=7) anti-GPIIb/IIIa F(ab)₂. Because of increased intracerebral bleeding and mortality in the group receiving 100 μg (100% receptor blockade) anti-GPIIb/IIIa F(ab)₂, only three animals were available for analysis.

FIG. 18 shows the infarct volumes and functional outcomes 24 hours after focal cerebral ischemia in wild-type mice (WT), in KO mice lacking vWF, a major ligand for GPIb, and wild-type mice treated with the anti-glycoprotein Ib Fab: the infarct size in vWF KO and anti-GPIb-treated mice is significantly reduced. Global neurological status scored according to Bederson et al. (J. B. Bederson et al., Stroke, 1986, 17:472-476) and the grip test score representing motor function and coordination, graded according to P. M. Moran et al. (Proc. Natl. Acad. Sci. U.S.A. 1995, 92:5341-5345) also show significant beneficial effects of both the absence of vWF and of inhibition of GPIb.

FIG. 19: Inhibition of CFR by mAb 82D6A3. Representative records of CFRs showing the effect of a bolus injection of 100 μg/kg and 300 μg/kg mAb 82D6A3.

FIG. 20: Inhibition of CFRs by mAb 82D6A3. Different doses of mAb 82D6A3 were administered to baboons and the CFRs were measured for 60 minutes. Data represent the mean SD with n=3 for 0.1 and 0.3 mg/kg mAb 82D6A3 and n=2 for 0.6 mg/kg.

FIG. 21: Relation between the ex vivo vWF binding to collagen and vWF-occupancy. All mean data measured at the different time points in the three different dose studies were used (Tables II and III).

FIG. 22: Correlation between the in vitro measurements of the vWF binding to collagen and vWF-occupancy. The experiment is a representation of two experiments.

FIG. 23: Relation between the ex vivo vWF-occupancy and mAb 82D6A3 plasma levels. All mean data measured at the different time points in the three different dose studies were used (Tables II and III).

FIG. 24: Inhibition of vWF binding to human collagen type I inhibition of vWF (final concentration 0.5 μg/ml) binding to human collagen type I (▪), type III (), or to calf skin collagen (▴) by 82D6A3 F(ab). Plates were coated with 25 μg/ml, 100 μl/well collagen. Bound vWF was detected.

FIG. 25: Inhibition of platelet deposition onto a human collagen type I. Panel A: Inhibition of platelet deposition onto a human collagen type I coated surface in flow at a shear rate 2600 s⁻¹. Filled bar: no antibody; open bar: 3 μg/ml 82D6A3; hatched bars: different concentrations of 82D6A3 F(ab)-fragments. Panel B: Shear-dependent inhibition of platelet deposition onto a human collagen type I coated surface by 82D6A3. Filled bars: no antibody; open bars: 5 μg/ml 82D6A3 F(ab)-fragments.

FIG. 26: Panel A: Binding of phage clones L15G8 () and L15C5 (▪) to microtiter plates coated with 10 μg/ml 82D6A3. Panel B: Inhibition of the binding of phages L15G8 () and L15C5 (▪) to microtiter plates coated with 10 μg/ml 82D6A3 by vWF. Final concentration LISG8: 2×10⁹/ml; L15C5: 8×10⁹/ml. Bound phages were detected.

FIG. 27: Panel A: Binding of phage clones C6H5 (), C6G12 (▪) and C6A12 (▴) to microtiter plates coated with 10 μg/ml 82D6A3. Panel B: Inhibition of the binding of phages C6H5 (), C6G12 (▪) and C6A12 (▴) to microtiter plates coated with 10 μg/ml MoAb 82D6A3 by vWF. Final concentration of phages: 5×10¹⁰/ml. Bound phages were detected.

FIG. 28: Inhibition of the binding of biotinylated C6H5-phages to microtiter plates coated with 10 μg/ml 82D6A3 by L15G8 phages. C6H5-phages were used at a final concentration of 2×10¹⁰/ml. Bound biotinylated C6H5-phages were detected with streptavidin-HRP.

FIG. 29: Alignment of the vWF sequence with the phage sequences (: similarity, I identity).

### LEGEND TO TABLES

Table I: Platelet counts, plasma levels of 6B4 Fab-fragments, ex vivo ristocetin-induced platelet agglutination and bleeding times following administration of 80 to 640 μg/kg 6B4 Fab fragments to baboons. Values are given as mean±SE. Statistical comparisons were made using student t-test for paired sample groups (p<0.05).

Table II: Effects of Selective Anti-Platelet Antibodies on Hemostasis in Mice.

Table III: Platelet count and bleeding time measured after administration of different doses of mAb 82D6A3 in baboons. Values are mean data+SD, /: not determined.

Table IV: Ex vivo mAb 82D6A3 plasma concentration, vWF-Ag levels, vWF-occupancy and vWF-collagen binding activity measured after administration of 100 and 300 g/kg mAb 82D6A3 to baboons. Data are mean data+SD, of n=9, i.e., at each time point, the plasma samples were measured three times in three different ELISAs and this for the three animal experiments.

Table V: Ex vivo mAb 82D6A3 plasma concentration, vWF-Ag levels, vWF-occupancy and vWF-collagen binding activity measured after administration of 600 g/kg mAb 82D6A3. Data are mean data+SD, of n=6, i.e., at each time point, the plasma samples were three times measured in three different ELISAs and this for the two animal experiments.

### DETAILED DESCRIPTION OF THE INVENTION

It will be apparent to those skilled in the art that various modifications and variations can be made in the treatment of occlusive syndromes in the cerebral vascular system causing transient cerebral infarct due to stroke or ischemic stroke using the antibody or antibody-derivable ligands of the invention and in construction of the system and method without departing from the scope or spirit of the invention. Examples of such modifications have been previously provided.

### DEFINITIONS AND EXPLANATIONS

“Treatment” is herein defined as the application or administration of a bioactive agent such as an antibody or antigen-binding fragment thereof to a mammalian subject or a patient where the patient has a disease, a symptom of a disease, or a predisposition toward a disease, where the purpose is to cure, heal, alleviate, relieve, alter, remedy, ameliorate, improve, or affect the disease, the symptoms of the disease, or the predisposition toward the disease. By “treatment” is also intended the application or administration of a pharmaceutical composition comprising the bioactive agent such as an antibody or fragments thereof to a mammalian subject or a patient who has a disease, a symptom of a disease, or a predisposition toward a disease, where the purpose is to cure, heal, alleviate, relieve, alter, remedy, ameliorate, improve, or affect the disease, the symptoms of the disease, or the predisposition toward the disease.

“Platelet receptor activator” concerns any natural ligand in a subject of a platelet receptor.

The term “antibody” refers to intact molecules, as well as fragments thereof, that are capable of binding to the epitope determinant of the relevant factor or domain of the factor.

“Humanized antibody” as used herein, refers to antibody molecules in which amino acids have been replaced in the non-antigen binding regions in order to more closely resemble a human antibody.

The term “homolog” as used herein with reference to ligands in accordance with the invention refers to a molecule that will compete with or inhibit binding of one of the ligands in accordance with the invention to the target site. The binding should be specific, i.e., the binding of the alternative molecule should be as specific to the site as the ligand in accordance with the invention. Where the ligands in accordance with the invention include amino acid sequences, homology may include having at least about 60%, preferably at least 80%, more preferably at least 90%, and most preferably at least 95% amino acid sequence identity with the relevant ligand.

“Transient cerebral attack” as used herein is a temporary deficiency of blood flow in the brain, more particularly the cerebrum, which is called stroke, if without treatment would last for more than 24 hours and can lead to focal cerebral ischemic infarction (ischemic stroke).

The term “homologue” as used herein with reference to growth factors of the invention refers to molecules having at least 50%, more preferably at least 70%, and most preferably at least 90% amino acid sequence identity with the relevant protein.

The term “fragment” as used herein with reference to the antibodies of the invention refers to molecules that contain the active portion of the antibody, or another bioactive protein, i.e., the portion that is functionally capable of improving perfusion or reducing or suppressing infarct expansion or otherwise enhancing revascularization of cerebral infarcts, and which may have lost a number of non-essential properties of the parent protein, which for parent antibodies is non-essential properties with respect to binding and selectivity. Preferably, the antibody fragment used in the invention contains a binding domain of the relevant antibody.

The term “derivative” as used herein with reference to bioactive proteins of the invention refers to molecules that contain at least the active portion of the parent protein (as defined hereinabove) and a complementary portion that differs from that present in the parent antibody, e.g., by further manipulations such as introducing mutations.

The term “vascular endothelial growth factor” as used herein, refers, whether of human or animal origin, to all isoforms thereof. The term “placenta growth factor” as used herein, refers, whether of human or animal origin, to all isoforms thereof. Growth factor-mediated improved perfusion of the penumbra in the brain or of the jeopardized myocardium of patients suffering ischemic events, either via increased vasodilatation or angiogenesis (the formation of endothelial-lined vessels), may be of great therapeutic value according to Isner et al. in J. Clin. Invest. (1999), 103(9):1231-6, but many questions yet remain to be answered in this respect, e.g., which suitable growth factor or combination of growth factors should be selected and which route of administration is effective yet safe for this purpose.

In addition, an outstanding question is whether formation of new endothelial-lined vessels (i.e., angiogenesis) alone is sufficient to stimulate sustainable functional tissue perfusion. Indeed, coverage of endothelial-lined vessels by vascular smooth muscle cells (i.e., arteriogenesis) provides vasomotor control, structural strength and integrity and renders new vessels resistant to regression. Capillary blood vessels consist of endothelial cells and pericytes, which carry all the genetic information required to form tubes, branches and entire capillary networks. Specific angiogenic molecules can initiate this process. A number of polypeptides that stimulate angiogenesis have been purified and characterized as to their molecular, biochemical and biological properties, as reviewed by Klagsbrun et al. in Ann. Rev. Physiol. (1991) 53:217-239, and by Folkman et al. in J. Biol. Chem. (1992) 267:10931-4. One factor that can stimulate angiogenesis and that is highly specific as a mitogen for vascular endothelial cells is termed “vascular endothelial growth factor” (hereinafter referred to as “VEGF”) according to Ferrara et al. in J. Cell. Biochem. (1991) 47:211-218. VEGF is also known as vasculotropin. Connolly et al. also describe in J. Biol. Chem. (1989) 264:20017-20024, in J. Clin. Invest. (1989) 84:1470-8, and in J. Cell. Biochem. (1991) 47:219-223, a human vascular permeability factor that stimulates vascular endothelial cells to divide in vitro and promotes the growth of new blood vessels when administered into healing rabbit bone grafts or rat corneas.

The term “vascular permeability factor” (“VPF” for abbreviation) was adopted because of increased fluid leakage from blood vessels following intradermal injection and appears to designate the same substance as VEGF. The murine VEGF gene has been characterized and its expression pattern in embryogenesis has been analyzed. A persistent expression of VEGF was observed in epithelial cells adjacent to fenestrated endothelium, e.g., in choroid plexus and kidney glomeruli, which is consistent with its role as a multifunctional regulator of endothelial cell growth and differentiation as disclosed by Breier et al. in Development (1992) 114:521-532. VEGF shares about 22% sequence identity, including a complete conservation of eight cysteine residues, according to Leung et al. in Science (1989) 246:1306-9, with human platelet-derived growth factor PDGF, a major growth factor for connective tissue.

Alternatively spliced mRNAs have been identified for both VEGF and PDGF and these splicing products differ in their biological activity and receptor-binding specificity. VEGF is a potent vasoactive protein that has been detected in and purified from media conditioned by a number of cell lines including pituitary cells, such as bovine pituitary follicular cells (as disclosed by Ferrara et al. in Biochem. Biophys. Res. Comm. (1989) 161:851-858, and by Gospodarowicz et al. in Proc. Natl. Acad. Sci. USA (1989) 86:7311-5), rat glioma cells (as disclosed by Conn. et al. in Proc. Natl. Acad. Sci. USA (1990) 87:1323-1327), and several tumor cell lines. Similarly, an endothelial growth factor isolated from mouse neuroblastoma cell line NB41 with an unreduced molecular mass of 43-51 kDa has been described by Levy et al. in Growth Factors (1989) 2:9-19.

VEGF was characterized as a glycosylated cationic 46 kDa dimer made up of two sub-units each with an apparent molecular mass of 23 kDa. It is inactivated by sulfhydryl-reducing agents, resistant to acidic pH and to heating, and binds to immobilized heparin. VEGF has four different forms of 121, 165, 189 and 206 amino acids due to alternative splicing of mRNA. The various VEGF species are encoded by the same gene. Analysis of genomic clones in the area of putative mRNA splicing also shows an intron/exon structure consistent with alternative splicing. The VEGF165 species is the molecular form predominantly found in normal cells and tissues. The VEGF 121 and VEGF165 species are soluble proteins and are capable of promoting angiogenesis, whereas the VEGF 189 and VEGF206 species are mostly cell-associated. All VEGF isoforms are biologically active, e.g., each of the species when applied intradermally is able to induce extravasation of Evans blue. However, VEGF isoforms have different biochemical properties that may possibly modulate the signaling properties of the growth factors. The VEGF 165, VEGF 189 and VEGF206 species contain eight additional cysteine residues within the carboxy-terminal region. The amino-terminal sequence of VEGF is preceded by 26 amino acids corresponding to a typical signal sequence. The mature protein is generated directly following signal sequence cleavage without any intervening prosequence. Other VEGF polypeptides from the PDGF family of growth factors have been disclosed in U.S. Pat. No. 5,840,693. Purified and isolated VEGF-C cysteine deletion variants that bind to a VEGF tyrosine kinase receptor have been disclosed in U.S. Pat. No. 6,130,071. VEGF and PIGF can also form heterodimers and have been documented in vivo (Y. Cao, P. Linden, D. Shima, F. Browne and J. Folkman, “In vivo angiogenic activity and hypoxia induction of heterodimers of placenta growth factor/vascular endothelial growth factor,” J. Clin. Invest. 98, 2507-11, 1996; J. DiSalvo et al., “Purification and characterization of a naturally occurring vascular endothelial growth factor placenta growth factor heterodimer,” J. Biol. Chem. 270, 7717-23, 1995). Their role in angiogenesis and arteriogenesis in vivo remains controversial, and no information is available whether VEGF/PIGF heterodimers can be used for therapeutic applications. VEGF/PIGF heterodimers are obtainable from R&D, Abbingdon, UK.

Like other cytokines, VEGF can have diverse effects that depend on the specific biological context in which it is found. The expression of VEGF is high in vascularized tissues (e.g., lung, heart, placenta and solid tumors) and correlates with angiogenesis both temporally and spatially. VEGF has been shown to directly contribute to induction of angiogenesis in vivo by promoting endothelial cell growth during normal embryonic development, wound healing, tissue regeneration, and reorganization. Therefore, VEGF has been proposed for use in promoting vascular tissue repair, as disclosed by EP-A-0,506,477. VEGF is also involved in pathological processes such as growth and metastasis of solid tumors and ischemia-induced retinal disorders, such as disclosed in U.S. Pat. No. 6,114,320. VEGF expression is triggered by hypoxia so that endothelial cell proliferation and angiogenesis appear to be especially stimulated in ischemic areas. Finally, U.S. Pat. No. 6,040,157 discloses human VEGF2 polypeptides that have been putatively identified as novel vascular endothelial growth factors based on their amino acid sequence homology to human VEGF. The latter document further discloses restoration of certain parameters in the ischemic limb by using a VEGF2 protein. However, it is also known by Hariawala et al. in J. Surg. Res. (1996), 63(1):77-82, that a systemic administration of VEGF, in high doses over short periods of time, improves myocardial blood flow but produces hypotension in porcine hearts.

Placenta growth factor (hereinafter referred as “PIGF”) was disclosed by Maglione et al. in Proc. Natl. Acad. Sci. USA (1991), 88(20):9267-71, as a protein related to the vascular permeability factor. U.S. Pat. No. 5,919,899 discloses nucleotide sequences coding for a protein, namely PIGF, which can be used in the treatment of inflammatory diseases and in the treatment of wounds or tissues after surgical operations, transplantations, burns, or ulcers and so on. Soluble non-heparin-binding and heparin-binding forms, built up of 131 and 152 amino acids, respectively, have been described for PIGF, which is expressed in placenta, trophoblastic tumors and cultured human endothelial cells, according to U.S. Pat. No. 5,776,755.

Heavy chain antibodies and methods for obtaining the same have been described in the art; see, for example, the following references that are cited as general background art: WO 94/04678 (EP 656 946), WO 96/34103 (EP 0 822 985) and WO 97/49805 by Vrije Universiteit Brussel; WO 97/49805 by Vlaams Interuniversitair Instituut voor Biotechnologie; WO 94/2559 1 (EP 0 698 097) and WO 00/43507 by Unilever N.V.; WO 01/90190 by the National Research Council of Canada; WO 03/025020 (EP 1 433 793) by the Institute of Antibodies; WO 04/062551, WO 04/041863, WO 04/041865, WO 04/041862 by applicant; as well as, for example, Hamers-Casterman et al., Nature, Vol. 363, p. 446 (1993), and Rieclmiann and Muyldermans, Journal of Immunological Methods, 231 (1999), p. 25-38. For example, heavy chain antibodies against a desired antigen can be obtained from a species of Camelid immunized with the antigen, as described in the general prior art mentioned above.

In particular, a part or a fragment may be a variable domain, such as a heavy chain variable domain and/or a light chain variable domain, or a ScFv comprising both a heavy chain variable domain and/or a light chain variable domain. Such antibodies and fragments, and methods for obtaining the same, will be clear to the skilled person; reference is, for example, made to Roitt et al., Immunology (6th. Ed.), Mosby/Elsevier, Edinburgh (2001); and Janeway et al., Immunobiology (6th Ed.), Garland Science Publishing/Churchill Livingstone, New York (2005).

Stroke, defined as a sudden weakening or loss of consciousness, sensation and voluntary motion caused by rupture or obstruction of an artery of the brain, is the third cause of death in the United States. Worldwide, stroke is the number one cause of death due to its particularly high incidence in Asia. Ischemic stroke is the most common form of stroke, being responsible for about 85% of all strokes, whereas hemorrhagic strokes (e.g., intraparenchymal or subarachnoid) account for the remaining 15%. Due to the increasing mean age of the population, the number of strokes is continuously increasing. Because the brain is highly vulnerable to even brief ischemia and recovers poorly, primary prevention in ischemic stroke prevention offers the greatest potential for reducing the incidence of this disease.

Focal ischemic cerebral infarction occurs when the arterial blood flow to a specific region of the brain is reduced below a critical level. Cerebral artery occlusion produces a central acute infarct and surrounding regions of incomplete ischemia (sometimes referred to as “penumbra”) that are dysfunctional, yet potentially salvageable. Ischemia of the myocardium, as a result of reduced perfusion due to chronic narrowing of blood vessels, may lead to fatal heart failure and constitutes a major health threat. Acute myocardial infarction, triggered by coronary artery occlusion, produces cell necrosis over a time period of several hours. In the absence of reflow or sufficient perfusion, the cerebral or myocardial ischemic regions undergo progressive metabolic deterioration, culminating in infarction, whereas restoration of perfusion in the penumbra of the brain infarct or in the jeopardized but salvageable region of the myocardium may ameliorate the tissue damage.

Also disclosed is a ligand derived from, e.g., a Fab fragment of a monoclonal antibody obtainable from the cell line, deposited with the Belgian Coordinated Collections of Microorganisms under accession number LMBP 5108CB, that binds to the human platelet glycoprotein GPIb and prevents the binding of von Willebrand factor to GPIb without inducing thrombocytopenia, which has been for the first time studied for its for a treatment of hemostasis disorder such as thrombosis and transient cerebral attack and described in WO2001010911 (2000 Aug. 8), GB199918788 1999 08 10, EP2000102032A (2000 Feb. 2) and WO2000EP7874 (2000 Aug. 8), “Cell line ligands and antibody fragments for use in pharmaceutical compositions for preventing and treating hemostasis disorder” as provided hereunder.

The ligand is useful in admixture with a pharmaceutically acceptable carrier, in a pharmaceutical composition, optionally further comprising a thrombolytic agent, for preventing and/or treating hemostasis disorders.

The invention relates to novel cell lines and to ligands, namely human and/or humanized monoclonal antibodies, as well as fragments such as Fab or single variable domains and derivatives and combinations thereof, obtainable from the cell line. It also relates to pharmaceutical compositions comprising the ligands or antibody fragments and to methods of preventing and treating hemostasis disorders, in particular, anti-thrombotic treatments in humans, by administration of the ligands or antibody fragments to patients in need thereof. It further relates to a polynucleotide encoding for the antigen-binding Fab fragment of a monoclonal antibody derivable from the cell line.

The coagulation of blood involves a cascading series of reactions leading to the formation of fibrin. The coagulation cascade consists of two overlapping pathways required for hemostasis. The intrinsic pathway comprises protein factors present in circulating blood, while the extrinsic pathway requires tissue factor that is expressed on the cell surface of a variety of tissues in response to vascular injury. Agents interfering with the coagulation cascade, such as heparin and coumarin derivatives, have well-known therapeutic uses in the prophylaxis of venous thrombosis.

Aspirin also provides a protective effect against thrombosis. It induces a long-lasting functional defect in platelets, detectable clinically as a prolongation of the bleeding time, through inhibition of the cyclooxygenase activity of the human platelet enzyme prostaglandin H-synthase (PGHS-1) with doses as low as 30 to 75 mg. Since gastrointestinal side effects of aspirin appear to be dose-dependent, and for secondary prevention, treatment with aspirin is recommended for an indefinite period, there are practical reasons to choose the lowest effective dose. Further, it has been speculated that a low dose (30 mg daily) might be more anti-thrombotic, but attempts to identify the optimal dosage have yielded conflicting results. It has been claimed that the dose of aspirin needed to suppress full platelet aggregation may be higher in patients with cerebrovascular disease than in healthy subjects and may vary from time to time in the same patient. However, aspirin in any daily dose of 30 mg or higher reduces the risk of major vascular events by 20% at most, which leaves much room for improvement. Further, the inhibiting role of aspirin may lead to prevention of thrombosis and to excess bleeding. The balance between the two depends critically on the absolute thrombotic versus hemorrhagic risk of the patient.

In patients with acute myocardial infarction, reduction of infarct size, preservation of ventricular function and reduction in mortality has been demonstrated with various thrombolytic agents. However, these agents still have significant shortcomings, including the need for large therapeutic doses, limited fibrin specificity, and significant associated bleeding tendency.

Recombinant tissue plasminogen activator (t-PA) restores complete patency in just over one-half of patients, whereas, streptokinase achieves this goal in less than one third. Further, reocclusion after thrombolytic therapy occurs in 5 to 10% of cases during the hospital stay and in up to 30% within the first year according to Verheugt et al., J. Am. Coll. Cardiol. (1996) 27:618-627. Numerous studies have examined the effects of adjunctive anti-thrombin therapy for patients with acute myocardial infarction. For instance, U.S. Pat. No. 5,589,173 discloses a method for dissolving and preventing reformation of an occluding thrombus comprising administering a tissue factor protein antagonist, such as a monoclonal or polyclonal antibody, in adjunction to a thrombolytic agent.

In arterial blood flow, the platelet adhesion is mainly supported by the platelet receptor glycoprotein (GP) Ib which interacts with von Willebrand factor (vWF) at the site of vessel wall injury. Blood platelets, through the processes of adhesion, activation, shape change, release reaction and aggregation, form the first line of defense when blood vessels are damaged. They form a hemostatic plug at the site of injury to prevent excessive blood loss. Extensive platelet activation, however, may overcome the normal thrombo-regulatory mechanisms that limit the size of the hemostatic plug. Platelets then become major prothrombotic offenders predisposing to vaso-occlusive disease.

The formation of a platelet plug during primary hemostasis and of an occluding thrombus in arterial thrombosis involves common pathways. The first event is platelet adhesion to subendothelial collagen, exposed upon vessel injury, which can be a ruptured atherosclerotic plaque. Circulating vWF binds to the collagen and, under the influence of high shear stress, undergoes a conformational change allowing it to bind to its receptor, GPIb/IX/V, on the platelet membrane. This interaction is essential in order to produce a thrombus, at least in smaller vessels or stenosed arteries where shear stress is high, and results in slowing down the progress of the platelets across the damaged surface. Full immobilization of platelets occurs when collagen binds to its receptor GPIa/IIa (integrin α2β1). In addition, collagen activates platelets mainly by binding to GPVI, another collagen receptor. When platelets are activated, GPIIb/IIIa (integrin αIIβ3) undergoes a conformational change and acquires the ability to bind to fibrinogen and vWF, which cross-link adjacent platelets to finally form platelet aggregates.

Lately much effort has been directed to develop antibodies and peptides that can block the binding of the adhesive proteins to GPIIb/IIIa and many of these are being tested in clinical trials. One approach to blocking platelet aggregation involves monoclonal antibodies specific for GPIIb/IIIa receptors.

Specifically, a murine monoclonal antibody named 7E3 useful in the treatment of human thrombotic diseases is described in EP-A-206,532 and U.S. Pat. No. 5,387,413. However, it is known in the art that murine antibodies have characteristics that may severely limit their use in human therapy. As foreign proteins, they may elicit an anti-immunoglobulin response termed human anti-mouse antibody (HAMA) that reduces or destroys their therapeutic efficacy and/or provokes allergic or hypersensitivity reactions in patients, as taught by Jaffers et al., Transplantation (1986) 41:572. The need for readministration in therapies of thromboembolic disorders increases the likelihood of such immune reactions. While the use of human monoclonal antibodies would address this limitation, it has proven difficult to generate large amounts of such antibodies by conventional hybridoma technology.

Recombinant technology has, therefore, been used to construct “humanized” antibodies that maintain the high binding affinity of murine monoclonal antibodies but exhibit reduced immunogenicity in humans. In particular, there have been suggested chimeric antibodies in which the variable region (V) of a non-human antibody is combined with the constant (C) region of a human antibody. As an example, the murine Fc fragment was removed from 7E3 and replaced by the human constant immunoglobulin G region to form a chimera known as c7E3 Fab or abciximab. Obtaining of such chimeric immunoglobulins is described in detail in U.S. Pat. No. 5,770,198.

The potential for synergism between GPIIb/IIIa inhibition by monoclonal antibody 7E3 Fab and thrombolytic therapy was evaluated by Kleiman et al., J. Am. Coll. Cardiol. (1993) 22:381-389. Major bleeding was frequent in this study. Hence, the potential for life-threatening bleeding is clearly a major concern with this combination of powerful anti-thrombotic compounds.

The GPIb-vWF axis, therefore, presents an attractive alternative to GPIIb/IIIa-fibrinogen as a target for platelet inhibition, since a suitable inhibitor might be expected to down-regulate other manifestations of platelet activity, such as granule release, thought to play a role in the development of arteriosclerosis. Activation of platelets is accompanied by secretion of vasoactive substances (thromboxane A2, serotonin) as well as growth factors such as PGDF. Therefore, early inhibition of platelet activation and, hence, prevention of the secretion of their growth and migration factors, via a GPIb blocker, would reduce the proliferation of smooth muscle cells and restenosis after thrombolytic therapy. Moreover, the interaction of GPIb with the damaged vessel wall (adhesion, as well as aggregation and secretion of platelet content) is highly blood flow dependent. Unlike GPIIb/IIIa interactions, GPIb-vWF interaction occurs exclusively under the high flow conditions, as occurs in small arteries or created by arterial stenoses. Hence, GPIb inhibition represents theoretically an ideal way to target effective platelet inhibition to damaged arterial areas. GPIb inhibition, therefore, appears particularly suited to treat patients with acute coronary syndromes, transient cerebral attacks and claudication due to peripheral arterial diseases, including prevention of the frequently lethal thrombotic complications of acute coronary syndromes, angioplasty, unstable angina and myocardial infarction.

Despite these potential advantages, the development of compounds that interfere with the vWF-GPIb axis has lagged behind. Only a few in vivo studies described the effects of inhibition of platelet adhesion on thrombogenesis. They include the use of anti-vWF monoclonal antibodies, GPIb binding snake venom proteins like echicetin and crotalin, aurin tricarboxylic acid that binds to vWF and recombinant vWF fragments like VCL, all of which inhibit vWF-GPIb interaction. All these molecules were anti-thrombotic, particularly in studies where a thrombus was formed under high shear conditions. U.S. Pat. No. 5,486,361 discloses a monoclonal antibody 4H12 that specifically binds to the a chain of GPIb and, by means of this interaction, totally inhibits the binding of thrombin to normal human platelets. In addition, it inhibits more than 90% of thrombin-induced von Willebrand factor or fibrinogen binding to platelets. Further, 4H12 does not inhibit ristocetin- or botrocetin-induced binding of von Willebrand factor to platelets, which indicates that this antibody does not prevent von Willebrand factor binding to GPIb.

A number of potent inhibitory anti-GPIb antibodies, such as LJIb1 disclosed by F. Pareti et al. in British Journal of Haematology (1992) 82:81-86, have been produced and were extensively tested with respect to their in vitro effect under both static (platelet agglutination, vWF-binding) and flow conditions. However, for none of these anti-human GPIb antibodies, an in vivo anti-thrombotic effect could be demonstrated. In vivo data obtained by B. Becker and J. L. Miller (Blood (1989) 2:680-694) describe the effect of injecting guinea pigs with intact antibody or F(ab′)2 fragments of PG1, a monoclonal anti-guinea pig GPIb antibody. After intraperitoneal injection of the intact antibody, a hemorrhagic state was produced with a significant lengthening of the bleeding time and drop of the platelet count to 50% of its baseline value. Injection of 0.63 to 2.5 mg/kg of the F(ab′)2 fragments did not decrease the platelet count more than 21%, and bleeding times never increased by more than one minute over baseline values. However, in this particular study the anti-thrombotic effect of the F(ab′)2 fragments was not further investigated by, e.g., testing the fragments in an animal thrombosis model.

In a follow-up study, J. L. Miller et al., Arterioscler. Thromb. (1991), 11:1231-6, disclosed that the F(ab′)2 fragments of PG1 in guinea pigs could effectively reduce thrombus formation on a laser-induced injury.

Unfortunately, this antibody does not cross-react with human platelets and, therefore, it has no further clinical relevance for human therapy.

Part of this rather surprising lack of in vivo studies is due to the low cross-reactivity of the anti-human GPIb monoclonal antibodies with platelets from commonly used laboratory animals. This predisposes to the use of non-human primates as experimental animals. However, even then, attempts to perform in vivo studies are hampered because injection of the anti-GPIb monoclonal antibodies, as well as the snake venom protein echicetin that reacts with GPIb, invariably causes severe thrombocytopenia.

One persistent concern with all available thrombolytic and anti-thrombotic agents, including aspirin, is to induce a risk of overdose and, therefore, of excessive and life-threatening bleeding. Therefore, a first goal of the invention is to provide a thrombus formation protective means by providing a platelet adhesion inhibitor that does not induce a risk of bleeding.

A second goal of the invention is to provide a thrombus formation protective means by providing an inhibitor of platelet adhesion without incurring the risk of thrombocytopenia. A third goal of the invention is the targeting of platelet adhesion, activation and aggregation under high shear conditions, which is of particular importance in the setting of highly stenotic atherosclerotic lesions. The specific targeting of highly stenotic areas in the circulation should make GPIb inhibition particularly suitable for treating unstable angina and in the chronic prevention of arterial occlusion. Unlike with GPIIb/IIIa inhibition, platelet aggregation as well as hemostasis is not expected to be inhibited in low shear vessels, i.e., in veins and normal arteries. Bleeding complications from these vessels by inhibition of GPIb may, therefore, be expected to be better reduced than with GPIIb/IIIa inhibition.

The essence of this invention is that by using a ligand such as a monovalent Fab fragment of a certain inhibitory human GPIb antibody, a marked prevention of platelet-dependent thrombus formation targeted to high shear flow vessels and without incurring thrombocytopenia can be obtained.

Moreover, this is so far the only anti-human GPIb monoclonal antibody for which the anti-thrombotic efficacy has been proven in vivo in an animal thrombosis model.

The invention, therefore, first includes a cell-line deposited with the Belgian Coordinated Collections of Microorganisms under accession number LMBP 5108CB. Secondly, the invention includes a ligand that binds to the human platelet glycoprotein GPIb and prevents the binding of von Willebrand factor (vWF) to GPIb and that preferably does not produce thrombocytopenia when administered to a primate (herein, the word “primate” also relates to humans) at a dose of up to at least 4 mg/kg by bolus intravenous administration. In particular, the invention includes a ligand derived from a monoclonal antibody such as 6B4 obtainable from the cell line.

Third, the invention relates to an antigen-binding Fab fragment, or a homolog or derivative of such fragment (including a humanized fragment that might be divalent, trivalent or tetravalent), which may be obtained by proteolytic digestion of the monoclonal antibody by papain, using methods well known in the art.

Fourth, the invention includes pharmaceutical compositions comprising ligands or fragments that are useful for preventing and treating hemostasis disorders, in particular, for anti-thrombotic treatments in humans.

Finally, the invention includes polynucleotide sequences encoding for the above-mentioned monoclonal antibodies or Fab fragments thereof. It will be appreciated that a multitude of nucleotide sequences fall under the scope of the invention as a result of the redundancy in the genetic code. The invention also includes nucleic acid molecules comprising sequences that are complementary to the coding sequence of the polynucleotides and the use of such molecules as DNA probes for detecting the polynucleotides.

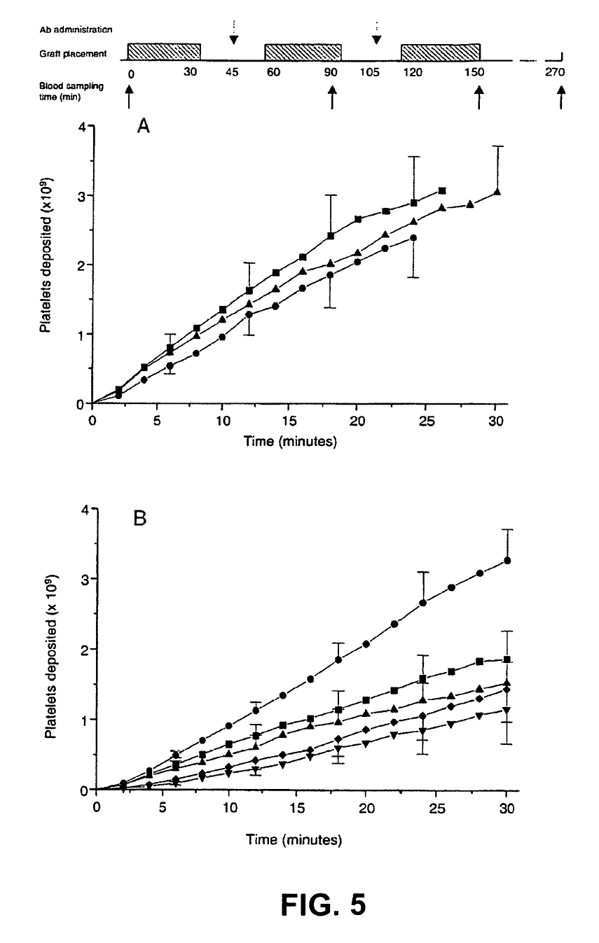

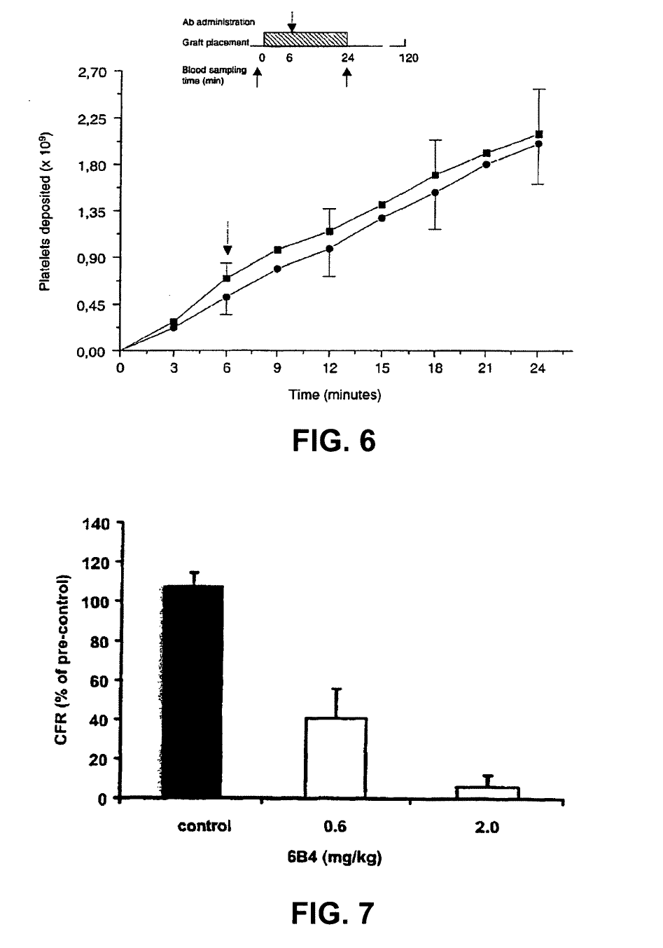

The invention is first based on the observation of the anti-thrombotic effect of human platelet glycoprotein GPIb blocking monoclonal antibody 6B4 Fab fragment derived from the cell line LMBP 5108CB in a baboon model of arterial thrombosis. Two in vivo models were used and described in this invention: the first model is an arteriovenous shunt model in which an extracorporeal loop is made between the femoral artery and the femoral vein. Within this loop, a collagenic graft is incorporated and the platelet deposition onto this graft is measured, as shown in Examples 7 and 8 and FIGS. 5 and 6. Baboons were either pre-treated with the Fab fragment to study the effect on platelet deposition on a thrombogenic device, or treated six minutes after placement of the thrombogenic device in order to investigate the effect on inter-platelet cohesion. In this first study, it was observed that blockade of GPIb had no effect on platelet deposition onto a fresh thrombus, whereas pre-treatment effectively reduced thrombus formation.

The second model is a clinically even more relevant model mimicking platelet-mediated thrombotic occlusion as occurring in stenosed and intimally damaged coronary arteries in vivo. In this second model, a stenosis is applied to a damaged femoral artery, and blood flow is measured. Due to platelet aggregate formation, the stenotic area occludes but reopens due to embolization, resulting in regular cyclic flow reductions as shown in Example 9.

Secondly, the invention is based on in vitro and in vivo studies of the anti-thrombotic efficacy of the monoclonal antibody, 6B4 (IgG1), raised against human platelet glycoprotein lb. In vitro, 6B4 potently inhibits the binding of vWF to human GPIb, both under static and flow conditions, as further illustrated by the following examples, and it also binds to baboon platelets.

When 6B4 was injected into baboons, both the intact monoclonal antibody and its F(ab′)2 fragments caused immediate and severe thrombocytopenia, whereas Fab fragments of 6B4 did not. Furthermore, Fab fragments studied in the two baboon models effectively prevented platelet-dependent arterial thrombosis.

The invention will be described with reference to certain embodiments and figures but the invention is not limited thereto, but only by the following claims.

The invention provides a cell-line deposited with the Belgian Coordinated Collections of Microorganisms under accession number LMBP5108CB. The invention further provides cell lines producing monoclonal antibodies having a reactivity, namely, a reactivity towards human GPIb, substantially identical to that of monoclonal antibodies obtainable or obtained from cell line LMBP 5108CB, as well as the human monoclonal antibodies obtainable from the further cell lines.

The invention also provides ligands that are able to bind to the human platelet glycoprotein GPIb and also preferably able to prevent the binding of von Willebrand factor (vWF) to GPIb, in particular, ligands derived from a monoclonal antibody (referred to as “6B4”) obtainable from the cell line LMBP 5108CB or from equivalent cell lines, such as above defined. More preferably, such a ligand should be able to recognize an epitope located on human platelet glycoprotein GPIb. For instance, the invention relates to ligands of the above-mentioned type, being derived from a monoclonal antibody produced by intentional immunization in animals.

The invention also provides an antigen-binding Fab fragment, or a homolog or derivative of such fragment, which may be obtained by proteolytic digestion of the monoclonal antibody by papain, using methods well known in the art. In order to reduce the immunogenicity of the murine anti-GPIb monoclonal antibody 6B4, the invention also includes the construction of a chimeric antibody, preferentially as a single-chain variable domain that combines the variable region of the mouse antibody with a human antibody constant region, a so-called humanized monoclonal antibody. The monoclonal antibodies produced in animals may be humanized, e.g., by associating the binding complementarity-determining region (“CDR”) from the non-human monoclonal antibody with human framework regions, in particular, the constant C region of human gene, such as disclosed by Jones et al. in Nature (1986) 321:522, or Riechmann in Nature (1988) 332:323, or otherwise hybridized.

This invention also provides using a ligand or a humanized or hybridized monoclonal antibody or an antigen-binding Fab fragment, such as specified hereinbefore, as a medicament. Although aspirin will continue to be widely used for patients with vascular disease, there are, however, a number of situations in which increased thrombotic risk requires the use of a more potent platelet inhibitor than aspirin. Conditions such as angioplasty, coronary stenting and thrombolysis are likely to require more potent platelet inhibitors. In these acute clinical situations, the fibrous cap over an atherosclerotic plaque has been ruptured, which produces deep arterial injury and exposes a much more thrombogenic surface. Furthermore, high shear forces acting on platelets passing through severely narrowed stenoses can also overcome the inhibitory effects of aspirin. Therefore, a GPIb antagonist according to the invention may be used for reducing the problems of occlusion and restenosis in patients undergoing angioplasty or for the prevention of reocclusion after successful thrombolysis by tissue plasminogen activators, streptokinase or the like. It is believed that platelet activation, as a result of the platelet adhesion, is a key component in the failure of thrombolysis. Therefore, a therapeutic approach towards blocking the GPIb-vWF interaction that results in a down-regulation of platelet signaling represents a new way of interfering in thrombus formation.

The invention, therefore, further provides pharmaceutical compositions comprising a ligand or a humanized or hybridized monoclonal antibody or an antigen-binding Fab fragment such as specified hereinbefore, in admixture with a pharmaceutically acceptable carrier. More preferably, the pharmaceutical composition comprises a human or humanized or hybridized monoclonal antibody or an antigen-binding Fab fragment thereof obtainable from the cell line LMBP 5108CB, which are useful for preventing and treating hemostasis disorders, in particular, for anti-thrombotic treatments, in humans.

The use of a GPIb blocker according to the invention is believed to be more efficient in acute situations and, in some cases, as an adjunctive therapy together with other agents such as, among others, aspirin or heparin.

The pharmaceutical composition of the invention may, therefore, further comprise, in view of the so-called adjunctive therapy, a therapeutically effective amount of a thrombolytic agent. Such thrombolytic agents, as well as their usual dosage depending on the class to which they belong, are well known to those skilled in the art. Among numerous examples of thrombolytic agents that may be included in the pharmaceutical compositions of the invention, may be cited tissue plasminogen activators (t-Pa), streptokinase, reptilase, TNK-t-Pa or staphylokinase. The pharmaceutical composition should comprise the additional thrombolytic agent in a form that is suitable, either for simultaneous use or for sequential use. Sequential, as used herein, means that the ligand or humanized monoclonal antibody or antigen-binding Fab fragment of the invention on the one hand and the known thrombolytic agent are administered to the patient in alternance, but not within the same dosage unit.

Suitable pharmaceutical carriers for use in the pharmaceutical compositions of the invention are described, e.g., in Remington's Pharmaceutical Sciences 16th ed. (1980), and their formulation is well known to those skilled in the art. They include any and all solvents, dispersion media, coatings, antibacterial and antifungal agents (for example, phenol, sorbic acid, and chlorobutanol), isotonic agents (such as sugars or sodium chloride) and the like. Additional ingredients may be included in order to control the duration of action of the monoclonal antibody or Fab fragment active ingredient in the composition. Control release compositions may thus be achieved by selecting appropriate polymer carriers, such as, for example, polyesters, polyamino acids, polyvinyl pyrrolidone, ethylene-vinyl acetate copolymers, methylcellulose, carboxymethyl cellulose, protamine sulfate and the like. The rate of drug release and duration of action may also be controlled by incorporating the monoclonal antibody active or Fab fragment ingredient into particles, e.g., microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition comprising the active ingredient may require protective coatings.

The pharmaceutical form suitable for injection includes sterile aqueous solutions or dispersions and sterile powders for the extemporaneous preparation thereof. Typical carriers, therefore, include biocompatible aqueous buffers, ethanol, glycerol, propylene glycol, polyethylene glycol and mixtures thereof.

The pharmaceutical composition and medicament in accordance with the invention may be provided to a patient by means well known in the art, i.e., orally, intranasally, subcutaneously, intramuscularly, intradermally, intravenously, intra-arterially, parenterally or by catheterization. For the reasons stated above, they will be especially useful for the treatment and/or prevention of disorders of hemostasis and particularly for anti-thrombotic treatment or prevention. Therefore, the invention further provides a method of treatment and/or prevention of such disorders by administering to a patient in need thereof a therapeutically effective amount of a ligand or a humanized monoclonal antibody or an antigen-binding Fab fragment such as specified hereinbefore, optionally together with (simultaneously or sequentially) a therapeutically effective amount of a thrombolytic agent such as above described.

The invention also provides a polynucleotide sequence encoding for the antigen-binding Fab fragment, or homolog or derivative of the monoclonal antibody derived from cell line LMBP 5108CB. The invention also provides nucleic acid molecules comprising a sequence that is complementary to the coding sequence of the polynucleotide and the use of such molecules as DNA probes for detecting the polynucleotide.

The invention is further described by the following examples that are provided for illustration purposes only. Data were tested for statistically significant differences. Data given in the text are mean SE. P-values<0.05 are considered significantly different.

Damage of an arterial vessel wall leads to platelet adhesion, aggregation and ultimately may result in thrombosis. These events are known to contribute to the development of occlusive syndromes in the coronary, cerebral and peripheral vascular system, as well as restenosis and intimal hyperplasia that occur after angioplasty, atherectomy and arterial stenting (J. D. Folts, A. I. Schafer, J. Loscalzo, J. T. Willerson, and J. E. Muller, J. Am. Coll. Cardiol. 1999, 33(2):295-303; and A. I. McGhie et al., Circulation 1994, 90(6):2976-2981). In both thrombosis and reocclusion, platelets adhere to the subendothelium of damaged blood vessels through an interaction with von Willebrand factor (vWF) that forms a bridge between collagen, a component of the damaged vessel wall and the platelet glycoprotein Ib (GPIb) (J. J. Sixma, Wester. J. Semin. Hematol. 1977, 14(3):265-299). This reversible adhesion or tethering of the platelets at high shear rate is followed by a firm adhesion through the collagen receptors (GPIa-IIa; GPIV) (B. Kehrel, Semin. Thromb. Hemost. 1995, 21 (2):123-129) resulting in platelet activation and release of ADP, thromboxane, and serotonin. These in turn activate additional platelets and trigger the conformational activation of the platelet GPIIb/IIIa receptor, leading to fibrinogen binding and finally to platelet aggregation (D. R. Phillips, I. F. Charo, R. M. Scarborough et al., Cell 1991, 65(3):359-362). Ultimately, a platelet-initiated thrombus is formed.

The search for anti-platelet drugs in the prevention of thrombosis has recently focused on the blockade of the GPIIb-IIIa receptor and on the inhibition of the vWF-GPIb axis. The best characterized drugs are antibodies and peptides that block the binding of adhesive proteins to GPIIb-IIIa that have been tested in animal models and of which many are being tested in clinical trials and/or are used in the clinic (J. E. Tcheng, Thromb. Haemost. 1997, 78(1):205-209; S. R. Hanson, K. S. Sakariassen, Am. Heart J. 1998, 135(5 Pt 2 Su):S132-S145; B. S. Coller, Thromb. Haemost. 1997, 78(1):730-735). Also compounds that interfere with the vWF-GPIb axis inhibit thrombus formation in various animal models. The GPIb/X/V complex consists of four different polypeptides GPIbα, GPIbβ, GPIX and GPV, which are all members of the leucine-rich repeat protein family (X. Du et al., Blood 1987, 69(5):1524-1527; and P. W. Modderman et al., J. Biol. Chem. 1992, 267(1):364-369). The N-terminal domain of the GPIbα polypeptide contains the vWF binding site (V. Vicente et al., J. Biol. Chem. 1988, 263(34):18473-18479). vWF is composed of several homologous domains, each covering different functions: it interacts through its A1 domain mainly with the GPIb/V/IX complex (M. C. Berndt et al., Biochemistry 1992, 31(45):11144-11151), whereas its A3 domain predominantly interacts with fibrillar collagen fibers (F. I. Pareti et al., J. Biol. Chem. 1987, 262(28):13835-13841; and H. Lankhof et al., Thromb. Haemost. 1996, 75(6):950-958). Compounds that interact with GPIbα, like the GPIb-binding snake venom proteins echicetin and crotalin (M. Peng et al., Blood 1993, 81(9):2321-2328; and M. C. Chang et al., Blood 1998, 91(5):1582-1589), an anti-guinea pig GPIb antibody (J. L. Miller et al., Arterioscler. Thromb. 1991, 11(5):1231-1236; and W. H. Dascombe et al., Blood 1993, 82(1):126-134), a recombinant A1 domain fragment (VCL) (A. I. McGhie et al., Circulation 1994, 90(6):2976-2981; and D. Zahger et al., Circulation 1995, 92(5):1269-1273), and recently an anti-human GPIb antibody (N. Cauwenberghs et al., Arterioscler. Thromb. Vasc. Biol. 2000, 20(5):1347-1353) or compounds that bind to vWF, like anti-A1-vWF-monoclonal antibodies (mAbs) (Y. Cadroy et al., Blood 1994, 83(11):3218-3224; and H. Yamamoto et al., Thromb. Haemost. 1998, 79(1):202-210), and aurin tricarboxylic acid (ATA) (P. Golino et al., Thromb. Haemost. 1995, 74(3):974-979) are inhibiting in vivo thrombus formation.

Specific blockade of the vWF-collagen interaction in vivo has for the first time been demonstrated by Hans Deckmyn et al., Anti-thrombotic von Willebrand factor (vwf) collagen bridging blockers, US20040071704 (2003 Nov. 21), GB200031448A 2000 Dec. 22, WO2001BE220 2001 Dec. 21 and US2003450740A 2003 Nov. 21, as a novel strategy for the prevention of thrombus formation in stenosed arteries. This study aimed to evaluate the anti-thrombotic efficacy of mAb 82D6A3 in baboons by using a modified Folts' model, where cyclic flow reductions (CFRs) due to thrombus formation and its dislodgement are measured in an artery following intimal damage and placement of a critical stenosis to reduce the lumen diameter (J. Folts, Circulation 1991, 83(6 Suppl):IV3-14).

Platelet adhesion to a damaged vessel wall is the first step in arterial thrombus formation. The tethering of platelets by vWF to the collagen exposed in the damaged vessel wall is especially important under high shear conditions. Anti-thrombotic compounds that interfere with the GPIb-vWF axis have been studied in animal models and were shown to be effective (N. Cauwenberghs et al., Arterioscler. Thromb. Vasc. Biol. 2000, 20(5):1347-1353; and H. Yamamoto et al., Thromb. Haemost. 1998, 79(1):202-210).

The present study evaluated for the first time the anti-thrombotic effects of inhibiting the vWF-collagen interaction in vivo. For this purpose, we used a monoclonal anti-human vWF antibody mAb 82D6A3 that by binding to the vWF A3-domain inhibits vWF binding to fibrillar collagens type I and III. mAb 82D6A3 furthermore cross-reacts with baboon vWF and inhibits baboon vWF binding to collagen type I under static and flow conditions (Depraetere et al., submitted). A modified Folts' model was used to evaluate the anti-thrombotic efficacy of mAb 82D6A3 under high shear conditions (J. Folts et al., Circulation 1991, 83(6 Suppl):IV3-14) in baboons. This model allows study of the cyclic flow reductions (CFRs) due to platelet-dependent thrombi forming at the injured, stenotic site of the artery. This cyclic flow model has been described as representing some of the events occurring in patients with unstable angina and useful for studying the mechanisms of unstable angina.

This model also allows a reproducible pattern of recurrent thrombosis to be established and is widely accepted as very effective and clinically relevant in testing potential anti-thrombotic agents (H. Ikeda et al., J. Am. Coll. Cardiol. 1993, 21(4):1008-1017; and J. T. Willerson et al., Proc. Natl. Acad. Sci. USA 1991, 88(23):10624-10628).

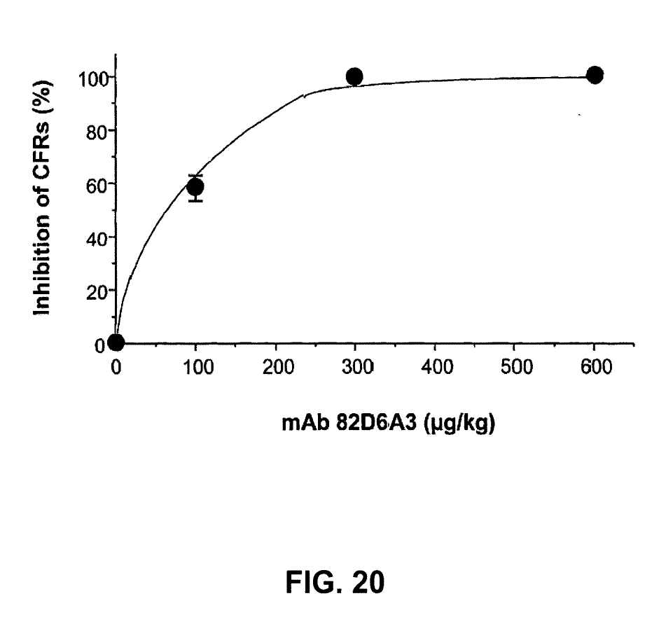

Administration of 100 μg/kg, 300 μg/kg and 600 μg/kg mAb 82D6A3 resulted in 58%, 100% and 100% inhibition of the CFRs, respectively (FIG. 20), which corresponded well with the 31%, 96% and 96% (measured in the 60-minute plasma samples) ex vivo inhibition of the vWF-collagen interaction (Tables IV and V).

None of the administered doses, even the highest one tested, 600 μg/kg, resulted in severe prolongation of the bleeding time or in thrombocytopenia (Table III), nor were the vWF-Ag levels impaired (Tables IV and V). These results, together with the ex vivo inhibition of the vWF-collagen interaction, show that the observed inhibitory effect results in a specific inhibition of the vWF-collagen interaction.

The absence of major bleeding problems correlates with our finding that the effect of mAb 82D6A3 on platelet adhesion to human collagen type I was more pronounced at higher shear rates. This confirms that the vWF-collagen interaction is especially important at high shear stress, in other words, in the arterial system, which could explain the observation of only a minor prolongation of the bleeding time.

The invention shows that inhibition of thrombus formation under high shear stress in vivo cannot only be obtained by inhibiting the vWF-GPIb interaction, but also by interfering with the vWF-collagen interaction. Both kinds of anti-thrombotics have the advantage of blocking the first step in thrombus formation, which might in addition have some beneficial action in preventing restenosis after PTCA or stenting, in contrast with specific GPIIb-IIIa blockers that only interfere after the platelets have been activated. Activated platelets do not only secrete platelet activating substances but also vasoactive compounds such as platelet-derived growth factor, known to induce smooth muscle cell migration and proliferation resulting in restenosis.

It was also revealed that F(ab)-fragments of 82D6A3, directed to the A3-domain of vWF, also bind to vWF with high affinity and are potent inhibitors of the vWF-collagen interaction under both static and flow conditions.

Selection of antibody binding phages from two different phage display libraries, a pentadecamer and cyclic hexamer library, resulted in phages that bind to 82D6A3 in a dose-dependent manner. Moreover, vWF and the recombinant A3-domain were able to inhibit phage binding to the MoAb indicating that the phages bind at or near to the antigen-binding site of 82D6A3. Sequence comparison of the phage-displayed peptides revealed that a consensus SPWR sequence was present in all phages selected. From these results, we can conclude that the SPWR sequence may be a part of the 82D6A3 epitope. The SPWR-sequence could be aligned to the VPWN sequence (aa 980-983) within the A3 domain, and in the three-dimensional structure of the A3-domain located in the vicinity of previously identified amino acid residues important for vWF-collagen interaction. Finding consistently the same four amino acid consensus sequence on the one hand indicates that this sequence really might be important in the antibody recognition. In conclusion, the invention demonstrates that vWF-collagen interaction plays an important role in acute platelet-dependent arterial thrombus formation: blockade of vWF-collagen interaction by mAb 82D6A3 or antigen-recognizing fragments thereof can induce complete abolition of thrombus formation in the injured and stenosed baboon femoral arteries. Accordingly, the mAb 82D6A3 can be used as a compound for the prevention of acute arterial thrombotic syndromes or to manufacture medicines to prevent acute arterial thrombotic syndromes.

### EXAMPLES

### Example 1

### Preparation and Purification of Intact Monoclonal Antibody 6B4, F(ab′)₂ and Fab Fragments Against GPIB

6B4 (subtype IgG1) is a murine monoclonal antibody raised against purified human GPIb and obtainable from the cell line deposited with the Belgian Coordinated Collections of Microorganisms under accession number LMBP 5108CB. When added at saturating concentrations, monoclonal antibody 6B4 totally abolishes both ristocetin- and botrocetin-induced human platelet aggregation as well as shear-induced platelet adhesion to human collagen type I tested in a Sakariassen-type flow chamber at 2600 s⁻¹.

Hybridoma cells producing the monoclonal antibody 6B4 were grown and subsequently injected into pristane (i.e., 2,6,10,14-tetramethyldecanoic acid)-primed Balb/c mice. After ten days, ascites fluid was collected. The immunoglobulin (IgG) was extracted from the ascites using protein-A-Sepharose CL-413 (available from Pharmacia, Roosendaal, Netherlands).

In order to prepare F(ab′)2 fragments, the monoclonal antibody 6B4 was dialyzed overnight against a 0.1 mol/l citrate buffer (pH 3.5). The antibody (200 parts) was digested by incubation with pepsin (one part) available from Sigma (St. Louis, Mo.) for one hour at 37° C. Digestion was stopped by adding one volume of a 1 M Tris HCl buffer (pH 9) to ten volumes of antibody.

Monovalent Fab fragments were prepared by papain digestion as follows: one volume of a 1 M phosphate buffer (pH 7.3) was added to ten volumes of the monoclonal antibody, then one volume papain (Sigma) was added to 25 volumes of the phosphate buffer containing monoclonal antibody, mmol/l L-Cysteine HCl (Sigma) and 15 mmol/l ethylene diamine tetra acetic acid (hereinafter referred to as “EDTA”). After incubation for three hours at 37° C., digestion was stopped by adding a final concentration of 30 mmol/l freshly prepared iodoacetamide solution (Sigma), keeping the mixture in the dark at room temperature for 30 minutes.

Both F(ab′)2 and Fab fragments were further purified from contaminating intact IgG and Fc fragments using protein-A-Sepharose. The purified fragments were finally dialyzed against phosphate-buffered saline (hereinafter referred as “PBS”). Purity of the fragments was determined by sodium dodecylsulphate polyacrylamide gel electrophoresis and the protein concentration was measured using the bicinchoninic acid Protein Assay Reagent A (Pierce, Rockford, Ill.).

### Example 2

### Method for Determining Deposition of Platelets

Autologous blood platelets were labeled with ¹¹¹In-tropolone and imaging and quantification of the deposition of ¹¹¹In-platelets were done as described by Kotze et al., J Nucl./Wed. (1991) 32:62-66. Briefly, image acquisition of the grafts, including proximal and distal silastic segments, was done with a Large Field of View scintillation camera fitted with a high resolution collimator. The images were stored on and analyzed with a Medical Data Systems A³ computer (Medtronic, Ann Arbor, Mich.) interfaced with the scintillation camera. Dynamic image acquisition, two-minute images (128×128 byte mode), was started simultaneously with the start of blood flow through the devices.

A two-minute image (128×128 byte mode) of a 3 ml autologous blood sample (collected in EDTA) was also acquired each time that the grafts were imaged to determine circulating blood radioactivity (blood standard). A region of interest of the graft segment was selected to determine the deposited and circulating radioactivity in each of the dynamic images. Radioactivity in a region of similar size of circulating radioactivity in the proximal segment of the extension tubing was determined, and subtracted from the radioactivity in the graft region to calculate deposited radioactivity. Platelet deposition was expressed as the total number of platelets deposited. The method to calculate this is described by Hanson et al., Arteriosclerosis (1985) 5:595-603.

### Example 3

### Receptor Binding Measurements

6B4, its F(ab′)2 or Fab fragments were labeled with Na¹²⁵I (Amersham, Buckinghamshire, UK) using the iodogen method as described by Fraker et al., Biochem. Biophys. Res. Comm. (1978) 80:849-857. Iodogen was purchased from Pierce (Rockford, Ill.). Platelet-rich baboon plasma, adjusted with autologous plasma to a count of 100,000 platelets/μl, was incubated with different concentrations of iodinated 6B4, F(ab′)2 or Fab fragments for 15 minutes at room temperature. The mixture was layered onto 20% sucrose buffer (wt/vol) containing 0.1% (wt/vol) bovine serum albumin (BSA) and centrifuged for four minutes at 10,000 g in Eppendorf tubes. The top fluid, including the plasma, was removed and the pellets were counted in a gamma-counter. This study was performed in duplicate on the platelet rich plasma of two baboons.

### Example 4

### In vitro and Ex Vivo Platelet Aggregation Measurement

The aggregation of platelets in response to ristocetin (1.5 mg/ml final concentration; abp, NY) was done on 10 ml blood collected in 1 ml of 3.2% trisodium citrate. Platelet-rich plasma was prepared by differential centrifugation as described by Van Wyk et al., Thromb. Res. (1990) 57:601-9, and the platelet count adjusted to 200,000 platelets/μl with autologous plasma. The aggregation response was measured in a Monitor IV Plus aggregometer (Helena Laboratories, Beaumont, Tex.) and recorded for five minutes. The percent aggregation at five minutes was calculated as the difference in light transmission between platelet-rich and platelet-poor plasma.

In in vitro studies, the platelet-rich plasma was preincubated for five minutes with serial dilutions of intact IgG 6B4, F(ab′)2 or Fab fragments before aggregation was initiated. Inhibition of aggregation was calculated from the difference in the aggregation response of platelets with and without antibody or fragments. In the ex vivo determinations, inhibition was calculated from the difference in the aggregation response of platelets before and after treatment of the baboons.

### Example 5

### Measurement of Plasma Concentrations of 6B4, F(ab′)2 or Fab Fragments and of Bleeding Time

Plasma concentrations were measured using a sandwich enzyme-linked immunoassay (ELISA). Briefly, microtiter plates were coated overnight at 4° C. with 5 μg/ml polyclonal goat anti-mouse IgG (Sigma). After blocking non-occupied binding sites with bovine serum albumin, serial dilutions of baboon plasma were added to the wells and incubated for two hours. Bound 6B4 (IgG, F(ab′)2 or Fab fragments) was detected by using goat anti-mouse IgG (Fab-specific) conjugated to peroxidase (Sigma). Standard curves were constructed by adding known amounts of 6B4 (IgG, F(ab′)2 or Fab fragments) to baboon plasma. Bleeding time was determined using the SIMPLATE® II device (Organon Teknika, Durham, N.C.) according to the instructions of the manufacturer, the volar surface of the forearm of the baboons being shaved and a pressure cuff being applied and inflated to 40 mm Hg.

### Example 6

### In Vitro Effect of Monoclonal Antibody 6B4 and Fab Fragments on Binding of vWF to Human GPIb Under Static and Flow Conditions

Monoclonal antibody 6B4 binds to a (1-289) recombinant (r) GPIbα fragment expressed by Chinese hamster ovary cells obtained from Meyer et al., J. Biol. Chem. (1993), 268:20555-20562, indicating that its epitope is localized within the amino-terminal region of GPIbα (=GPIbα).

Monoclonal antibody 6B4 Fab fragments were further tested for inhibition of ristocetin- and botrocetin-induced binding of vWF to the rGPIbα (recombinant GPIBα) fragment using an ELISA set-up, as described by Vanhoorelbeke et al., Thromb. Haemost. (2000):83:107-113. Microtiter plates were coated with 5 μg/ml monoclonal antibody 2D4 for 48 hours at 4° C. Monoclonal antibody 2D4, another anti-GPIb monoclonal antibody, binds to the rGPIb-fragment but does not block vWF binding. Non-adsorbed sites were blocked with 3% skimmed milk, whereafter the plates were washed with tris-buffered saline (hereinafter referred as “TBS”) containing 0.1% TWEEN® 20 (TBS-Tw). Purified rGPIb-fragments were immobilized on monoclonal antibody 2D4 by incubating 2 μg/ml rGPIbα for two hours at 37° C. After washing with TBS-Tw, increasing concentrations of 6B4 Fab fragments (diluted in TBS-Tw) were added, followed by 1.25 or 0.6 μg/ml purified human vWF (available from the Red Cross Belgium), respectively, when ristocetin (300 μg/ml) or botrocetin (0.5 μg/mL) were used as modulators. Binding of vWF was determined by incubating for one hour with HRP-conjugated polyclonal anti-vWF antibody (Dako, Glostrup, Denmark), diluted 1/3000 in TBS-Tw. The color reaction, stopped with 4 mol/L H₂SO₄ was generated with orthophenylenediamine (available from Sigma). The purification of botrocetin from crude Bothrops jararaca venom (available from Sigma) was performed according to Fujimura et al., Biochemistry (1991) 30:1957-1964.

The effect of 6B4 Fab fragments on shear-induced platelet adhesion to collagen was tested in a Sakariassen-type parallel-plate flow chamber at shear rates of 650, 1,300 and 2,600 sec-1, according to Harsfalvi et al., Blood (1995), 85:705-7011. Human collagen type I (Sigma) was dissolved in 50 mM acetic acid (1 mg/ml), dialyzed for 48 hours against PBS and subsequently sprayed onto plastic Thermanox coverslips and stored at room temperature overnight before use.

12 ml of blood, anti-coagulated with LMW heparin (25 U/mL, Clexane, Rhône-Poulenc Rorer, France), was preincubated with 6B4 Fab fragments at 37° C. for five minutes and then used to perfuse the collagen-coated coverslips. After five minutes of perfusion, the platelets were fixed with methanol and the coverslips stained with May-Grünwald Giemsa. Platelet adhesion (percent of total surface covered with platelets) was evaluated with a light microscope connected to an image analyzer. An average of 30 fields per cover slip were analyzed. Platelet adhesion was expressed as percentage maximal platelet adhesion obtained in the absence of inhibitor.

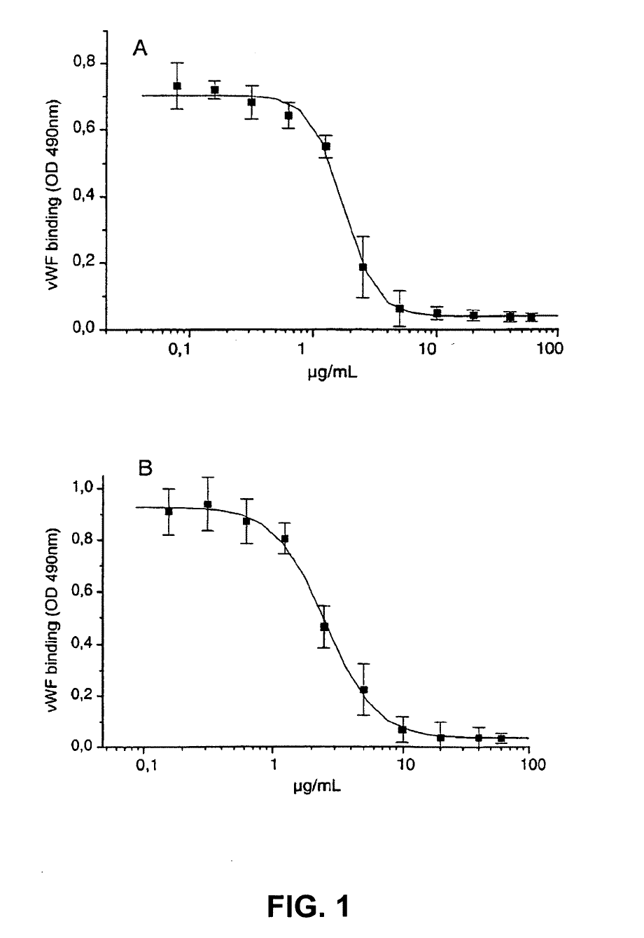

Monoclonal antibody 6B4 Fab fragments block the ristocetin-(1 mg/ml) and botrocetin-(0.5 μg/ml) induced human platelet agglutination with an IC50 of 1.2±0.3 μg/ml (24±6 nmol/L) and 2.0±0.5 μg/ml (40±10 nmol/L), respectively. 6B4 binds to an epitope localized on the amino-terminal part (His 1-Val 289) of GPIbα. As shown in FIG. 1, the 6B4 Fab fragments dose dependently inhibited both the ristocetin- and botrocetin-induced binding of vWF to rGPIb, with an IC50 of 1.8 μg/ml (36 nmol/L) and 2.5 μg/ml (50 nmol/L), respectively, when the binding was induced by ristocetin (300 μg/ml) or botrocetin (0.5 μg/ml).

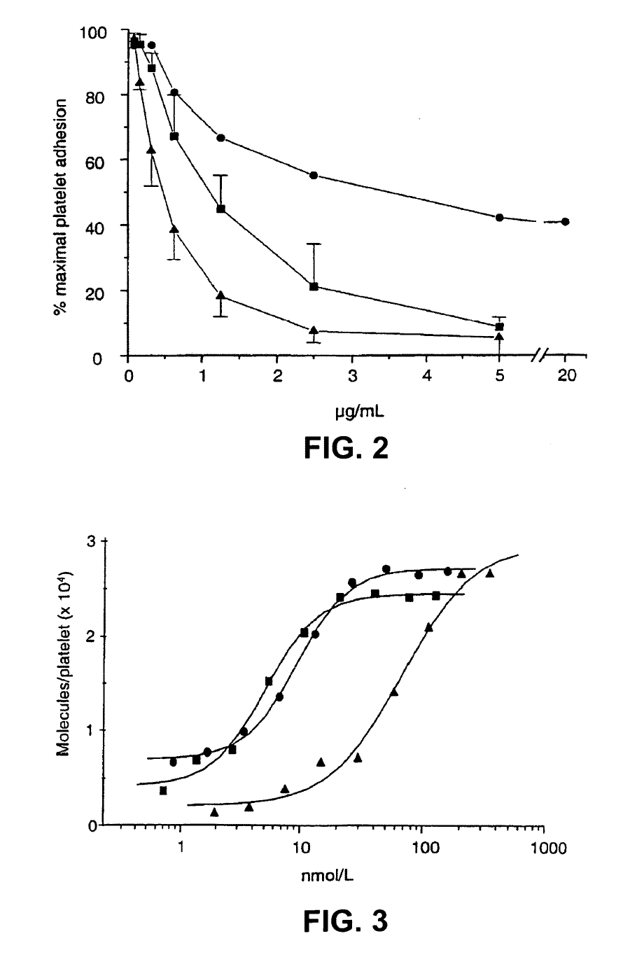

As shown in FIG. 2, the 6B4 Fab fragments inhibited platelet adhesion to collagen type I in a concentration-dependent manner at shear rates of 650, 1,300 and 2,600 sec⁻¹. A 50% reduction of surface coverage was obtained at a concentration of 3.5 μg/ml (70 nmol/l), 1.1 μg/ml (22 nmol/l) and 0.5 μg/ml (10 nmol/L), respectively, for shear rates of 650, 1,300 and 2,600 sec⁻¹.

### Example 7

### In Vivo Studies in Baboons: Dose Response Effect of 6B4 Fab-Fragments on Platelet Adhesion and Deposition

Male baboons (Papio ursinus) weighing between 10 and 15 kg and being disease-free for at least six weeks, were used according to procedures approved by the Ethics Committee for Animal Experimentation of the University of the Orange Free State (South Africa) and the National Code for Animal Use in Research, Education, Diagnosis and Testing of Drugs and Related Substances (South Africa). The baboons supported permanent TEFLON®-Silastic Arteriovenous (AV) shunts implanted in the femoral vessels according to Hanson et al. (cited supra). Blood flow through the shunts varied between 100 and 120 ml/minute, resulting in wall shear rates between 800 and 1,000 sec⁻¹, which compares with the shear rates found in medium-sized arteries. Handling of the baboons was achieved through anesthesia with about 10 mg/kg ketamine hydrochloride (Anaket-V, Centaur Laboratory, South Africa).

In order to test the effect of the monoclonal antibody on platelet count, 6B4 and its F(ab′)2 and Fab fragments were administered to three different baboons. The injected dose was calculated to attain a plasma concentration of 1×KD₅₀, i.e., the concentration needed to occupy 50% of the receptors as determined in in vitro experiments.

Platelet-dependent arterial thrombus formation was induced by using bovine pericardium (0.6 cm²) fixed in buffered glutaraldehyde according to the method disclosed by Quintero et al., J Heart Valve Dis. (1998), 7:262-7. The pericardium was built into the wall of silicone rubber tubing (3 mm inside diameter). The method of preparation of the thrombogenic device is described by Kotze et al., Thromb. Haemost. (1993), 70:672-5, except that fixed bovine pericardium instead of DACRON® vascular graft material was used. In each experiment, a thrombogenic device, prefilled with saline to avoid a blood-air interface, was incorporated as an extension segment into the permanent AV-shunt by means of TEFLON® connectors as previously disclosed by Hanson et al. (cited supra).

In this first approach to determine the effect of 6B4 fragments on platelet adhesion, seven baboons were used and thirteen perfusion experiments were performed. In the first five experiments (three baboons), a thrombogenic device was placed to determine deposition of platelets according to the method of Example 2. After 30 minutes, the device was removed and blood flow through the permanent AV-shunt reestablished. Fifteen minutes after removal of the device, each baboon was treated with a bolus of 80 μg/kg Fab fragments of 6B4 (in 2 ml saline) and, again fifteen minutes later, a second thrombogenic device was placed for 30 minutes to determine the effect of the Fab fragments on thrombogenesis. The device was again removed and blood flow through the permanent shunt established. This was followed by a second bolus injection of Fab fragments (80 μg/kg) to attain a cumulative dose of 160 μg/kg. After fifteen minutes, a third thrombogenic device was placed for 30 minutes and platelet deposition measured according to the method of Example 2. In four other experiments (two baboons), the same study protocol was used but two doses of 320 μg/kg were administered.

In four other experiments (four baboons), sham studies were performed by using the same protocol of placement of thrombogenic devices, but the baboons were not treated with Fab fragments.

Blood was collected at different periods of time (given in the figures) to determine platelet count and hematocrit (EDTA), circulating and platelet-associated radioactivity, the ex vivo aggregation of platelets in response to ristocetin (according to the method of Example 4), and the plasma concentrations of Fab fragments (according to the method of Example 5).

### Example 8

### In Vivo Studies in Baboons: Effect of Anti-GPIb 6B4 Fragments on Interplatelet Cohesion

In this second approach to determine the effect of 6B4 fragments on interplatelet cohesion, six baboons were selected in a manner similar to that of Example 7 and used as follows. In all baboons, a thrombogenic device was placed for 24 minutes. In six experiments (three baboons), the baboons received a bolus injection of Fab fragments of 110 μg/kg. The fragments were injected six minutes after placement of the thrombogenic device to allow enough platelets to be deposited to cover the collagen surface. In the six other experiments, the other three baboons did not receive Fab fragments.

As in Example 7, blood was collected at different periods of time (given in the figures) to determine platelet count and hematocrit (EDTA), circulating and platelet associated radioactivity, the ex vivo aggregation of platelets in response to ristocetin (according to the method of Example 4), and the plasma concentrations of Fab fragments (according to the method of Example 5).

FIG. 3 shows binding curves of anti-GPIb ¹²⁵I-6B4 IgG (▪), -F(ab′)2 () and -Fab fragments (▴) to baboon platelets in plasma. Binding of the antibody and its fragments to baboon platelets was dose-dependent and saturable: half saturation (KD₅₀) was obtained with 4.7 nmol/l, 6.4 nmol/l and 49.2 nmol/l for the monoclonal antibody 6B4 IgG, its F(ab′)2 and Fab fragments, respectively.

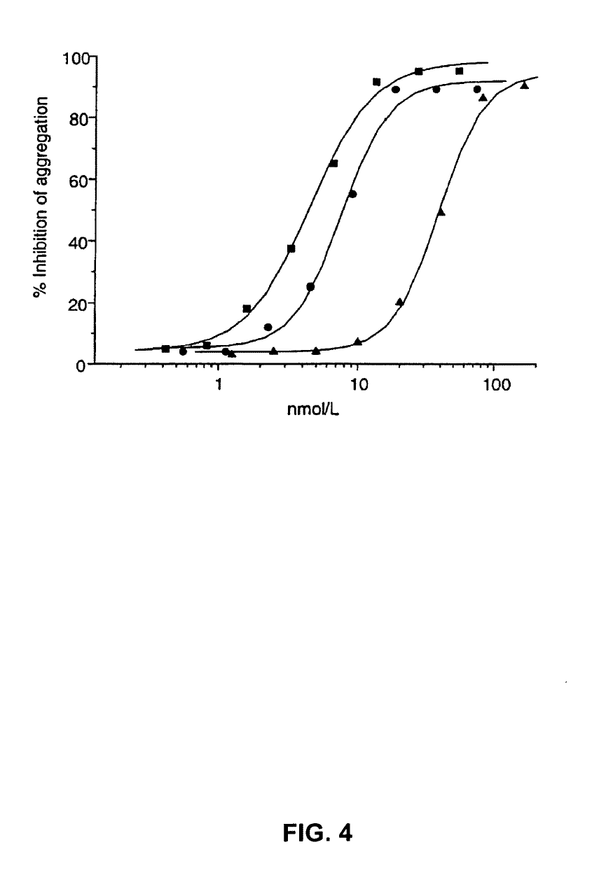

FIG. 4 shows the inhibitory effect of anti-GPIb 6B4 IgG (▪), -F(ab′)2 () and -Fab fragments (▴) on ristocetin-induced baboon platelet aggregation. When added at saturating concentrations, ristocetin-induced aggregation was completely abolished: IC₅₀-values were 4.5 nmol/l, 7.7 nmol/l and 40 nmol/l for the monoclonal antibody 6B4 IgG, its F(ab′)2 and Fab fragments, respectively.

When considering the effect of injection of the monoclonal antibody 6B4, F(ab′)2 and Fab fragments on the peripheral platelet count in baboons, the dose of the 6B4 and its fragments used were calculated, for purposes of comparison to attain a plasma concentration of 1×KD₅₀. In one baboon, 100 μg/kg of intact antibody caused a profound decrease in the blood platelet count (<30×10⁹ pl/l) within ten minutes after injection. After 48 hours, the platelet count was still below 100×10⁹ pl/l. When 6B4 F(ab′)2 fragments were injected into two baboons, the platelet count decreased rapidly to between 120 and 150×10⁹ pl/l, i.e., by approximately 60%, and then reached pre-infusion values within 24 hours. Finally, when 80 to 320 μg/kg of the monovalent 6B4 Fab fragments was injected, the platelet count (45 minutes after injection) decreased only by approximately 10 to 20% and by 26% when 640 μg/kg was injected as shown in Table 1 hereinafter.

FIG. 5 shows platelet deposition onto thrombogenic devices containing bovine pericardium placed consecutively at times 0 (), 60 (▪), and 120 (▴) minutes for 30 minutes (top shaded bars). For panel A, sham experiments, for Panel B, after injection of 0 (), 80 (▪), 160 (▴), 320 (♦) and 640 (▾) μg/kg 6B4 Fab fragments. In the sham studies (FIG. 5, Panel A), placement of the previous graft had no significant effect on platelet deposition formed on subsequent grafts. In the treatment studies (FIG. 5, Panel B), dosages of 80 μg/kg and 160 μg/kg significantly inhibited platelet deposition in comparison to control, by approximately 43% and 53%, respectively. Doses of 320 μg/kg and 640 μg/kg significantly reduced platelet deposition by 56% and 65%, respectively.

Plasma levels of 6B4 Fab-fragments and inhibition of ex vivo agglutination determined on samples obtained 45 minutes or two hours after administration both changed dose- and time-dependently, as shown in Table 1 hereinafter.

Bleeding times, determined in the treatment studies before and 45 minutes after injecting 80 to 320 μg/kg of 6B4 Fab fragments, were not significantly prolonged. Only a dose of 640 μg/kg significantly prolonged the bleeding time, which was still less than doubled.

FIG. 6 shows the influence of late treatment of baboons with 6B4 Fab fragments on platelet deposition, the thrombogenic device being placed at time 0 and platelet deposition determined for 24 minutes (top shaded bar). After six minutes (arrow), baboons were either untreated (▪) or treated with a bolus of 110 μg/kg () 6B4 Fab fragments. It is thus shown that 110 μg/kg 6B4 Fab fragment did not affect platelet deposition when injected after a thrombus was allowed to form for an initial six minutes.

Interpretation of experimental results: The anti-GPIb monoclonal antibody 6B4, its F(ab′)2 and Fab fragments potently inhibited the binding of vWF to a recombinant GPIbα fragment (His1-Val289) and dose-dependently inhibited vWF-dependent human platelet agglutination. The intact monoclonal antibody and its fragments also dose-dependently inhibited human platelet adhesion to type I collagen in a flow chamber at wall shear rates of 650, 1300 and 2600 sec⁻¹. This inhibition was shear-dependent, i.e., more pronounced at higher shear.

6B4, its F(ab′)2 and Fab fragments also bind to and inhibit baboon platelets with much the same characteristics as human platelets. As a result, baboons were used for in vivo and ex vivo studies. An almost immediate, profound and irreversible thrombocytopenia developed when the intact antibody was injected into a baboon, similar to what was observed when other anti-GPIb monoclonal antibodies were injected into different experimental animals. The F(ab′)2 fractions also caused immediate, but reversible thrombocytopenia, but to a lesser extent than the intact antibody. The Fab fractions, on the other hand, had only a moderate effect on the blood platelet count, which strongly suggests that the Fc portion of the monoclonal antibody plays a part in the development of the irreversible thrombocytopenia.

The 6B4 Fab fractions were used to assess an anti-thrombotic effect in a baboon model of arterial thrombosis. The glutaraldehyde-fixed bovine pericardium was highly thrombogenic: after 30 minutes of exposure to native flowing blood, approximately 3×10⁹ platelets deposited on the area of 0.6 cm².

In similar studies, only approximately 0.7×10⁹ platelets accumulated on Dacron vascular graft material (0.9 cm²) according to Kotze et al., Thromb. Haemost. WO 01/10911.23 PCT/IEP00/07874 (1993) 70:672-675. It is, therefore, not surprising that a number of control thrombogenic devices occluded before 30 minutes of exposure to flowing blood.

Treatment of baboons with 6B4 Fab fragments inhibited platelet deposition on the thrombogenic devices by between 43% and 65%. The observed effect must be ascribed to the monoclonal antibody, since sequential placement of thrombogenic devices in untreated baboons caused no decreased deposition. No complete inhibition of platelet deposition was observed, even at high doses.

### Example 9

### In Vivo Studies in Baboons: Effect of 6B4 Fab Fragments on Cyclic Flow Variations in Stenosed, Endothelium-Injured Arteries

The experimental model used herein is adapted from the model originally described by J. D. Folts et al., in Circulation (1982), 65:248-255, as a canine model of coronary artery stenosis with intimal damage. Basically, this model allows the study of cyclic flow reductions in coronary blood flow due to platelet-dependent thrombi forming at the site of a coronary stenosis that was created by the placement of a fixed constrictor. It provides a reproducible pattern of recurrent thrombosis to be established and is widely accepted as very effective and clinically relevant in testing potential anti-thrombotic agents.

Our adaptation is such that the model was set-up in one femoral artery of the baboons, since the 6B4 Fab fragments do not cross-react with canine platelets.

A. Surgical Preparation and Study Protocol

Normal baboons (Papio ursinus) weighing 10 to 15 kg, disease-free for at least six weeks before the experiments, were used. All experiments were approved by the Ethics Committee for Animal Experimentation of the University of the Free State in accordance with the National Code for Animal Use in Research, Education, Diagnosis and Testing of Drugs and Related Substances in South Africa. Baboons were anesthetized with ketamine hydrochloride (=10 mg/kg IM; Anaket-V, Centaur Laboratory). The intra-arterial pressure was continuously monitored throughout the procedure. Blood for the laboratory tests (Examples 3 through 5) was obtained from one of the femoral veins.

First, a calibrated electromagnetic flow probe was placed around the proximal portion of the isolated femoral artery of the baboons in order to measure arterial blood flow. After the animal was allowed to stabilize for approximately 30 minutes, the endothelium of the femoral artery was injured by gently squeezing with forceps, and cyclic flow reductions due to platelet-dependent thrombus formation were induced by placement of a constrictor.

When flow declined to near zero, blood flow through the constricted femoral artery was restored by manually shaking the constrictor. The cyclic pattern of decreasing arterial blood flow following restoration was referred to as cyclic flow reductions, and this pattern was continuously monitored for 60 minutes.

The baboons in which the cyclic flow reductions were studied, were divided into three groups. One group (two baboons) received a placebo (saline solution), the second group (four baboons) was treated with a bolus injection of 600 μg/kg 6B4 Fab fragments and the third group (three baboons) received an injection of 2 mg/kg 6B4 Fab fragments. In addition, 4 mg/kg 6B4 Fab fragments were injected in one baboon in order to determine the effect of such a high dose on platelet count, receptor occupation, bleeding time and platelet aggregation but cyclic flow reductions were not followed in this baboon.

Animals instrumented to produce cyclic flow reductions were treated with 6B4 Fab fragments or placebo after a 30-minute baseline monitoring period. Cyclic flow reductions were continuously monitored in each animal during 60 minutes. The anti-thrombotic effect was quantified by comparing the frequency of cyclic flow reductions per hour before and after drug administration. Blood samples for the different laboratory measurements (platelet count, hematocrit, platelet aggregation, receptor occupation and plasma levels) were drawn at several periods in time: before the 60-minute monitoring period and, respectively, 30, 60, 150, 300 minutes and 24 hours after treatment.

B. Results

1. Effect of 6B4 Fab Fragments on Cyclic Flow Reductions (FIG. 7)

In the baboons that received a placebo injection of saline solution, the frequency of the cyclic flow reductions (CFR) at 60 minutes after injection was not changed (107±7%) significantly as compared to the pre-treatment control period. A dose of 600 μg/kg 6B4 Fab fragments resulted in a partial inhibition of the cyclic flow reductions, reducing their frequency to 41±15%. A dose of 2 mg/kg completely abolished the cyclic flow reductions in all three animals studied and this inhibition (6±6%) was observed throughout the 60-minute study period. Heart rate, blood pressure and hematocrit remained unchanged during the study.

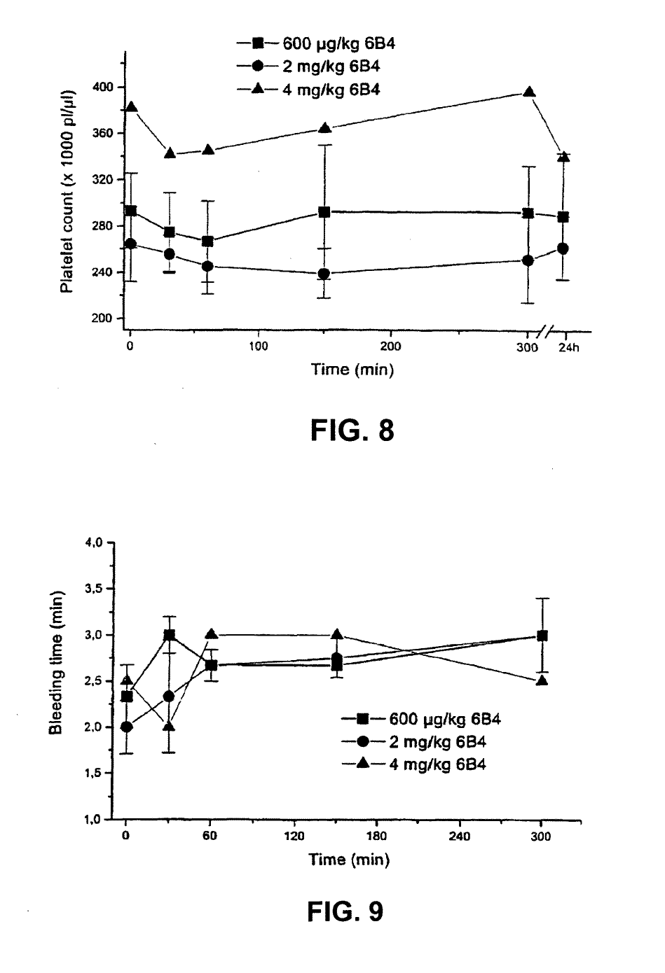

2. Effect of 6B4 Fab Fragments on Platelet Count and Bleeding Times (FIGS. 8 and 9)

The platelet count (FIG. 8) was not significantly affected by injection of 600 μg/kg, 2 mg/kg or 4 mg/kg of the 6B4 Fab fragments. Also, the bleeding time (FIG. 9) was not significantly prolonged by injection of 600 μg/kg, 2 mg/kg or 4 mg/kg of the 6B4 Fab fragments.

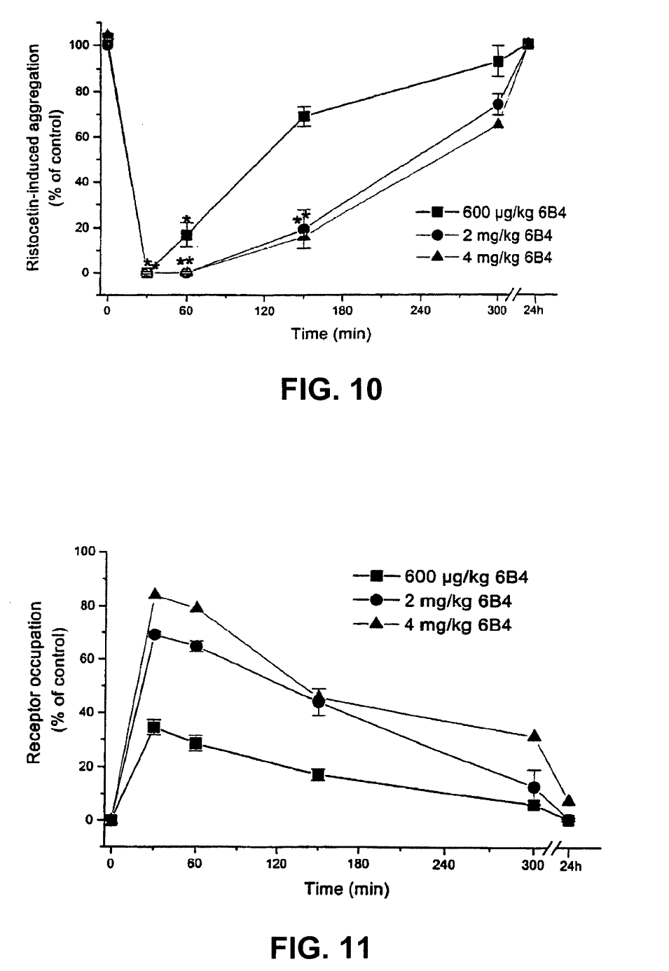

3. Inhibition of Ex Vivo Platelet Aggregation (FIG. 10)

6B4 Fab fragments inhibited the ex vivo ristocetin-induced platelet aggregation in a dose- and time-dependent manner when administered to the baboons (FIG. 10). Aggregation was totally abolished 30 minutes after injection and, as compared to the aggregation response before injection, aggregation was significantly (p<0.05) reduced to 16.8%, 5.2% and 2% 60 minutes and to 68.8% (p>0.05), 19.2% and 16% 150 minutes after a bolus injection of 600 μg/kg, 2 and 4 mg/kg 6B4 Fab fragments, respectively. The inhibitory effect lasted for about 150 minutes and returned to normal values within 24 hours.

4. Receptor Occupancy (FIG. 11)

The occupancy of GPIb receptors by the 6B4 Fab fragments is shown in FIG. 11. Thirty minutes following a bolus injection of 600 μg/kg, 2 and 4 mg/kg 6B4 Fab fragments, approximately 34.5%, 69.3% and 84% of the GPIb receptors were occupied, respectively. The receptor occupancy was 28.6%, 64.8% and 79% after 60 minutes; 17.1%, 43.9% and 45.6% after 150 minutes; and dropped to 6.3%, 12.9% and 31.3% after 300 minutes following injection of, respectively, 600 μg/kg, 2 and 4 mg/kg 6B4 Fab fragments. The decrease in receptor occupancy corresponds with the time course of the ex vivo ristocetin-induced aggregation results.

C. Interpretation of Experimental Results

Platelet adhesion, activation and aggregation play a pivotal role in the development of coronary artery syndromes. In particular, the high shear stress present in the constricted coronary arteries is an important initiator of the platelet activation and aggregation. Several investigators have shown that cyclic flow reductions in stenosed damaged canine coronary arteries can be prevented by metabolic inhibition of platelet activation, or by blockade of the GPIIb/IIIa receptor.

In this study we have shown that administration of Fab fragments of the inhibitory anti-GPIb monoclonal antibody 6B4 is effective in diminishing or abolishing cyclic flow variations in stenosed, endothelium-injured femoral arteries in non-human primates. The presumed mechanism by which this occurs is the inhibition of the interaction of the platelet glycoprotein Ib receptor and the vessel wall-bound von Willebrand factor. This prevents platelet activation and aggregation as well as the release of pro-aggregatory and vasoconstrictor substances responsible for these cyclic flow variations.

6B4 Fab fragments completely abolished the cyclic flow reductions at a dose of 2 mg/kg, and reduced them by 59% after injection of 600 μg/kg. Bleeding times were not significantly prolonged, even when injecting 4 mg/kg of the 6B4 fragments, suggesting that 6B4 Fab fragments are a useful anti-thrombotic agent with low bleeding risk. Moreover, there was no fall in platelet count, again indicating that injection of the 6B4 fragments is not expected to cause any hemostatic problems. In vivo administration of the 6B4 Fab fragments resulted in a dose- and time-dependent inhibition of ex vivo ristocetin-induced platelet aggregation and correlated with the receptor occupancy. The duration of the effects of the 6B4 Fab fragments persisted for about three hours when a dose that completely abolished the cyclic flow reductions (>2 mg/kg 6B4 Fab fragments) was given, with receptor occupancy and anti-platelet effects (ristocetin-aggregation) returning to baseline values about six hours after injection. In conclusion, 6B4 Fab fragments demonstrate the desired properties to be promising compounds for the treatment of acute coronary syndromes with a low bleeding risk.

### Example 10

### Cloning and Sequencing of Monoclonal Antibody 6B4

In order to reduce the possible immunogenicity of the murine anti-GPIb monoclonal antibody 6B4, it may be necessary to construct chimeric antibodies combining the variable region of the mouse antibody with a human antibody constant region. Depending on the antibody, such chimeric antibodies have been found from substantially reducing to little affecting the immunogenic response. Further humanization by complementary determining region-grafting or resurfacing usually has proven to be a successful approach. In order to produce such humanized antibodies, a first step is to determine the sequence of the murine antibody.

Cloning of Variable Region cDNAs

Total RNA from approximately 3×10⁷ 6B4-hybridoma cells grown in 75 mL T-flasks was prepared using the Qiagen RNeasy Midi Procedure (Westburg) following the manufacturers' instructions and next quantitated by an OD260 reading. cDNA was synthesized from the total RNA by incubating 2 μg of tRNA with 1 μM of poly(dT)₁₅ adaptor primer and 4 U of Omniscript reverse transcriptase in a total volume of 20 μl with other reaction buffers and following incubation times as recommended by the manufacturer (Qiagen Omniscript RT Kit) (Westburg).

Next, the V genes were amplified for cloning into the pCRII-TOPO® vector (TOPO TA-Cloning® Kit, In Vitrogen) for sequence determination.

Polymerase chain reaction amplification was done using V_{H} back (5′-CAGGTSMARCTGCAGSAGTCWGG-3′ (SEQ ID NO:5)) and V_{H} for (5′-TGAGGAGACGGTGACCGTGGTCCCTTGGCCCCAG-3′ (SEQ ID NO:6)), V_{L} back (5′-GACATTGAGCTCACCCAGTCTCCA-3′ (SEQ ID NO:7)) and V_{K2} for (5′-GGAAGCTTGAAGATGGATACAGTTGGTGCAGC-3′ (SEQ ID NO:8)) primers with M, R, W and S, respectively, (A/C), (A/G), (A/T) and (C/T) (all from Eurogentec, Herstal, Belgium) and V_{H} back. V_{H} for and V_{L} back are complementary to the 5-terminal part of the framework region FR-1 and to the 3′-terminal part of the FR-4 of the V_{H}- and V_{L}-genes, respectively, and V_{K2} for anneals to the C_{K} sequence.

Polymerase chain reactions were performed in a programmable heating block using 30 rounds of temperature cycling (92° C. for one minute, 55° C. for one minute and 72° C. for one minute). The reactions included the cDNA, 1 μg of each primer and 2.5 U of Hotgold polymerase (Eurogentec) in a final volume of 50 μl, with the reaction buffers as recommended by the manufacturer (Vitrogen). The polymerase chain reaction product bands were analyzed on a 1.5% agarose gel.

Transformation was done using the heat-shock method and using E. coli TOP-10 cells (TOPO TA-Cloning™ Kit, In Vitrogen) according to the manufacturer's instructions. The cells from each transformation were plated onto LB+ampicillin). Transformation was checked by polymerase chain reaction amplification of the inserts and next analyzed on a 1.5% agarose gel. Positive clones were grown up for purification of plasmid DNA by the Qiagen Maxi plasmid purification kit.

Sequencing reactions were performed with the ABI Prism Big Dye terminator cycle Sequencing Ready Reaction kit (Perkin Elmer Applied Biosystems, Netherlands) according to the manufacturer's instructions using M13 Forward primer 5′-TTCCTCGACGCTAACCTG-3′ (SEQ ID NO:9) and M13 Reverse primer 5′-GATTTAATCTGTATCAGG-3′ (SEQ ID NO:10) and which align to the pCRII-TOPO™ vector.

### Results and Interpretation

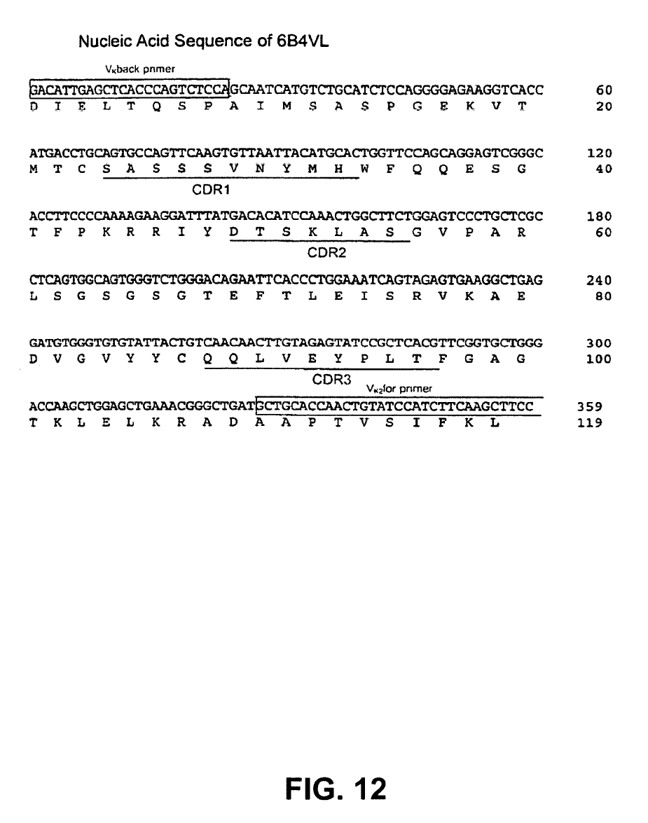

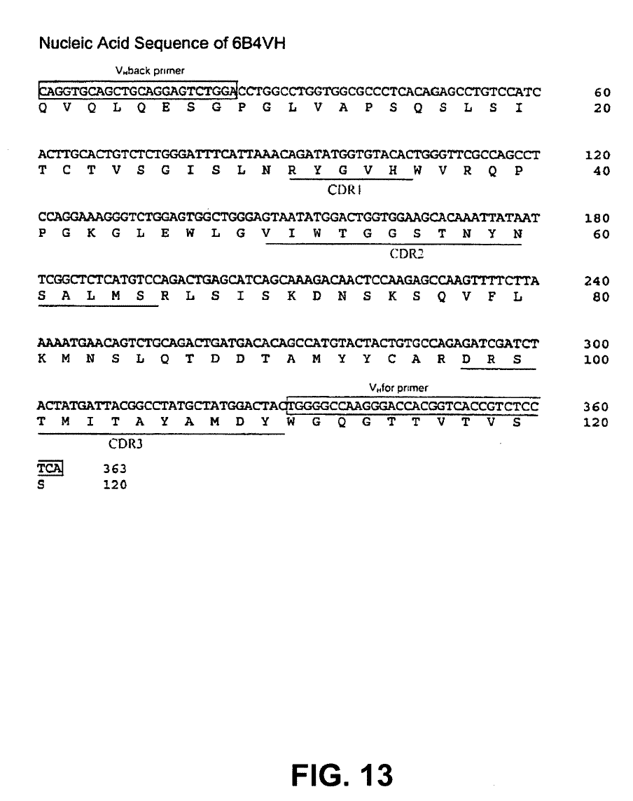

The cDNA from the heavy-chain variable domain genes were amplified by polymerase chain reaction using primers that hybridize to the framework regions FR-1 and FR-4. For amplification of the light-chain variable domain genes, we used a primer that hybridizes to FR-1 and one that anneals in the constant region. These V_{H} and V_{L} genes were next cloned into the pCRII-TOPO™ vector and transformed into E. coli TOPO™ cells. By using appropriate primers, the V_{H} and V_{L} genes were next sequenced. The translated amino acid sequences are given in FIGS. 12 (light chains) and 13 (heavy chains), respectively, and the six complementary-determining regions conferring epitope specificity are indicated in these figures. The heavy chain (V_{H}) revealed a sequence closely related to mouse heavy chain subgroup Ib, whereas the light chain NO gene sequence matches to mouse K-chain subgroup V. Given our choice of priming sites, it is not possible to determine the exact sequence at both ends of the V genes, as it is dictated by the primer (amino acid residues 1-8 of FR1 of the V_{L}, and residues 1-8 of FR1 of the V_{H}, and 111-121 of FR4 of the V_{H}). Nevertheless, these uncertainties in the framework regions are unlikely to affect antigen specificity since this is determined by the complementary-determining regions

As a principal finding, we demonstrate here that selective blockade of key pathways mediating platelet adhesion and aggregation has different impacts on stroke outcome. Our study shows for the first time that interfering with early steps of platelet-vessel wall interactions mediated by GPIb and GPVI reduces stroke severity after transient middle cerebral artery occlusion (tMCAO). Anti-GPIbα Fab treatment had a very strong protective effect when performed both before and after tMCAO. This suggests a central role of this receptor in the pathogenesis of ischemic stroke, whereas GPVI may have a significant but less prominent function. This is in agreement with the model that GPIbα is mandatory for the initial attachment of platelets to the vessel wall under conditions of elevated shear, whereas GPVI serves mainly as an activating receptor, a function that also can be fulfilled by alternative pathways, most notably G protein-coupled receptors (Z. M. Ruggeri et al., Nat. Med. 2002, 8:1227-1234; and B. Nieswandt et al., J. Thromb. Haemost. 2005, 3:1725-1736). Importantly, stroke protection after anti-GPIbα Fab or anti-GPVI mAb treatment was not accompanied by intracranial bleeding complications.

In contrast, application of F(ab)₂ targeting the GPIIb/IIIa receptor had no positive effect on stroke outcome but significantly increased the rate of intracerebral bleedings and mortality in a dose-dependent manner. The cerebral microvasculature rapidly responds to brain ischemia (G. J. del Zoppo, T. Mabuchi, J. Cereb. Blood Flow Metab. 2003, 23:879-894). Endothelial cells up-regulate cell adhesion molecules, and endothelial denudation of vessels exposes subendothelial matrix proteins such as collagen to the bloodstream. Recently, two important pathways have been described that facilitate early adhesion of platelets to vessel walls: binding of the platelet surface receptor GPIb to endothelial vWF (R. K. Andrews et al., Thromb. Res. 2004, 114:447-453) and adhesion of platelets to collagen via their GPVI receptor (B. Nieswandt and S. P. Watson, “Platelet-collagen interaction: is GPVI the central receptor?” Blood 2003, 102:449-461).

In accordance with our findings in experimental stroke, inhibition of either platelet GPIb or vWF reversed flow reductions after experimental femoral artery stenosis (N. Cauwenberghs et al., Arterioscler. Thromb. Vasc. Biol. 2000, 20:1347-1353; D. Wu et al., Arterioscler. Thromb. Vasc. Biol. 2002, 22:323-328; D. Wu et al., Blood 2002, 99:3623-3628; and S. Kageyama et al., Eur. J. Pharmacol. 2002, 443:143-149). Importantly, reduced stroke volumes after GPIb inhibition in our study were accompanied by a significant reduction in neurological deficits even when anti-GPIbα Fab was injected with a delay of one hour after the induction of tMCAO. This underlines the functional significance of this novel therapeutic approach and indicates its potential suitability for clinical application during the acute phase of ischemic stroke in humans, in whom treatment options are very limited. Interestingly, polymorphisms of platelet GPIbα exist, and variants that lead to enhanced vWF/GPIb interactions are associated with an increased risk of ischemic stroke in humans (R. I. Baker et al., Blood 2001, 98:36-40; and A. P. Reiner et al., Stroke 2000, 31:1628-1633). Similarly, increased serum levels of vWF represent an independent stroke risk factor (T. N. Bongers et al., Stroke 2006, 37:2672-2677).

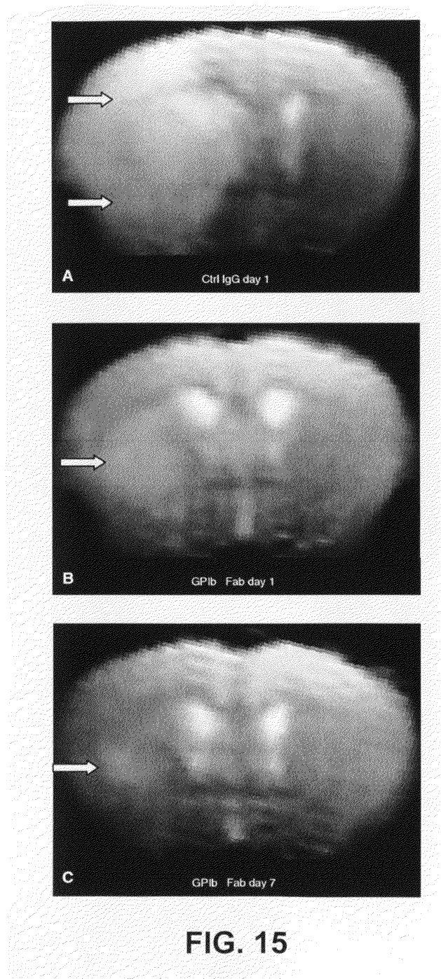

Although tail bleeding times are strongly elevated after treatment of mice with anti-GPIbα Fab fragments, no increase in intracranial hemorrhage (ICH), which represents the main obstacle for anti-thrombotic therapy during the acute stroke phase in clinical practice, was detected. This surprising finding suggests that the mechanisms by which platelets prevent intracranial bleeding are different from those involved in the sealing of a tail bleeding wound. Both processes are clearly platelet-dependent because platelet depletion or virtually complete inhibition of GPIIb/IIIa results in both markedly prolonged tail bleeding times and intracranial bleeding after tMCAO (FIG. 15, Panel A). Thus, our results strongly confirm the previous finding that no direct correlation exists between bleeding time and bleeding risk (R. P. Rodgers and J. Levin, “A critical reappraisal of the bleeding time,” Semin. Thromb. Hemost. 1990, 16:1-20).

The initial loose adhesion of platelets to the damaged endothelium is followed by firm attachment, which is mediated through the platelet collagen receptors (R. K. Andrews and M. C. Berndt, “Platelet physiology and thrombosis,” Thromb. Res. 2004, 114:447-453). Among the numerous collagen receptors expressed in platelets, GPVI is of central importance for cellular activation and subsequent firm arrest (B. Nieswandt et al., “Glycoprotein VI but not alpha2beta1 integrin is essential for platelet interaction with collagen,” EMBO. J. 2001, 20:2120-2130). Treatment of mice with anti-GPVI antibodies specifically and persistently depletes GPVI from platelets (B. Nieswandt et al., “Long-term anti-thrombotic protection by in vivo depletion of platelet glycoprotein VI in mice,” J. Exp. Med. 2001, 193:459-469; and V. Schulte et al., “Targeting of the collagen-binding site on glycoprotein VI is not essential for in vivo depletion of the receptor,” Blood 2003, 101:3948-3952). Several reports have demonstrated a profound anti-thrombotic effect of GPVI inhibition after artificial arterial wall injury and collagen-induced thromboembolism (S. Massberg et al., J. Exp. Med. 2003, 197:41-49; B. Nieswandt et al., J. Exp. Med. 2001, 193:459-469; and S. Goto et al., Circulation 2002, 106:266-272).

We now show that treatment of mice with the anti-GPVI antibody JAQ1 significantly reduced the brain infarct volumes at day I after tMCAO. This indicates that platelet/collagen interactions via GPVI also may be involved in stroke development. GPVI depletion was less effective than GPIb blockade and did not significantly affect clinical outcome variables. However, it is well established that significant reductions in stroke volumes on histological examination after MCAO often do not translate into measurable clinical improvement (F. Wahl et al., Stroke 1992, 23:267-272). Although tail bleeding times were slightly increased by JAQ1, intracerebral bleeding frequency and mortality after 24 hours were not altered, indicating a favorable safety profile.

The different extent of stroke protection in favor of GPIb blockade may be due to a more general role of GPIb in stroke development. GPIb, but not GPVI, additionally mediates leukocyte adhesion to attached platelets by a Mac-1-dependent pathway (R. K. Andrews and M. C. Berndt, Thromb. Res. 2004, 114:447-453; and R. K. Andrews et al., Int. J. Biochem. Cell. Biol. 2003, 35:1170-1174). Furthermore, resting platelets can bind to the activated endothelium via GPIb interaction with P-selectin (M. Berndt et al., Thromb Haemost. 2001, 86:178-188). Both, leukocyte adhesion and P-selectin up-regulation have been shown to contribute to stroke development, probably by impairing reperfusion of the cerebral microvasculature (G. J. del Zoppo et al., J. Cereb. Blood Flow Metab. 2003, 23:879-894; and T. V. Arumugam et al., Am. J. Physiol. Heart Circ. Physiol. 2004, 287:2555-2560).

GPIIb/IIIa antagonists inhibit the final common pathway of platelet aggregation, regardless of the agonist that stimulates platelet activation (R. K. Andrews et al., Thromb. Res. 2004, 114:447-453). In agreement with previous reports (S. Gruner et al., Blood 2003, 102:4021-4027), antibody-mediated blockade of the GPIIb/IIIa receptor had no significant effect on peripheral platelet counts but completely inhibited ex vivo platelet aggregation in response to different stimuli and resulted in tail bleeding times consistently >20 minutes in our study (Table II). Impaired hemostasis after >95% GPIIb/IIIa blockade could explain the high frequency of ICH and mortality after tMCAO in our study. A substantial risk of ICH has previously been reported after tMCAO in mice treated with the GPIIb/IIIa inhibitor SDZGPI562 (T. F. Choudhri et al., J. Clin. Invest. 1998, 102:1301-1310).

The unexpected high rate of bleeding complications is consistent with similar observations during a recent phase III, double-blind, placebo-controlled, multicenter study testing the safety and efficacy of abciximab in ischemic stroke. This clinical study was stopped prematurely because of significantly increased ICH and mortality, as well as lack of efficacy (H. P. Adams and W. Hacke for the AbESTT-II Investigators, Abciximab in Emergent Stroke Treatment Trial-II (AbESTT-II): results of a randomized, double-blind placebo-control phase 3 study, presented at 15th European Stroke Conference, May 19, 2006, Brussels, Belgium; Abstract 1).

In the preceding phase II trial, treatment with abciximab had shown a nonsignificant shift in favorable outcomes only (Abciximab Emergent Stroke Treatment Trial (AbESTT) Investigators, Emergency administration of abciximab for treatment of patients with acute ischemic stroke: results of a randomized phase 2 trial, Stroke 2005, 36:880-890). Several experimental studies reported a beneficial effect of GPIIb/IIIa antagonists on stroke size and functional outcome (T. F. Choudhri et al., J. Clin. Invest. 1998, 102:1301-1310; M. Maeda et al., J. Cereb. Blood Flow Metab. 2005, 25:108-118; and A. Moriguchi et al., J. Cereb. Blood Flow Metab. 2005, 25:75-86). GPIIb/IIIa antagonists also have been successfully used in experimental and clinical settings in conjunction with recombinant tissue-type plasminogen activator thrombolysis therapy without major complications reported (G. Ding et al., J. Cereb. Blood Flow Metab. 2005 25:87-97; and U. Junghans et al., Neurology 2002, 58:474-476). This is in contrast to our study in which no effect on stroke volume or functional deficit was observed, regardless of the anti-GPIIb/IIIa F(ab)₂ dosage used.

Increasing evidence exists, however, that the extent of GPIIb/IIIa inhibition may be critical, which could explain the divergent experimental and clinical observations. At peak concentrations, GPIIb/IIIa inhibitors may effectively act as platelet antagonists accompanied by increased bleeding complications, whereas subthreshold GPIIb/IIIa antagonism may lead to platelet activation and thrombus formation (D. L. Bhatt and E. J. Topol, Nat. Rev. Drug Discov. 2003, 2:15-28). In line with this, a 67.8% or 78.4% GPIIb/IIIa receptor blockade was safe (but ineffective) in our experimental stroke model, whereas complete inhibition substantially increased ICH and mortality. Taken together, it appears that GPIIb/IIIa antagonists have a very narrow therapeutic window, limiting their clinical use at least in cerebral ischemia (D. L. Bhatt and E. J. Topol, Nat. Rev. Drug Discov. 2003, 2:15-28; and P. A. Ringleb, Stroke 2006, 37:312-313) in contrast to their proven utility in percutaneous coronary artery interventions, which, however, is age dependent (D. L. Bhatt and E. J. Topol, JAMA 2000, 284:3124-3125; and G. Ndrepepa et al., Circulation 2006, 114:2040-2046).

Currently, not enough evidence exists from randomized controlled trials on the efficacy or safety of GPIIb/IIIa inhibitor therapy in acute stroke or its use combined with thrombolysis (A. Ciccone et al., Stroke 2007, 38:1113-1114). The devastating consequences of ICH in patients require a particularly high safety profile for any anti-platelet therapy or anti-coagulation during stroke because, depending on location, even small bleedings can cause major neurological deficits. Our present study provides evidence that blocking of the platelet receptor GPIb involved in platelet adhesion can diminish infarct development in mice and, because of a lack of ICH, may open new avenues for acute stroke treatment in humans in the future.

Table 1. Platelet counts, plasma levels of 6B4 Fab-fragments, ex vivo ristocetin-induced platelet agglutination and bleeding times following administration of 80 to 640 μg/kg 6B4 Fab fragments to baboons. Values are given as mean±SE. Statistical comparisons were made using student t-test for paired sample groups (p<0.05).

TABLE 1
Platelet counts, plasma levels of 6B4 Fab-fragments, ex vivo ristocetin-induced platelet agglutination
and bleeding times following administration of 80-640 μg/kg 6B4 Fab fragments to baboons. Values
are given as mean ± SE. Statistical comparisons were made using student t-test for paired sample
groups (*p < 0.05).

| | | | Platelet | | | % Inhibition of ex vivo | |
|---|---|---|---|---|---|---|---|
| Dose | | Time | counts | | Plasma levels | ristocentin-induced (1.5 mg/mL) | Bleeding |
| (μg/kg) | n | (min) | (×10³/μL) | (% decrease) | (μg/mL) | platelet agglutination | times (sec) |
| 0 | 5 | Pre | 307 ± 32 | (0) | 0.07 ± 0.03 | 0 | 190 ± 20 |
| 80 | 5 | 90 | 272 ± 22 | (11) | 1.72 ± 0.14 | 26 ± 9 | 160 ± 33 |
| 160 | 5 | 150 | 248 ± 19 | (19) | 4.84 ± 0.56 | 47 ± 12* | 250 ± 45 |
| | | 270 | 315 ± 31 | | 0.45 ± 0.09 | 8 ± 3 | ND |
| 0 | 4 | Pre | 283 ± 23 | (0) | 0.02 ± 0.01 | 0 | 232 ± 42 |
| 320 | 4 | 90 | 219 ± 10 | (23) | 9.13 ± 0.48 | 25 ± 21 | 340 ± 63 |
| 640 | 4 | 150 | 210 ± 13 | (26) | 15.35 ± 1.38 | 80 ± 9* | 405 ± 45* |
| | | 270 | 238 ± 20 | (16) | 1.19 ± 0.09 | 15 ± 9 | ND |
| | | 24 h | 236 ± 13 | (17) | 0.04 ± 0.01 | 7 ± 3 | ND |

ND non determined

### Example 11

### Anti-Platelet Antibodies on a Mouse Stroke Model

### Materials

All monoclonal antibodies (mAbs) were produced, characterized, and modified in our laboratories as described previously in detail. The following mAbs were used: mAbs against mouse GPIIb/IIIa (JON/A) (W. Bergmeier et al., Cytometry 2002, 48:80-86) GPIbα (p0p/B) (S. Massberg et al., J. Exp. Med. 2003, 197:41-49), and GPVI (JAQ1) (B. Nieswandt et al., “Expression and function of the mouse collagen receptor glycoprotein VI is strictly dependent on its association with the FcRgamma chain,” J. Biol. Chem. 2000, 275:23998-24002). Fab and F(ab)₂ fragments were prepared as described (B. Nieswandt et al., “Identification of critical antigen-specific mechanisms in the development of immune thrombocytopenic purpura in mice,” Blood 2000, 96:2520-2527). Briefly, antibodies were incubated for six to eight hours with immobilized papain or for 24 hours with immobilized pepsin according to the manufacturer's instructions (Pierce Biotechnology, Inc, Rockford, Ill.), and the preparations were then applied to an immobilized protein A column, followed by an immobilized protein G column (Pharmacia, Freiburg, Germany) to remove Fc fragments and undigested IgG. Purity of Fab or F(ab)₂ fragments was tested by SDS-PAGE. For control experiments, purified rat IgG2a (Serotec, Darmstadt, Germany) and nonimmune control rat IgG Fab were used.

### Antibody Administration

To inhibit GPIbα, mice received 100 μg p0p/B Fab IV one hour before or one hour after transient middle cerebral artery occlusion (tMCAO). GPIIb/IIIa receptors were blocked by injection of 100 μg (>95% receptor blockade; see Table II), 20 μg (78.4% receptor blockade), or 10 μg (67.8% receptor blockade) JON/A F(ab)₂ IV one hour before the start of the experiment. To inhibit GPVI function, mice received 100 μg JAQ1 IP five days before infarct induction. Mice had at that time point no detectable GPVI in platelets for at least five more days (B. Nieswandt et al., “Long-term anti-thrombotic protection by in vivo depletion of platelet glycoprotein VI in mice,” J. Exp. Med. 2001, 193:459-469). Two different groups of control animals received either 100 μg purified rat IgG2a or 100 μg rat IgG Fab.

TABLE II
Effects of Selective Anti-Platelet Antibodies on Hemostasis in Mice

| | | | 100 μg | 20 μg | 10 μg | |
|---|---|---|---|---|---|---|
| | Rat IgG | GPIbα | GPIIb/IIIa | GPIIb/IIIa | GPIIb/IIIa | GPVI |
| | (Control) | Fab | F(ab)₂ | F(ab)₂ | F(ab)₂ | mAbs |
| Receptor | 0 | >95 | >95 | 78.4 ± 4.8 | 67.8 ± 6.1 | Depletion |
| occupancy, % | | | | | | |
| Platelet count, | 1.00 ± 0.20 | 0.91 ± 0.18 | 1.04 ± 0.08 | 0.90 ± 0.14 | 1.02 ± 0.12 | 0.98 ± 0.11 |
| ×10⁻⁶/μL | | | | | | |
| Bleeding time, | 4.4 ± 3.8 | 9.3 ± 5.8 | . . . | ND | ND | 7.3 ± 4.6 |
| min | | | | | | |
| Mice bleeding | 1/16 | 6/14 | 12/12 | | | 0/12 |
| >20 min, n/N | | | | | | |

### Example 12

### Stroke Model Experiments

### Bleeding Time Experiments

To determine bleeding times, mice were anesthetized, and a 3-mm segment of the tail tip was amputated with a scalpel. The tail was then blotted with filter paper every 15 seconds until the paper was no longer blood stained (P. Carmeliet et al., J. Clin. Invest. 1993, 92:2756-2760). When necessary, bleeding was manually stopped after 20 minutes to prevent death.

### Animal Studies

Animal studies were approved by the Regierung von Unterfranken and conducted according to the recently published recommendations for research in mechanism-driven basic stroke studies (U. Dirnagl, “Bench to bedside: the quest for quality in experimental stroke research,” J. Cereb. Blood Flow Metab. 2006, 26:1465-1478). Adult male C57/BL6 mice (20 to 25 g) were purchased from Charles River (Sulzfeld, Germany). The tMCAO model was used to induce focal cerebral ischemia as described in detail elsewhere (C. Kleinschnitz et al., J. Exp. Med. 2006, 203:513-518; and V. M. Clark et al., Neurol. Res. 1997, 19:641-648). Briefly, mice were anesthetized with 2% isoflurane in a 70% N₂O/30% O₂ mixture. A servo-controlled heating blanket was used to maintain core body temperature close to 37° C. throughout surgery. After a midline neck incision was made, a standardized silicon rubber-coated 6.0 nylon monofilament (60-1720RE, Doccol, Redlands, Calif.) was inserted into the right common carotid artery and advanced via the internal carotid artery to occlude the origin of the MCA. After one hour, mice were reanesthetized, and the occluding filament was removed to allow reperfusion. All animals were operated on by the same operator (C.K.) to reduce infarct variability; operation time per animal did not exceed 15 minutes.

After recovery from anesthesia and again after 24 hours, neurological function was assessed by two blinded investigators. Global neurological status was scored according to Bederson et al. (J. B. Bederson et al., Stroke 1986, 17:472-476). Motor function and coordination were graded with the grip test (P. M. Moran et al., Proc. Natl. Acad. Sci. U.S.A. 1995, 92:5341-5345).

Laser Doppler flowmetry (Moor Instruments, Devon, UK) was used to monitor regional cerebral blood flow in the MCA territory in antibody-treated animals and controls before surgery (baseline), immediately after MCA occlusion, and again five minutes after removal of the occluding monofilament (reperfusion) (C. Kleinschnitz et al., J. Exp. Med. 2006, 203:513-518; and E. S. Connolly Jr. et al., Neurosurgery 1996, 38:523-531). After the thread was advanced, regional cerebral blood was reduced by 95±3% and recovered to 68±5% of baseline (100%) after removal of the filament, indicating sufficient occlusion and reperfusion of the vessel beds. Values did not differ statistically between the groups at any time point (not shown).

After the right femoral artery was punctured, blood gases (PO₂, PCO₂, pH) were analyzed during the operation in three animals per group (antibody-treated mice or controls). The measured values were within the physiological range and showed no significant differences (not shown).

### Determination of Infarct Size and ICH

Mice were killed 24 hours after tMCAO. Brains were quickly removed and cut into 2-mm-thick coronal sections using a mouse brain slice matrix. The slices were stained with 2% 2,3,5-triphenyltetrazolium chloride (TTC; Sigma-Aldrich, Seelze, Germany) in PBS to visualize the infarctions. Planimetric measurements (ImageJ software, National Institutes of Health, Bethesda, Md.) were performed by researchers blinded to the treatment group and were used to calculate lesion volumes, which were corrected for brain edema as described (R. A. Swanson et al., J. Cereb. Blood Flow Metab. 1990, 10:290-293).

The occurrence of ICH was macroscopically assessed on whole brains and again after the 2-mm-thick coronal brain slices were cut (see above) before TTC staining. Brains showing ICH were excluded from the assessment of infarct volumes.

### Stroke Assessment by Magnetic Resonance Imaging

To investigate the frequency of ICH over time after tMCAO, magnetic resonance imaging (MRI) was performed repeatedly at an early stage (24 hours) and at seven days after stroke on a 1.5-T MR unit (Vision, Siemens, Berlin, Germany) under inhalation anesthesia as described previously (C. Kleinschnitz et al., J. Exp. Med. 2006, 203:513-518). For all measurements, a custom-made dual-channel surface coil designed for the examination of mice head was used (A063HACG, Rapid Biomedical, Würzburg, Germany). The image protocol comprised a coronal T2-weighted sequence (slice thickness, 2 mm) and a coronal 3D T2-weighted gradient-echo constructed interference in steady state (slice thickness, 1 mm) sequence. MRIs were visually assessed by researchers blinded to the prior treatment with respect to infarct morphology and, in particular, the occurrence of ICH.

### Statistical Analysis

Results are presented as mean±SD. The frequency of ICH and the mortality rate at day 1 were compared between groups with the χ² test. Infarct volumes and functional data were tested for Gaussian distribution with the D'Agostino and Pearson omnibus normality test and then analyzed by Bonferroni-corrected 1-way ANOVA. For statistical analysis, PrismGraph 4.0 software (GraphPad Software, San Diego, Calif.) was used. Values of P<0.05 were considered statistically significant.

### Example 13

### Stroke Results: GPIb and GPVI Blockade Improves Stroke Outcome after Transient Cerebral Ischemia

To study the involvement of platelets in the development of ischemic stroke, we inhibited the function of platelet membrane receptors involved in primary adhesion and activation. Complete blockade of GPIbα was achieved by intravenous injection of 100 μg Fab fragments of the monoclonal antibody p0p/B. In contrast to the intact IgG (B. Nieswandt et al., Blood 2000, 96:2520-2527), the Fab is not cytotoxic and, therefore, had no significant influence on platelet counts. After one hour, receptor occupancy was >95% as shown by flow cytometric analysis of p0p/B^{FITC} binding (Table II). Tail bleeding times in anti-GPIbα-treated mice were markedly prolonged, with six of fourteen mice being unable to stop bleeding within the 20-minute observation period and the other eight mice showing a strong prolongation compared with control IgG- or control Fab-treated animals. In parallel, mice were treated with the anti-GPVI antibody JAQ1 (100 μg IP) and analyzed on day 5. As previously described (B. Nieswandt et al., J. Exp. Med. 2001, 193:459-469), these animals lacked detectable GPVI in circulating platelets but displayed normal platelet counts and only very moderately increased tail bleeding times (Table II).

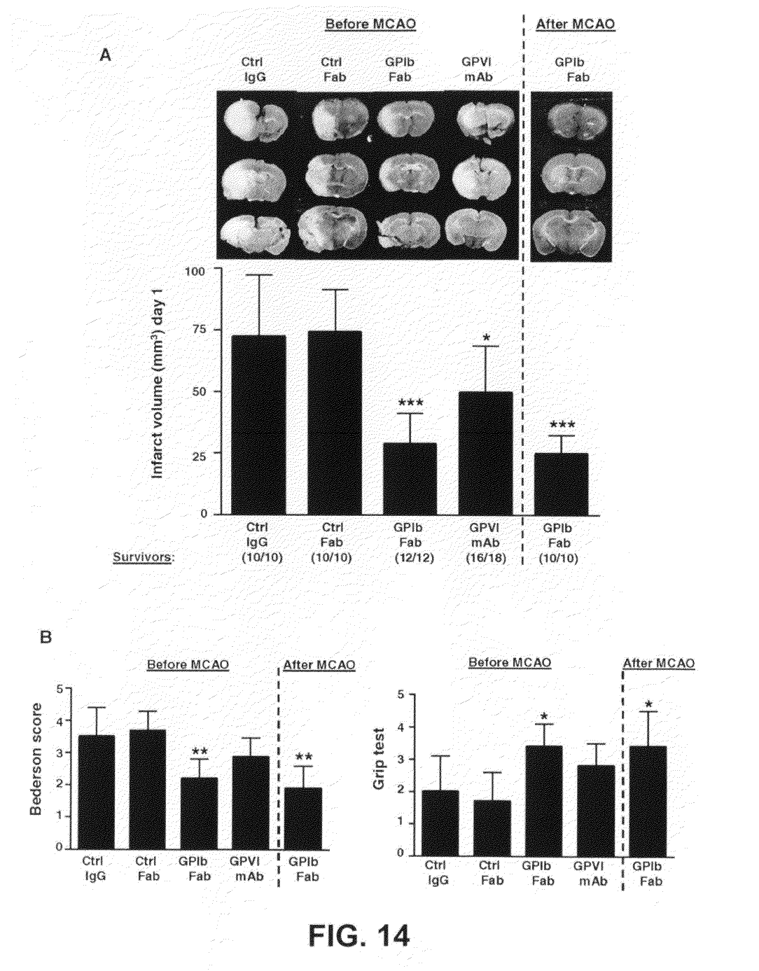

Mice treated with anti-GPIbα Fab one hour before or anti-GPVI mAbs five days before challenge were subjected to transient (60 minutes) cerebral ischemia and analyzed after 24 hours. At that time point, infarct volumes were reduced dramatically in anti-GPIbα-treated mice to ≈40% compared with controls (28.5±12.7 versus 73.9±17.4 mm3; P<0.001; FIG. 14, Panel A). Similarly, depletion of GPVI significantly diminished the infarct volume but to a lesser extent (49.4±19.1 mm3; P<0.05; FIG. 14, Panel A). Reduction in infarct size after anti-GPIbα treatment was functionally relevant in that the Bederson score assessing global neurological function and the grip test that specifically measures motor function and coordination were significantly better than in controls (FIG. 14, Panel B) (Bederson score, 3.7±0.6 versus 2.2±0.6, respectively; P<0.001; grip test, 1.7±0.9 versus 3.4±0.7, respectively; P<0.01). Anti-GPVI-treated mice tended to develop less severe neurological deficits compared with controls, but the differences did not reach statistical significance (FIG. 14, Panel B). To test whether GPIb blockade also is beneficial in the acute phase after focal cerebral ischemia, anti-GPIbα Fab was applied one hour after the induction of tMCAO. Indeed, this therapeutic approach was as effective as prophylactic anti-GPIbα infusions before tMCAO because brain infarct volumes were reduced to a comparable extent (24.5±7.7 mm3; P<0.001; FIG. 14, Panel A) and the neurological status again was improved (Bederson score, 1.9±0.7; P<0.001; grip test, 3.4±1.1; P<0.01; FIG. 14, Panel B). Taken together, these results indicate that the platelet receptors GPIb and GPVI may contribute critically to stroke development after tMCAO.

We next analyzed the impact of platelet inhibition on the occurrence of intracerebral bleeding complications after experimental cerebral ischemia. Mice receiving anti-GPIbα Fab or anti-GPVI mAbs did not show hemorrhagic transformation of infarcted brain regions as assessed by morphological analysis (not shown). Accordingly, mortality rates were not increased compared with IgG or Fab controls (not shown). This important notion could be confirmed by serial MRI studies. In animals treated with anti-GPIbα Fab, ischemic infarcts always appeared hyperintense on T2-weighted MRI. There were no additional hypointense areas indicating hemorrhage on days 1 and 7 after tMCAO when blood-sensitive T2-weighted gradient-echo MR sequences were used. These MRI findings exclude the occurrence of ICH (FIG. 15).

GPIIb/IIIa Blockade is Ineffective in tMCAO and Dose-Dependently Increases the Risk of ICH

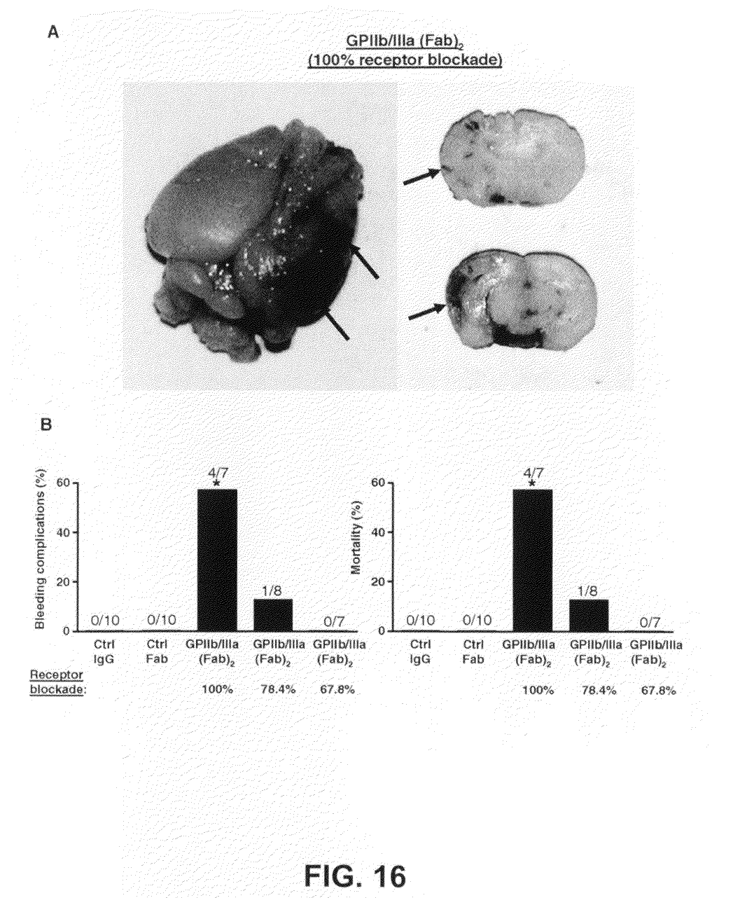

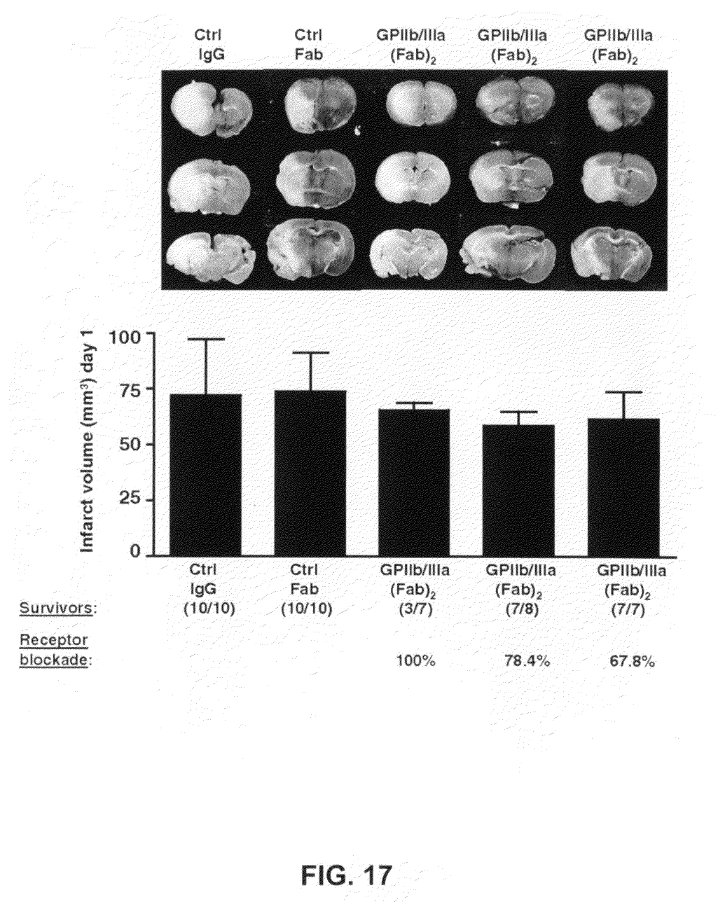

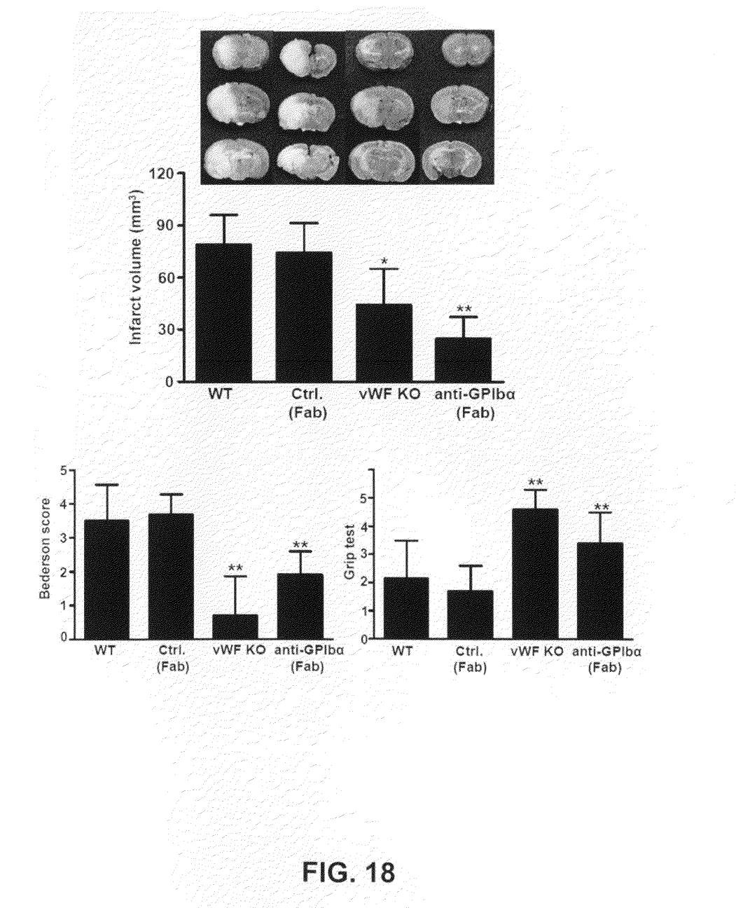

We next asked whether blockade of the final common pathway of platelet aggregation via GPIIb/IIIa would effectively reduce infarct volumes even more after tMCAO. Unexpectedly, four of the seven animals that had received 100 μg anti-GPIIb/IIIa F(ab)₂ leading to a virtually complete receptor blockade (see Table II) died as a result of ICH (P<0.05; FIG. 16, Panels A and B), and the three surviving animals exhibited infarct volumes of the same extension as controls (65.7±3.4 mm3; P>0.05; FIG. 17). The high incidence of ICH was very similar to that in mice in which platelets had been depleted by >98% by injection of cytotoxic anti-GPIb antibodies (not shown) (B. Nieswandt B et al., Blood 2000, 96:2520-2527).

To analyze whether the risk of ICH after GPIIb/IIIa blockade is dose dependent and to further evaluate the efficacy of anti-GPIIb/IIIa F(ab)₂ treatment in experimental stroke, additional groups of mice received 20 or 10 μg anti-GPIIb/IIIa F(ab)₂, which led to a 78.4% and 67.8% receptor blockade, respectively (see Table II). In contrast to complete GPIIb/IIIa inhibition, only one of fifteen animals developed ICH and died (FIG. 16, Panel B), but both concentrations failed to influence infarct volumes (58.8±6.3 and 61.6±12.4 mm3; P>0.05; FIG. 17) or neurological outcome (Bederson score, 3.0±0.6 and 3.2±0.9; P>0.05; grip test, 2.0±0.8 and 2.3±1.0; P>0.05).

### Example 14

### Materials, Preparations and Experimental of the Anti-vWF Studies

### Materials for the VWF Inhibition Study

Human placental collagen type I and III and calfskin type I were purchased from Sigma (St. Louis, Mo.). The collagen were solubilized in 50 nmol/L acetic acid and subsequently dialyzed against phosphate-buffered saline PBS (48 hours, 4° C.) to obtain fibrillar collagen. The phage display library with the random hexapeptides flanked by cysteine residues was obtained from Corvas (Gent, Belgium), the pentadecamer phage display peptide library was a kind of gift of Dr. G. Smith (University of Missouri, Colombia, Mo.). vWF was purchased from Red Cross (Belgium). The Spl proteolytic fragment and recombinant A3-domain were kind gifts of Drs. J. P. Girma (INSERM 134, Paris) and Ph. G. de Groot (Utrecht, The Netherlands).

### Purification of mAb 82D6A3

mAb 82D6A3 was obtained from a cell line that has been deposited with the Belgium Collection of Microorganisms under accession number LMBP 5606CB and was purified from ascites by protein A chromatography.

### Preparation of 82D6A3 F(ab) Fragment

2D6A3-F(ab) was prepared by digestion with papain. Briefly, 5 mg Ab was digested with 50 μg papain (Sigma) in the presence of 10 mmol/L cysteine and 50 mmol/L EDTA (37° C., overnight). The F(ab) was purified by protein A affinity chromatography (Pharmacia Roosendaal, The Netherlands) and purity was checked by SDS-PAGE.

### Surgical Preparation

Seven baboons of either sex, weighing 12 to 18 kg, were used in the present study. The experimental procedure followed was a modification of the original Folts' model (J. Folts, “An in vivo model of experimental arterial stenosis, intimal damage, and periodic thrombosis,” Circulation 1991, 83(6 Suppl):IV3-14). Baboons were anesthetized with ketamine hydrochloride (10 mg/kg, i.m.), intubated with a cuffed endotracheal tube and ventilated by a respirator with oxygen supplemented with 0.5% Fluothane to maintain anesthesia. Body temperature was maintained at 37° C. with a heating table. A catheter was placed in a femoral vein for drug administration and blood sampling. A segment of another femoral artery was gently dissected free from surrounding tissue and a perivascular ultrasonic flow probe (Transonic Systems Inc., New York, N.Y.) was placed around the distal dissection site. The mean and phasic blood flows were recorded continuously throughout the experiment. Baboons were allowed to stabilize for 30 minutes.

Then, the proximal dissection site of the femoral artery was injured by applying three occlusions of the artery for ten seconds with 2 mm interval using a spring-loaded forceps. A spring-loaded clamp next was placed in the middle of the injured site to produce an external stenosis of 65% to 80%. A gradual decline in blood flow due to platelet adhesion and aggregation was observed. When flow reached zero, blood flow was restored by pushing the spring of the clamp to mechanically dislodge the platelet-rich thrombus. This repetitive pattern of decreasing blood flow following mechanical restoration was referred to as cyclic flow reductions (CFRs). Additional endothelial injury and appropriate external stenosis selection was repeated. Finally, stable CFRs were obtained in these baboons.

After a 60-minute control period of reproducible CFRs (t=60 minutes to 0 minutes), test agents (saline or mAb 82D6A3) were given via an intravenous bolus injection (t=0) and monitoring was continued up to 60 minutes after drug administration (t=+60 minutes). The anti-thrombotic effect was quantified by comparing the number of CFRs per hour before and after drug administration. Blood samples for the different laboratory measurements (platelet count, coagulation, vWF occupation, vWF-collagen binding and plasma levels) were drawn at t=0, +30, +60, +150, +300 minutes and 24, 48 hours after treatment.

### Drug Treatment

The doses of mAb 82D6A3 were selected on the basis of preliminary dose finding studies. In group I, two baboons were used as saline control. Three baboons, group II, received a dose of 0.1 mg/kg mAb 82D6A3, after 60 minutes recording, an additional 0.2 mg/kg mAb 82D6A3 was given. Since a preliminary study showed that mAb 82D6A3 has a long half-life, this, therefore, resulted in a final dose of 0.3 mg/kg. In group III, a dose of 0.6 mg/kg mAb 82D6A3 was given to two baboons. All agents were diluted with saline.

### Platelet Count, Coagulation and Bleeding Time

All blood samples were collected into a plastic syringe containing a final concentration of 0.32% trisodium citrate. The platelet count was determined using a Technicon H₂ blood cell analyzer (Bayer Diagnostics, Tarrytown, N.Y.).

Prothrombin time (PT) and activated partial thromboplastin time (aPTT) were measured at 37° C. using a coagulometer (Clotex II, Hyland).

The template bleeding time was measured at the surface of the forearm using the SIMPLATE® II device (Organon Teknika, Durham, N.C.). The volar surface of the forearm was shaved, and a pressure cuff was applied and inflated to 40 mmHg. Time elapsed until the visual cessation of blood onto the filter paper was recorded as the bleeding time. Bleeding times were followed for up to ten minutes.

### Plasma Concentration of 82D6A3

Microtiter plates (96-well, Greiner, Frickenhausen, Germany) were coated overnight at 4° C. with 5 μg/ml (in PBS, 100 μl/well) goat anti-mouse IgG whole molecule (Sigma, St. Louis, Mo.). Plates were blocked with 3% milk powder (PBS, 250 μl/well) for two hours at room temperature (RT). Frozen plasma samples were thawed and incubated for five minutes at 37° C. before addition to the plate. Dilution series of the samples (1/2 in PBS) were made and incubated for two hours at RT. Goat anti-mouse IgG labeled with horse radish peroxidase (HRP) were added and were incubated for one hour at RT. Visualization was obtained with ortho-phenylenediamine (OPD, Sigma) and the coloring reaction was stopped with 4 mol/l H₂SO₄. The absorbance was determined at 490 nm. After each incubation step, plates were washed with PBS, 0.1% TWEEN® 20, three times after coating and blocking steps and twelve times elsewhere. The plasma concentration of mAb 82D6A3 in each sample was calculated from a standard curve. This curve was obtained by adding known amounts of mAb 82D6A3 to baboon plasma (free of antibody) and plating 1/2 dilutions in PBS (starting from 6 μg/ml).

vWF-Ag Levels

Determination of the vWF-Ag levels was performed essentially as described (K. Vanhoorelbeke et al., Thromb. Haemost. 2000, 83(1):107-113). Briefly, microtiter plates were coated with a polyclonal anti-vWF-Ig-solution (Dako, Glostrup, 20 Denmark). Plates were blocked with 3% milk powder and samples were added to the wells at 1/40 to 1/2560 dilutions (samples were diluted in PBS, 0.3% milk powder). Bound vWF was detected with rabbit anti-human vWF HRP antibodies (Dako). Visualization and wash steps were performed as described above. vWF-Ag levels were calculated from a standard curve obtained by adding 1/40 to 1/2560 dilutions to the coated wells of a human plasma pool, known to contain 10 μg/ml human vWF.

### vWF Occupancy

Microtiter plates (96-well) were coated overnight at 4° C. with 125 μl/well of a polyclonal anti-vWF-Ig-solution (Dako) (1/1000 in PBS). Plates were blocked with 3% milk powder solution (in PBS, 250 μl/well) for two hours at room temperature (RT). Plasma samples were incubated for five minutes at 37° C. before addition to the plate. Pure samples were added and dilution series (1/2 in PBS) were made. Samples were incubated for two hours at RT. Samples containing 100%-occupied vWF were obtained by adding a saturating amount of mAb 82D6A3 (6 μg/ml) to the corresponding baboon plasma. Bound mAb 82D6A3 was detected by addition of goat anti-mouse IgG-HRP (one hour at RT). Visualization and wash steps were performed as described above. The vWF-occupancy of each sample was calculated as follows: (A490 nm sample/A490 nm sample saturated with mAb 82D6A3)*100.

Determination of the vWF-Collagen Binding Activity

The ELISA was performed essentially as described (K. Vanhoorelbeke et al., Thromb. Haemost. 2000, 83(1):107-113). Briefly, microtiter plates were coated with human collagen type I (Sigma). Plates were blocked with 3% milk powder solution (in PBS, 250 μl/well). Pure sample and 1/2 dilution series were added. Bound vWF was detected with rabbit anti-human vWF-HRP antibodies. Binding of baboon vWF to collagen in the different blood samples was compared to the binding of vWF in the blood sample taken at time zero (pre-sample) which was set as 100%.

### Determination of vWF Binding to Collagen and Inhibition by F(ab) Fragment of 82D6A3

A 96-well plate was coated overnight with human collagen type I or III or calfskin collagen type 1 (25 μg/ml) and blocked. 2.5 μg/ml of recombinant vWF was used in the binding experiments. For the competition experiments, purified human vWF (0.5 μg/ml fc) or plasma (1/50 fc) was pre-incubated with a dilution series of 82D6A3 or its F(ab) fragment during 30 minutes in a pre-blocked 96-well plate. Then the mixtures were added to the blocked collagen-coated plate. After 90 minutes incubation, bound vWF was detected with a polyclonal anti-vWF-HRP conjugated antibody (Dako, Glostrup, Denmark) and visualization was performed with orthophenylenediamine (OPD, Sigma). The reaction was stopped with 4 mol/L H₂SO₄ and absorbance was determined at 490-630 nm. In between each incubation step the plates were washed three to nine times with PBS (0.1% TWEEN® 20).

### Flow Experiments

Plastic thermanox coverslips were rinsed with 40% ethanol and washed with water before spraying with human fibrillar collagen type I (1 mg/ml). Blood was taken from healthy volunteers who had not taken aspirin or analogues for the last ten days. The blood was anti-coagulated with 25 U/ml low molecular weight heparin (LMWH) (Leo Pharmaceuticals, Vilvoorde, Belgium). The perfusion experiments were performed in a Sakariassen-type flow chamber at 37° C., at wall shear rates of 600 s⁻¹, 1300 s⁻¹ and 2600 s⁻¹. The perfusion chamber and tubings were rinsed with plasma during 20 minutes and washed with 25 ml Hepes-buffered saline (HBS) before starting the experiment. In each experiment, 15 ml blood, pre-incubated for 15 minutes with an inhibitor as indicated, was perfused for five minutes. After the perfusion, coverslips were rinsed with 25 ml Hepes-buffered saline and put in 0.5% glutardialdehyde (ten minutes). Next, the coverslips were placed in methanol (five minutes), stained with May-Grunwald (three to five minutes) and Giemsa (15 to 20 minutes) and washed two times with distilled water. Coverslips were dried and analyzed with an image analyzer as described (J. Harsfalvi et al., Blood 1995, 85:705-711).

### Isolation of MoAb Binding Phages

Selection of phages was performed as follows. Biotinylated (see below) MoAb (10 μg) was bound to blocked streptavidin-coated magnetic beads (Dynal, Oslo, Norway). 2×10¹² phages (PBS, 0.2% milk powder) were first incubated with blocked streptavidin-coated beads for one hour to eliminate the streptavidin-binders. Next, the phages were added to the MoAb-containing beads and after 90 minutes, the input phages were removed and the beads were washed ten times with PBS (0.1% TWEEN® 20) to remove the non-specific binders. The bound phages were eluted with 0.1 mol/L glycine, pH 2.2, and the eluate was immediately neutralized with 1 mol/L Tris, pH 8. After amplification of the phages, additional rounds of panning were performed. Phages were amplified by infection of Escherichia coli TG1 cells and partially purified from the supernatant by polyethylene glycol precipitation. Individual phage-bearing E. coli were grown in a 96-well plate, and the supernatant was tested for the presence of 82D6A3-binding phages. Phage DNA was prepared and sequencing reactions were performed according to the T7-polymerase sequencing kit (Pharmacia) using the primer 5′-TGAATTTTCTGTATGAGG-3′ (SEQ ID NO: 11).

### Measurement of Phage Binding to 82D6A3

A 96-well plate was coated overnight with purified 82D6A3 (10 μg/mL). After two hours blocking with 2% milk powder, a dilution series of the individual phage clones in PBS with 0.2% milk powder was added to the wells and phages were incubated at room temperature for 90 minutes. Bound phages were detected after one hour incubation with a polyclonal anti-M13-HRP conjugated antibody (Pharmacia) and visualization was performed with OPD.

### Specificity of Phage Binding to 82D6A3

A 96-well plate was coated overnight with purified 82D6A3 (10 μg/ml). After two hours blocking with 2% milk powder, a dilution series of vWF or recombinant A3 domain was added. After a 30-minute pre-incubation, a constant amount of phages was added to the vWF/A3 containing wells. Ninety minutes later, bound phages were detected as described above. Competition between different phage clones for binding to 82D6A3 was analyzed as above, except that 2×10¹⁰/ml biotinylated phages of clone 1 were mixed with various concentrations of phages from clone 2, after which bound biotinylated phages were detected with streptavidin-HRP and OPD. MoAb and phages were biotinylated using NHS-LC-Biotin (Pierce, Rockford, Ill.) according to the manufacturer's instructions.

### Immunoblotting of Phages

Purified phage clones (2×10¹⁰/ml) were electrophoresed on a 10% SDS-PAGE gel under reducing and non-reducing conditions and electroblotted to a nitrocellulose membrane. After blocking the membrane with 4% skimmed milk in PBS, the membrane was incubated with 82D6A3 (2 μg/ml) during 90 minutes, followed by a one-hour incubation with GaM-HRP and developed using the ECL detection system from Amersham (Buckinghamshire, England). After each incubation step, the membrane was washed with PBS containing 0.05% TWEEN® 80.

### Example 15

### Results on the vWF Inhibition

### Anti-Thrombotic Effect

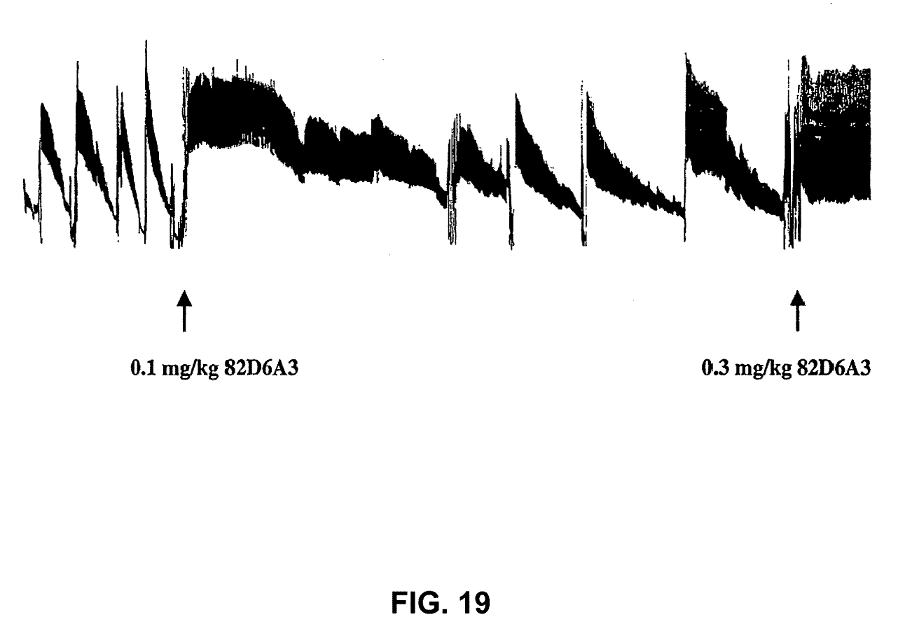

The frequency of the CFRs was not changed by injection of saline (107±7%). A dose of 100 μg/kg mAb 82D6A3 resulted in a significant reduction of the CFRs by 58.3+4.8% (FIG. 19). From a dose of 300 μg/kg upwards, the CFRs were completely abolished and could not be restored by increasing intimal damage or increasing stenosis (FIG. 20).

### Platelet Count, Coagulation and Bleeding Time

The platelet count was not significantly affected by injection of the different doses of mAb 82D6A3 (Table III). No significant changes of PT or aPTT were observed in any of the animals (data not shown). The bleeding time was slightly prolonged after injection of 300 μg/kg and 600 μg/kg mAb 82D6A3, but returned to baseline levels five hours later (Table III).

Ex Vivo mAb 82D6A3 Plasma Concentration, vWF-Ag Levels, vWF-Occupancy and vWF Collagen Binding

Plasma samples, taken after several time points (see Material and Methods) in each study, were analyzed for mAb 82D6A3 plasma levels, vWF-Ag levels, vWF-occupancy and collagen binding activity ex vivo.

Thirty minutes after injection of the different doses of mAb 82D6A3, a small decrease in vWF-Ag levels was observed, whereas an increase in vWF-Ag levels above baseline was consistently measured after 24 hours (Tables IV & V).

Measurement of the mAb 82D6A3 plasma levels revealed no decrease in mAb 82D6A3 plasma levels in the first three hours of the experiment. Then 69%, 23%, 7.6% mAb 82D6A3 was present after 300 minutes, 24 hours and 48 hours, respectively, when 300 μg/kg mAb 82D6A3 was administered (Table IV).

Injection of 100 μg/kg mAb 82D6A3 resulted in an ex vivo inhibition of the vWF-collagen binding of 31% (blood sample taken after one hour) (Table IV). At doses of 300 μg/kg and 600 μg/kg, no interaction between baboon vWF and collagen was observed in samples taken up to five hours after the administration of the mAb. Blood samples taken 24 hours after the injection of the drug revealed a recovery of the vWF-collagen interaction (Table IV).

At 300 minutes after administration, vWF-occupancy was 80% for the 100 μg/kg dose and near 100% for the 300 μg/kg and 600 μg/kg doses. vWF remained occupied for a long time: even 48 hours after the injection of mAb 82D6A3, still 63% of the vWF was occupied with mAb 82D6A3 (Table IV).

Relation Between the Ex Vivo vWF-Occupancy and Collagen Binding, the vWF-Occupancy and 82D6A3 Plasma Levels and Between vWF-Ag and 82D6A3 Plasma Levels

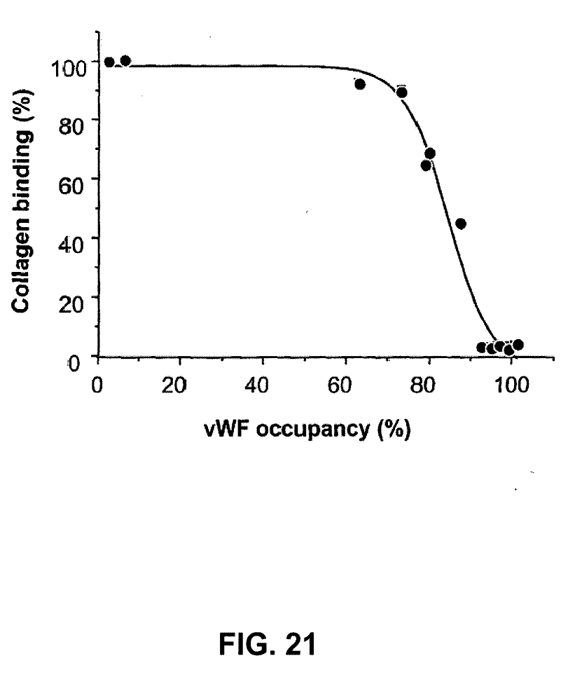

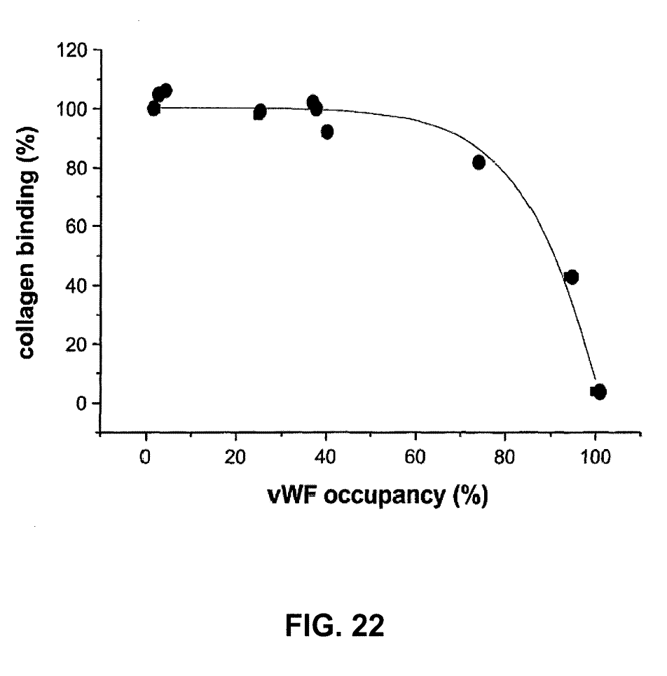

vWF-occupancy inversely correlated with vWF-binding to collagen: to obtain inhibition of vWF-binding to collagen, a vWF occupancy of at least 70% was required, with complete inhibition at 90 to 100% occupancy (FIG. 21). These data were confirmed by in vitro experiments, where different concentrations of mAb 82D6A3 were added to baboon plasma (FIG. 22): occupancy levels of up to 60% resulted in little inhibition of the vWF binding to collagen, while inhibition was observed when 70% to 100% of the vWF-binding sites for the antibody were occupied.

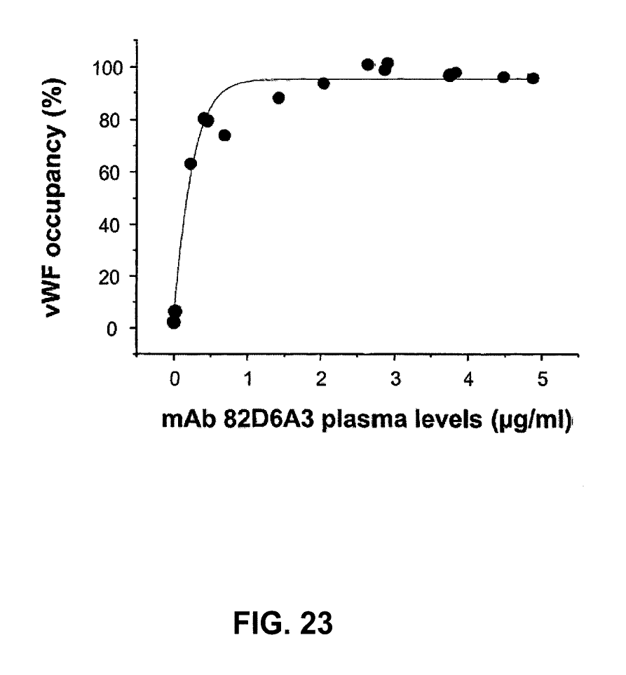

A good relation between 82D6A3 plasma levels and vWF-occupancy was also obtained with a maximum vWF-occupancy from about 1 μg/ml 82D6A3 onwards (FIG. 23).

### Characterization of 82D6A3 and its F(ab)-Fragment Both Under Static and Flow Conditions

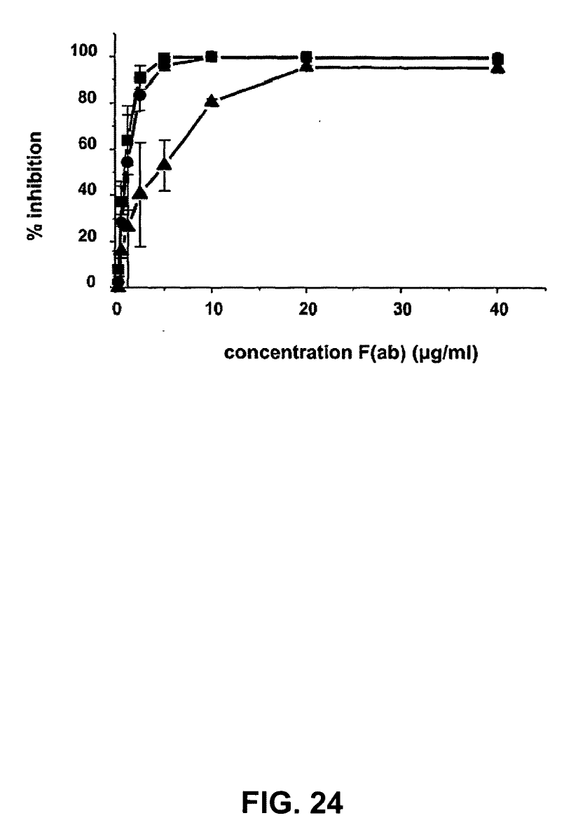

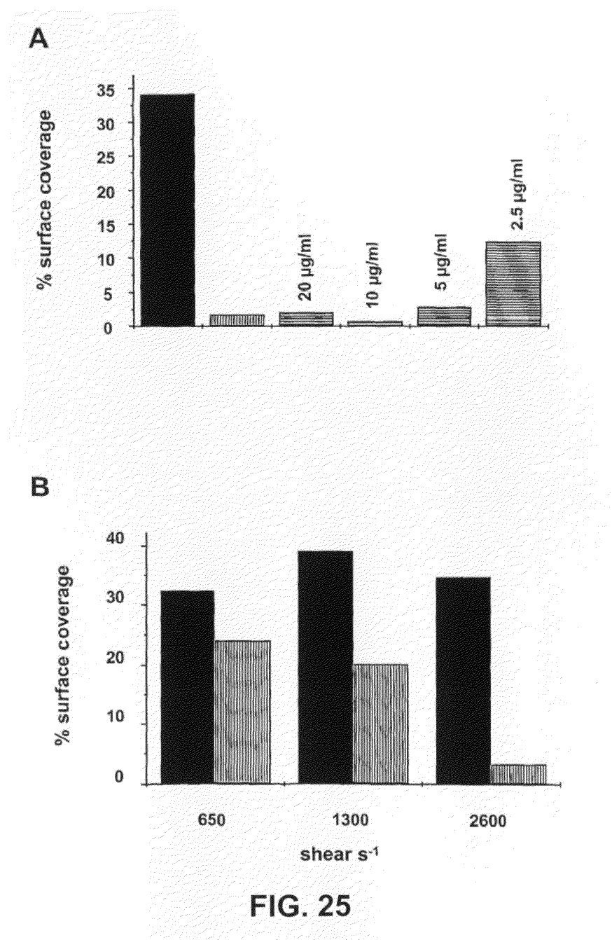

82D6A3 is an anti-vWF antibody that binds with high affinity to vWF (Kd: 0.4 nM), to the SpI proteolytic fragment and the recombinant vWF-A3 domain. Both the MoAb and its F(ab) fragment are able to inhibit plasma or purified vWF-binding to human collagen type I in a specific and dose-dependent manner with an IC50 of 20 ng/ml for the MoAb and 1 μg/ml for the F(ab) fragment (FIG. 24). The vWF binding to human collagen type III and calfskin collagen type I was inhibited in the same way. Next, 82D6A3 and its F(ab) fragment were tested under flow conditions at different shear rates (600, 1300 and 2600 s⁻¹). At a shear rate of 1300 s⁻¹, both the intact MoAb and F(ab) completely inhibited platelet deposition at 1 to 5 μg/ml and 10 μg/ml, respectively (FIG. 25, Panel A) and the inhibitory effect increased with the shear applied (FIG. 25, Panel B).

### Epitope Mapping of 82D6A3 by Means of Phage Display

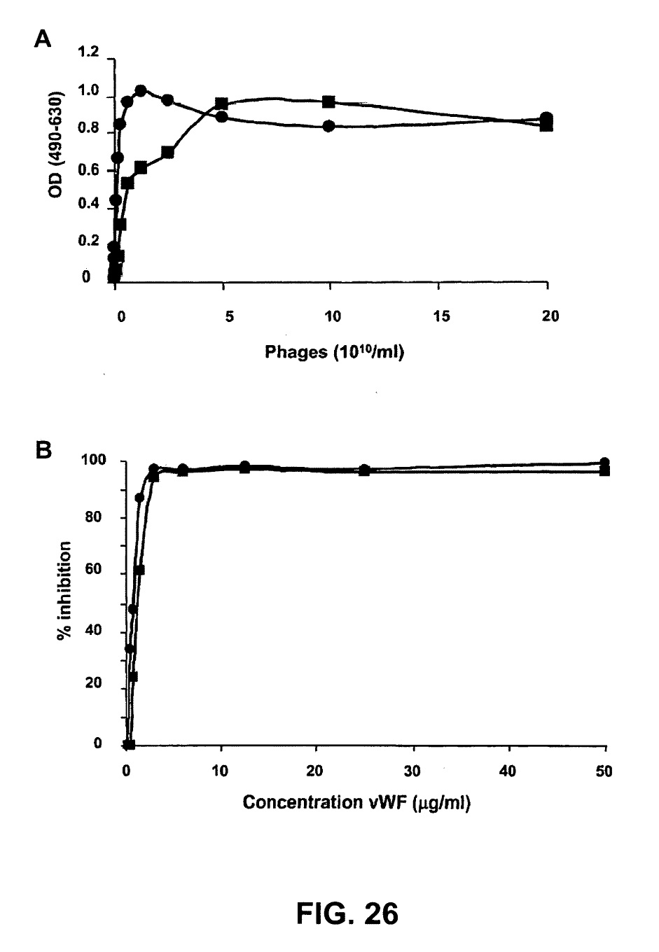

Two peptide phage display libraries, a linear pentadecamer and a cyclic hexamer, were used. After three rounds of biopanning with the pentadecamer library, individual clones were grown and tested for their ability to bind to 82D6A3 (FIG. 26, Panel A). To determine whether the phages were binding to the antigen-binding pocket of the antibody, binding phage-clones were subjected to a competition ELISA to test whether vWF and the A3 domain were able to compete with the phages for binding to the 82D6A3 (FIG. 26, Panel B). From the different inhibitory clones that were thus identified, the sequence was determined, which resulted in the identification of two sequences: GDCFFGFLNSPWRVC (SEQ ID NO:12) (L15G8) and RSSYWVYSPWRFISR (SEQ ID NO:13) (L15C5). Both sequences shared the same four amino acid sequence SPWR (SEQ ID NO: 14).

However, the affinity of the L15G8 phage for binding to the MoAb was higher than that of the L15C5 phage.

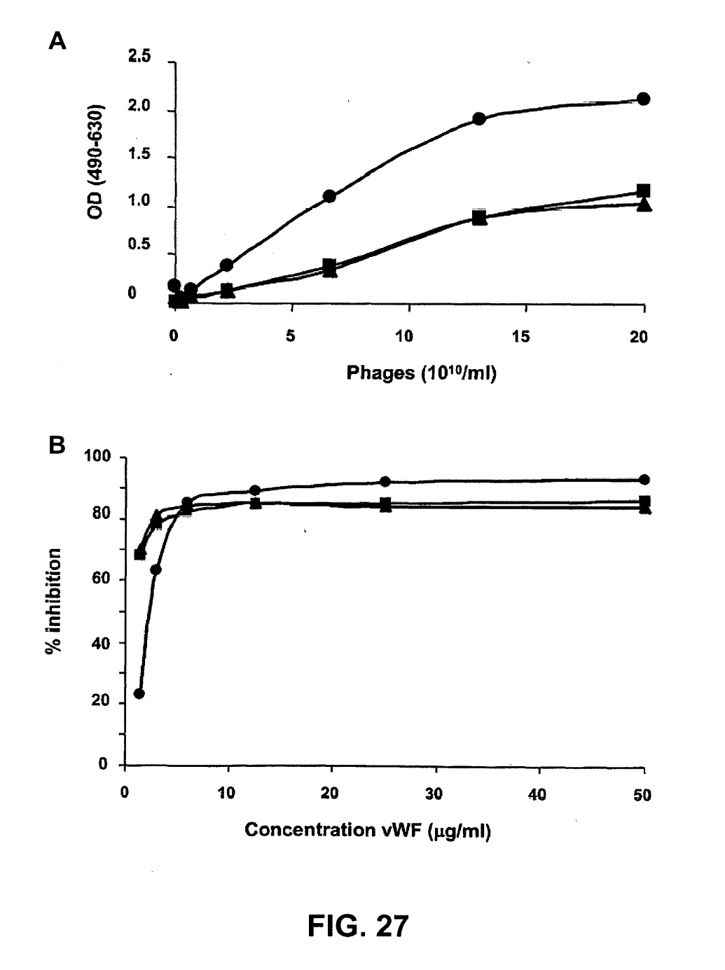

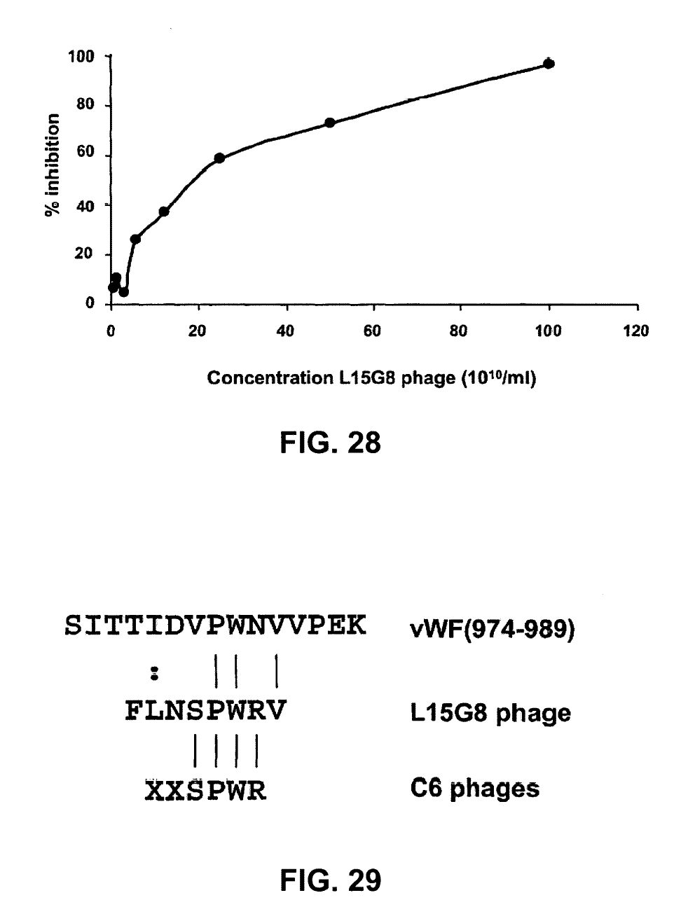

After four rounds of biopanning with the cyclic hexamer library, individual clones were checked for binding to 82D6A3 (FIG. 27, Panel A) and for inhibition by vWF and the A3 domain (FIG. 27, Panel B). From the phage-clones that did compete, ssDNA was prepared and the sequence determined. Eight out of thirteen clones displayed CMTSPWRC (SEQ ID NO:15) (C₆H₅), four out of thirteen CRTSPWRC (SEQ ID NO:16) (C6G12), and one had the CYRSPWRC (SEQ ID NO:17) (C6A12) sequence. These sequences can be aligned with the L15 sequences that also contained the SPWR (SEQ ID NO:14) sequence. The L15G8 and C₆H₅ phage did compete with each other for binding to 82D6A3 (FIG. 28), which let us conclude that the epitope SPWR (SEQ ID NO:14) may be part of the epitope of 82D6A3. Furthermore, by immunoblotting of the L15G8 and C₆H₅ phages, it was demonstrated that the two cysteines present in both clones are forming a disulfide bridge, necessary for recognition by 82D6A3 (FIG. 29). Both the L15G8 sequence and the C₆H₅ sequence could be tentatively aligned in the vWF sequence more especially within the A3 domain.

TABLE III

| | Dose | | | | | |
|---|---|---|---|---|---|---|
| | 100 μg/kg (n = 3) | | 300 μg/kg (n = 3) | | 600 μg/kg (n = 2) | |
| | Platelet count | Bleeding time | Platelet count | Bleeding time | Platelet count | Bleeding time |
| min | (10³/μl) | (min) | (10³/μl) | (min) | (10³/μl) | (min) |
| 0 | 286 ± 54 | 2.7 ± 0.4 | 286 ± 54 | 2.7 ± 0.4 | 335 | 1.8 |
| 30 | 292 ± 65 | 2.7 ± 0.4 | 265 ± 41 | 4.6 ± 0.6 | 320 | 3.5 |
| 60 | 289 ± 49 | 3.5 ± 2.1 | 287 ± 53 | 7.3 ± 2.5 | 313 | 5.5 |
| 150 | / | / | 309 ± 83 | 6.4 ± 3.1 | 356 | 5 |
| 300 | / | / | 282 ± 7 | 3.15 ± 1.2 | 334 | 3 |
| 24 h | / | / | 312 ± 46 | 3.25 ± 0.3 | 347 | / |
| 48 h | / | / | 306 ± 79 | 3 | / | / |

TABLE IV

| | vWF-Ag levels | | MoAb 82D6A3 levels | | vWF occupancy | | collagen binding | |
|---|---|---|---|---|---|---|---|---|
| | (μg/ml) | | (μg/ml) | | (%) | | (%) | |
| min | 100 μg/kg | 300 μg/kg | 100 μg/kg | 300 μg/kg | 100 μg/kg | 300 μg/kg | 100 μg/kg | 300 μg/kg |
| 0 | 10.2 ± 1.7 | 10.2 ± 1.7 | 0 | 0 | 2.3 ± 1.3 | 2.3 ± 1.3 | 101 ± 7 | 101 ± 7 |
| 30 | 10.2 ± 2.5 | 8.8 ± 1.4 | 0.4 ± 0.07 | 2.9 ± 0.3 | 80 ± 10.8 | 102 ± 10.4 | 64 ± 7 | 4 ± 1 |
| 60 | 8.9 ± 1.4 | 9.1 ± 2.4 | 0.4 ± 0.1 | 2.8 ± 0.3 | 80 ± 2.4 | 99 ± 10.6 | 69 ± 9 | 4 ± 1 |
| 150 | | 9.7 ± 2.7 | | 2.6 ± 0.1 | | 101 ± 7.6 | | 4 ± 1 |
| 300 | | 8.8 ± 0.1 | | 2.0 ± 0.5 | | 94 ± 0.9 | | 4 ± 1 |
| 24 h | | 12.8 ± 1.3 | | 0.7 ± 0.2 | | 74 ± 31 | | 91 ± 18 |
| 48 h | | 13.2 ± 0.8 | | 0.2 ± 0.01 | | 63 ± 7.8 | | 93 ± 0 |

TABLE V

| | VWF-Ag | mAb 82D6A3 | vWF | collagen |
|---|---|---|---|---|
| | levels | levels | occupancy | binding |
| | (μg/ml) | (μg/ml) | (%) | (%) |
| 0 min | 14 ± 1.7 | 0 | 6.9 ± 0.1 | 100 ± 0 |
| 30 min | 11.5 ± 0.9 | 4.5 ± 0.5 | 96 ± 1 | 4 ± 0.2 |
| 60 min | 10.8 ± 0.1 | 4.8 ± 0.7 | 96 ± 0.2 | 3.5 ± 0.2 |
| 150 min | 11.9 ± 1.8 | 3.8 ± 0.5 | 97 ± 4 | 3.52 ± 0.2 |
| 300 min | 10.5 ± 0 | 3.8 ± 0.6 | 97 | 4 |
| 24 h | 22.9 ± 0 | 1.4 ± 0.01 | 88 | 45 |

1. A method of treating or preventing an occlusive syndrome in the cerebral vascular system or a transient cerebral infarct leading to thrombotic stroke, to ischemic stroke, or to acute stroke or a cerebral infarct of the thrombotic stroke, ischemic stroke or acute stroke type in a subject, the method comprising:
administering to a subject in need of such treatment a therapeutically effective amount of an antibody or antibody fragment that inhibits platelet adhesion, wherein said antibody or antibody fragment is a binder of a platelet receptor or of a platelet receptor activator.

2. The method according to claim 1, wherein activation of GPIb-mediated pathways is inhibited by monovalent antibody or antibody fragment.

3. The method according to claim 2, wherein the binding of the platelet glycoprotein (GP) Ib receptor to von Willebrand factor on the endothelial surface of the cerebral vascular system is inhibited by the monovalent antibody or antibody fragment.

4. The method according to claim 2, wherein the monovalent antibody or antibody fragment is a ligand of platelet glycoprotein (GP) Ib or platelet glycoprotein (GP) Ibα.

5. The method according to claim 4, wherein the monovalent antibody or antibody fragment is administered at a dose between 50 and 800 mg per subject.

6. The method according to claim 4, wherein the monovalent antibody or antibody fragment is administered at a dose between 150 and 500 mg per subject.

7. The method according to claim 4, wherein the monovalent antibody or antibody fragment is administered before occurrence of occlusive syndrome in the cerebral vascular system.

8. The method according to claim 4, wherein the monovalent antibody or antibody fragment is administered after the occurrence of the occlusive syndrome in the cerebral vascular system.

9. The method according to claim 2, wherein the antibody or antibody fragment is a ligand of von Willebrand factor.

10. The method according to claim 9, wherein the antibody or antibody fragment is administered at a dose between 50 and 800 mg per subject.

11. The method according to claim 9, wherein the antibody or antibody fragment is administered at a dose between 150 and 500 mg per subject.

12. The method according to claim 9, wherein the antibody or antibody fragment is administered before occurrence of occlusive syndrome in the cerebral vascular system.

13. The method according to claim 9, wherein the antibody or antibody fragment is administered after occurrence of occlusive syndrome in the cerebral vascular system.

14. The method according to claim 1, wherein the binding of platelet glycoprotein (GP) VI receptor to collagen is inhibited by the monovalent antibody or antibody fragment.

15. The method according to claim 14, wherein the monovalent antibody or antibody fragment is a ligand to platelet glycoprotein (GP) VI receptor.

16. The method according to claim 15, wherein the monovalent antibody or antibody fragment is administered at a dose between 50 and 800 mg per subject.

17. The method according to claim 15, wherein the monovalent antibody or antibody fragment is administered at a dose between 150 and 500 mg per subject.

18. The method according to claim 15, wherein the monovalent antibody or antibody fragment is administered before occurrence of occlusive syndrome in the cerebral vascular system.

19. The method according to claim 15, wherein the monovalent antibody or antibody fragment is administered after occurrence of occlusive syndrome in the cerebral vascular system.

20. A method of treating or preventing occlusive syndrome in the cerebral vascular system of the thrombotic stroke, ischemic stroke or acute stroke type or a transient cerebral infarct leading to thrombotic stroke, ischemic stroke or acute stroke in a subject, the method comprising:
administering to a subject in need of such treatment a therapeutically effective amount of a monovalent antibody or antibody fragment that inhibits activation of platelet glycoprotein (GP) Ib receptor or that inhibits activation of the platelet glycoprotein (GP) VI receptor.

21. The method according to claim 20, wherein the binding of von Willebrand factor to the platelet glycoprotein (GP) Ib receptor is inhibited by the monovalent antibody or antibody fragment.

22. The method according to claim 20, wherein the binding of the platelet glycoprotein (GP) Ib receptor to von Willebrand factor on the endothelial surface of the cerebral vascular system is inhibited by the monovalent antibody or antibody fragment.

23. The method according to claim 21, wherein the monovalent antibody or antibody fragment is a ligand of platelet glycoprotein (GP) lb.

24. The method according to claim 21, wherein the monovalent antibody or antibody fragment is administered at a dose between 50 and 800 mg per subject.

25. The method according to claim 21, wherein the monovalent antibody or antibody fragment is administered at a dose between 150 and 500 mg per subject.

26. The method according to claim 21, wherein the monovalent antibody or antibody fragment is administered before occurrence of occlusive syndrome in the cerebral vascular system.

27. The method according to claim 21, wherein the monovalent antibody or antibody fragment is administered after occurrence of occlusive syndrome in the cerebral vascular system.

28. The method according to claim 21, wherein the monovalent antibody or antibody fragment is a ligand of von Willebrand factor.

29. The method according to claim 28, wherein the monovalent antibody or antibody fragment is administered at a dose between 50 and 800 mg per subject.

30. The method according to claim 28, wherein the monovalent antibody or antibody fragment is administered at a dose between 150 and 500 mg per subject.

31. The method according to claim 28, wherein the monovalent antibody or antibody fragment is administered before occurrence of occlusive syndrome in the cerebral vascular system.

32. The method according to claim 28, wherein the monovalent antibody or antibody fragment is administered after occurrence of occlusive syndrome in the cerebral vascular system.

33. The method according to claim 20, wherein the binding of platelet glycoprotein (GP) VI receptor to collagen is inhibited by the monovalent antibody or antibody fragment.

34. The method according to claim 33, wherein the monovalent antibody or antibody fragment is a ligand to platelet glycoprotein (GP) VI receptor.

35. The method according to claim 34, wherein the monovalent antibody or antibody fragment is administered at a dose between 50 and 800 mg per subject.

36. The method according to claim 34, wherein the monovalent antibody or antibody fragment is administered at a dose between 150 and 500 mg per subject.

37. The method according to claim 34, wherein the monovalent antibody or antibody fragment is administered before the occurrence of the occlusive syndrome in the cerebral vascular system.

38. The method according to claim 34, wherein the monovalent antibody or antibody fragment is administered after the occurrence of the occlusive syndrome in the cerebral vascular system.

39. The method according to claim 2, wherein said monovalent antibody or antibody fragment is administrated in the acute phase of cerebral infarct or cerebral ischemia.

40. The method according to claim 20, wherein said monovalent antibody or antibody fragment is administrated in the acute phase of cerebral infarct or cerebral ischemia.

41.-44. (canceled)

45. The method according to claim 1, comprising further administering to the subject a therapeutically effective amount of vascular endothelial growth factor, a fragment, a derivative or a homologue thereof.

46. The method according to claim 1, comprising further administering to the subject a therapeutically effective amount of a placenta growth factor (PIGF), a fragment, a derivative or a homologue thereof or a vascular endothelial growth factor (VEGF), a fragment, a derivative or a homologue thereof or a combination of PIGF and VEGF or a VEGF/PIGF heterodimer.

47. The method according to claim 1, comprising further administering to the subject a therapeutically effective amount of a α2-AP neutralizing antibody or derivatives thereof, preferably monovalent antibodies such as monoclonal Fab fragment or a ScFv, comprising both a heavy chain variable domain and/or a light chain variable domain of antibody fragments comprising a variable domain, such as a heavy chain variable domain and/or a light chain variable domain or single domain antibodies or single domain antibody fragments comprising only the variable domain, such as a heavy chain variable domain and/or a light chain variable domain or a therapeutically effective amount of a compounds which neutralize α2-AP or increase fibrinolysis of the group consisting of plasmin, mini-plasmin (lacking the first four kringles), micro-plasmin (lacking all five kringles), or human plasmin-forming proteins, including lys-plasminogen or similar substances.

48. The method according to claim 20, comprising further administering to the subject a therapeutically effective amount of vascular endothelial growth factor, a fragment, a derivative or a homologue thereof.

49. The method according to claim 20, comprising further administering to the subject a therapeutically effective amount of a placenta growth factor (PIGF), a fragment, a derivative or a homologue thereof, or a vascular endothelial growth factor (VEGF), a fragment, a derivative or a homologue thereof, or a combination of PIGF and VEGF or a VEGF/PIGF heterodimer.

50. The method according to claim 20, comprising further administering to the subject a therapeutically effective amount of an α2-AP neutralizing antibody or derivatives thereof, preferably monovalent antibodies such as monoclonal Fab fragment or a ScFv comprising both a heavy chain variable domain and/or a light chain variable domain of antibody fragments comprising a variable domain, such as a heavy chain variable domain and/or a light chain variable domain or single domain antibodies or single domain antibody fragments comprising only the variable domain, such as a heavy chain variable domain and/or a light chain variable domain or a therapeutically effective amount of a compounds which neutralize α2-AP or increase fibrinolysis of the group consisting of plasmin, mini-plasmin (lacking the first four kringles), microplasmin (lacking all five kringles), or human plasmin-forming proteins, including lys-plasminogen or similar substances.


SEQUENCE LISTING (17 sequences)

SEQ ID NO: 1 - DNA, 359 bp, Mus musculus
gacattgagctcacccagtctccagcaatcatgtctgcatctccaggggagaaggtcaccatgacctgcagtgccagttcaagtgttaattacatgcactggttccagcaggagtcgggcaccttccccaaaagaaggatttatgacacatccaaactggcttctggagtccctgctcgcctcagtggcagtgggtctgggacagaattcaccctggaaatcagtagagtgaaggctgaggatgtgggtgtgtattactgtcaacaacttgtagagtatccgctcacgttcggtgctgggaccaagctggagctgaaacgggctgatgctgcaccaactgtatccatcttcaagcttcc

SEQ ID NO: 2 - DNA, 363 bp, Mus musculus
caggtgcagctgcaggagtctggacctggcctggtggcgccctcacagagcctgtccatcacttgcactgtctctgggatttcattaaacagatatggtgtacactgggttcgccagcctccaggaaagggtctggagtggctgggagtaatatggactggtggaagcacaaattataattcggctctcatgtccagactgagcatcagcaaagacaactccaagagccaagttttcttaaaaatgaacagtctgcagactgatgacacagccatgtactactgtgccagagatcgatctactatgattacggcctatgctatggactactggggccaagggaccacggtcaccgtctcctca

SEQ ID NO: 3 - protein, 119 aa, Mus musculus
DIELTQSPAIMSASPGEKVTMTCSASSSVNYMHWFQQESGTFPKRRIYDTSKLASGVPARLSGSGSGTEFTLEISRVKAEDVGVYYCQQLVEYPLTFGAGTKLELKRADAAPTVSIFKL

SEQ ID NO: 4 - protein, 121 aa, Mus musculus
QVQLQESGPGLVAPSQSLSITCTVSGISLNRYGVHWVRQPPGKGLEWLGVIWTGGSTNYNSALMSRLSISKDNSKSQVFLKMNSLQTDDTAMYYCARDRSTMITAYAMDYWGQGTTVTVSS

SEQ ID NO: 5 - DNA, 23 bp, Mus musculus
  misc_feature (6)..(6): n is either C or T
caggtnnanctgcagnagtcngg

SEQ ID NO: 6 - DNA, 34 bp, Mus musculus
tgaggagacggtgaccgtggtcccttggccccag

SEQ ID NO: 7 - DNA, 24 bp, Mus musculus
gacattgagctcacccagtctcca

SEQ ID NO: 8 - DNA, 32 bp, Mus musculus
ggaagcttgaagatggatacagttggtgcagc

SEQ ID NO: 9 - DNA, 18 bp, Mus musculus
ttcctcgacgctaacctg

SEQ ID NO: 10 - DNA, 18 bp, Mus musculus
gatttaatctgtatcagg

SEQ ID NO: 11 - DNA, 18 bp, Artificial Sequence
  Chemically synthesized - primer
tgaattttctgtatgagg

SEQ ID NO: 12 - protein, 15 aa, Artificial Sequence
  Chemically synthesized
GDCFFGFLNSPWRVC

SEQ ID NO: 13 - protein, 15 aa, Artificial Sequence
  Chemically synthesized
RSSYWVYSPWRFISR

SEQ ID NO: 14 - protein, 4 aa, Artificial Sequence
  Chemically synthesized
SPWR

SEQ ID NO: 15 - protein, 8 aa, Artificial Sequence
  Chemically synthesized
CMTSPWRC

SEQ ID NO: 16 - protein, 8 aa, Artificial Sequence
  Chemically synthesized
CRTSPWRC

SEQ ID NO: 17 - protein, 8 aa, Artificial Sequence
  Chemically synthesized
CYRSPWRC